(19) United States
(12) Patent Application Publication
(10) Pub. No.: US 2022/0257616 A1
(43) Pub. Date: Aug. 18, 2022

(54) COMPOSITIONS FOR MAINTAINING OR MODULATING MIXTURES OF ETHER LIPID MOLECULES IN A TISSUE OF A HUMAN SUBJECT

(71) Applicant: BAKER HEART AND DIABETES INSTITUTE, Melbourne (AU)

(72) Inventor: Peter John Meikle, Melbourne (AU)

(21) Appl. No.: 17/597,659

(22) PCT Filed: Jul. 17, 2020
(86) PCT No.: PCT/AU2020/050742

§ 371 Date: Jan. 17, 2022

(30) Foreign Application Priority Data
Jul. 17, 2019 (AU) 2019902527

(51) Int. Cl. A61K 31/661; A61P 3/02; A61P 3/04; A61P 3/10; A61K 31/231; A23L 33/00; A23L 33/12

(52) U.S. Cl. CPC A61K 31/661; A61P 3/02; A61P 3/04; A23V 2002/00; A61K 31/231; A23L 33/40; A23L 33/12; A61P 3/10

(57) ABSTRACT

Provided herein is a composition comprising a mixture of ether lipid molecules of Formula (I) as defined herein. The composition is for invivo maintenance of ether lipids at levels and/or ratios associated with a non-disease state, or wherein the composition is for in vivo modification of ether lipids towards levels and/or ratios associated with a non-disease state. Also provided herein are methods of assessing subject for metabolic disease or dyslipidemia, comprising measuring relative abundance of one or more ether lipid side chains in a biological sample from a subject, methods of preventing or treating disorders such as metabolic disease or dyslipidemia, or methods of preventing conditions such as obesity and asthma, particularly in infants, involving administering a composition as defined herein.

### FIELD OF DISCLOSURE

This disclosure relates generally to compositions and methods for maintaining or modulating mixtures of ether lipid molecules in a tissue of a human subject.

### BACKGROUND ART

The reference in this specification to any prior publication, or to any matter which is known, is not, and should not be taken as an acknowledgment or admission or any form of suggestion that that prior publication or known matter forms part of the common general knowledge in the field of endeavour to which this specification relates.

Bibliographic details of documents referred to are listed at the end of the specification.

Metabolic disease, encompassing obesity, insulin resistance and type 2 diabetes, and dyslipidemia (thought to be associated with abnormal (usually elevated) amounts of unhealthy lipids such as triglycerides and cholesterol) are a major drain on health systems. Early intervention has the potential to substantially improve health and reduce health expenditure. However, such intervention should ideally be inexpensive and low risk to apply to a large subset of the population. Modulation of the lipid dysregulation by statins represents a proven and attractive option for early intervention; and is arguably one of the most significant developments in terms of health outcomes in the past century. However, statins have only reduced negative cardiovascular outcomes by ˜30%, which leaves the majority of the disease burden uncontrolled. Further to this, the dramatic increase in obesity and diabetes (themselves risk factors for cardiovascular disease) has offset much of the risk reduction provided by statins and so new prevention/treatment measures are required.

Lipids are among the least studied molecules of the metabolome. Plasmanyl- and/or plasmenyl-phospholipids are a unique class of ether phospholipids that are major components of cell membranes. Their biophysical role in cell membranes has been studied while knowledge concerning their biological roles is an important area of new research. Plasmalogens are primarily present as alkenylphosphatidylcholine (PC) and alkenylphosphatidylethanolamine (PE) species. They are characterised by a cis vinyl ether bond linking an alkyl chain to the sn-1 position of the glycerol backbone. They also have an acyl linked fatty acid in the sn-2 position. Plasmalogens are often esterified with polyunsaturated fatty acids such as arachidonic acid (20:4) and the omega-3 fatty acid docosahexaenoic acid (22:6, a major constituent of fish oil), whereas the vinyl ether linked residue is usually saturated (i.e. no double bonds present in the chain other than the vinyl ether group) or monounsaturated (i.e. one double bond present in the chain in addition to the vinyl ether group).

Plasmalogen biosynthesis is a complex process involving multiple enzymes within the peroxisome and endoplasmic reticulum. The rate-limiting step in this pathway is the formation of the long chain fatty alcohol by fatty acyl-CoA reductase 1 and 2 (Far-1/2). It is possible to bypass the rate-limiting step in plasmalogen synthesis through the oral administration of naturally occurring alkylglycerols (1-O-alkylglycerol or 1-O-alkyl-2,3-diacylglycerol). These can be incorporated directly into the phospholipid pathway, and so bypass the peroxisome. This leads to an increase in circulating and tissue plasmalogens. Although alkylglycerols are present in our diet, the levels in typical diets are insufficient to significantly boost our plasmalogen levels. Shark liver oil is rich in alkylglycerols and is currently used as a dietary supplement to reduce inflammation and improve immune function. Alkylglycerols can also be synthesised, providing a future avenue for an environmentally sustainable source of these compounds (Magnusson C D., et al Tetrahedron. 2011; 67:1821-36; Shi Y,. et al, Green Chemistry. 2010; 12(12)).

There is determined herein a need for better ether lipid supplementation programmes.

### SUMMARY OF THE DISCLOSURE

There is provided a composition comprising a mixture of ether lipid molecules for in vivo maintenance of ether lipids at levels and/or ratios associated with a non-disease state, or wherein the composition is for in vivo modification of ether lipids towards levels and/or ratios associated with a non-disease state. In one embodiment, the composition may usefully form nutritional supplements, food or cosmetic products. In one embodiment, the compositions may be used in therapeutic, prophylactic and maintenance administrations.

Accordingly, in one aspect, the present application provides a composition comprising a mixture of ether lipid molecules of Formula (I):

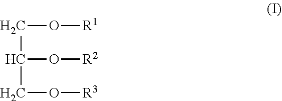

wherein
R¹ is an alkyl or alkenyl group;
R² is hydrogen or

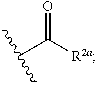

and
R³ is hydrogen,

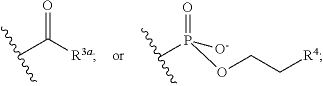

wherein
R^{2a} and R^{3a} are each an alkyl or alkenyl group;
R⁴ is —N(Me)₃⁺ or —NH₃⁺; and

wherein the composition is for in vivo maintenance of ether lipids at levels and/or ratios associated with a non-disease state, or wherein the composition is for in vivo modification of ether lipids towards levels and/or ratios associated with a non-disease state.

In some embodiments, the composition is for in vivo maintenance or in vivo modification of plasmanyl- and/or plasmenyl-phospholipid levels and/or ratios.

In some embodiments, the composition comprises ether lipid molecules having an 18:0 alkyl R¹ group, and ether lipid molecules having an 18:1 alkenyl R¹ group. In some embodiments, the composition is for in vivo maintenance of ether lipids at or in vivo modification of ether lipids towards an in vivo plasmalogen ether lipid profile in which the ether lipids have a molar ratio of 18:0 ether groups to 18:1 ether groups in the range of from 1.2:1 to 2.5:1. In some embodiments, the composition is for in vivo maintenance of ether lipids at or in vivo modification of ether lipids towards an in vivo plasmalogen ether lipid profile in which the ether lipids have a molar percent of 18:0 ether groups in the range of from 32.6% to 45.8%, and a molar percent of 18:1 ether groups in the range of from 18.6% to 27.9%. In some embodiments, the composition comprises ether lipids having a molar ratio of 18:0 alkyl R¹ groups to 18:1 alkenyl R¹ groups in the range of from 1.2:1 to 2.5:1.

In some embodiments, the composition comprises ether lipid molecules having an 18:1 alkenyl R¹ group, and ether lipid molecules having a 16:0 alkyl R¹ group. In some embodiments, the composition is for in vivo maintenance of ether lipids at or in vivo modification of ether lipids towards an in vivo plasmalogen ether lipid profile in which the ether lipids have a molar ratio of 18:1 ether groups to 16:0 ether groups in the range of from 0.5:1 to 1:1. In some embodiments, the composition is for in vivo maintenance of ether lipids at or in vivo modification of ether lipids towards an in vivo plasmalogen ether lipid profile in which the ether lipids have a molar percent of 18:1 ether groups in the range of from 18.6% to 27.9%, and a molar percent of 16:0 ether groups in the range of from 26.8% to 37.4%. In some embodiments, the composition comprises ether lipids having a molar ratio of 18:1 alkenyl R¹ groups to 16:0 alkyl R¹ groups in the range of from 0.5:1 to 1:1.

In some embodiments, the composition comprises ether lipid molecules having an 18:0 alkyl R¹ group, and ether lipid molecules having a 16:0 alkyl R¹ group. In some embodiments, the composition is for in vivo maintenance of ether lipids at or in vivo modification of ether lipids towards an in vivo plasmalogen ether lipid profile in which the ether lipids have a molar ratio of 18:0 ether groups to 16:0 ether groups in the range of from 0.9:1 to 1.7:1. In some embodiments, the composition is for in vivo maintenance of ether lipids at or in vivo modification of ether lipids towards an in vivo plasmalogen ether lipid profile in which the ether lipids have a molar percent of 18:0 ether groups in the range of from 32.6% to 45.8%, and a molar percent of 16:0 ether groups in the range of from 26.8% to 37.4%. In some embodiments, the composition comprises ether lipids having a molar ratio of 18:0 alkyl R¹ groups to 16:0 alkyl R¹ groups in the range of from 0.9:1 to 1.7:1.

In some embodiments, the composition comprises ether lipid molecules having an 18:1 alkenyl R¹ group, ether lipid molecules having an 18:0 alkyl R¹ group, and ether lipid molecules having a 16:0 alkyl R¹ group. In some embodiments, the composition is for in vivo maintenance of ether lipids at or in vivo modification of ether lipids towards an in vivo plasmalogen ether lipid profile in which the ether lipids have a molar ratio of 18:1 ether groups to 18:0 ether groups to 16:0 ether groups of about 1:1.7:1.4. In some embodiments, the composition is for in vivo maintenance of ether lipids at or in vivo modification of ether lipids towards an in vivo plasmalogen ether lipid profile in which the ether lipids have a molar percent of 18:1 ether groups in the range of from 18.6% to 27.9%, a molar percent of 18:0 ether groups in the range of from 32.6% to 45.8%, and a molar percent of 16:0 ether groups in the range of from 26.8% to 37.4%. In some embodiments, the composition comprises ether lipids having a molar ratio of 18:1 alkenyl R¹ groups to 18:0 alkyl R¹ groups to 16:0 alkyl R¹ groups of about 1:1.7:1.4. In some embodiments, wherein ether lipids having an 18:1 alkenyl R¹ group, ether lipids having an 18:0 alkyl R¹ group, and ether lipids having an 16:0 alkyl R¹ group together comprise at least 50% of the ether lipids in the composition.

In some embodiments, the composition additionally comprises ether lipids having R¹groups selected from the group consisting of 15:0 alkyl, 17:0 alkyl, 19:0 alkyl, 20:0 alkyl, and 20:1 alkenyl.

In some embodiments, the composition comprises ether lipids wherein R² and R³ is hydrogen.

In some embodiments, the composition comprises ether lipids in which R² is hydrogen and R³ is

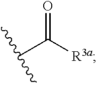

and

R^{3a} is selected from the group consisting of a saturated alkyl hydrocarbon, a monounsaturated alkenyl hydrocarbon and a polyunsaturated alkenyl hydrocarbon.

In some embodiments, the composition comprises ether lipids in which R³ is hydrogen and R² is

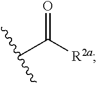

and

R^{2a} is selected from the group consisting of a saturated alkyl hydrocarbon, a monounsaturated alkenyl hydrocarbon and a polyunsaturated alkenyl hydrocarbon.

In some embodiments, the composition comprises ether lipids in which

R² is:

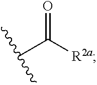

R³ is

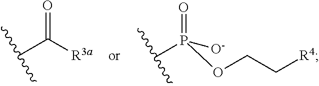

wherein

R^{2a} is selected from the group consisting of a saturated alkyl hydrocarbon, a monounsaturated alkenyl hydrocarbon and a polyunsaturated alkenyl hydrocarbon;

R^{3a} is selected from the group consisting of a saturated alkyl hydrocarbon, a monounsaturated alkenyl hydrocarbon and a polyunsaturated alkenyl hydrocarbon; and

R⁴ is —N(Me)₃⁺ or NH₃⁺.

In some embodiments, the composition comprises ether lipid molecules having a 20:4 acyl alkenyl R² and/or R³ group, ether lipids having a 22:6 acyl alkenyl R² and/or R³ group, and ether lipids having an 18:2 acyl alkenyl R² and/or R³ group. In some embodiments, the composition is for in vivo maintenance of ether lipids at or in vivo modification of ether lipids towards an in vivo plasmalogen ether lipid profile in which the ether lipids have a molar ratio of 20:4 acyl groups to 22:6 acyl groups to 18:2 acyl groups of about 3:1.2:1. In some embodiments, the composition is for in vivo maintenance of ether lipids at or in vivo modification of ether lipids towards an in vivo plasmalogen ether lipid profile in which the ether lipids have acyl groups in which the molar percent of 20:4 acyl groups is in the range of from 31.3% to 52.5%, the molar percent of 22:6 acyl groups is in the range of from 9.3% to 23.9%, and the molar percent of 18:2 acyl groups is in the range of from 7.6% to 19.9%. In some embodiments, the composition comprises ether lipids having a molar ratio of 20:4 acyl alkenyl groups to 22:6 acyl alkenyl groups to 18:2 acyl alkenyl groups of about 3:1.2:1.

In some embodiments, the composition comprises free fatty acids. In some embodiments, the composition comprises omega fatty acids, such as omega-3 or omega-6 fatty acids. In some embodiments, the composition is an ether lipid-containing composition according to the Examples.

In some embodiments, the composition is in the form of a composition for addition to a food or beverage.

In some embodiments, the composition is in the form of a product which is a dietary supplement, capsule, syrup, liquid, food or beverage.

In another aspect there is provided a composition comprising a mixture of ether lipid molecules of Formula (I):

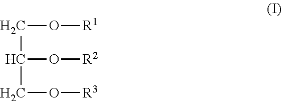

wherein

R¹ is an alkyl or alkenyl group;

R² is hydrogen or

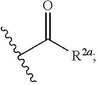

and

R³ is hydrogen,

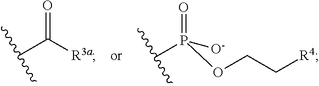

wherein

R^{2a} and R^{3a} are each an alkyl or alkenyl group; and

R⁴ is —N(Me)₃⁺ or —NH₃⁺, and

wherein the composition is present in the form of a product which is a liquid infant formula milk, an infant formula milk powder, a supplement for addition to infant formula milk, a supplement for addition to infant food, or an infant dietary supplement.

In some embodiments, the composition comprises ether lipid molecules having an 18:0 R¹ group, and ether lipid molecules having an 18:1 R¹ group.

In some embodiments, the composition is for in vivo maintenance of ether lipids at or in vivo modification of ether lipids towards an in vivo plasmalogen ether lipid profile in which the ether lipids have a molar ratio of 18:0 ether groups to 18:1 ether groups of from 0.74:1 to 1.60:1. In some embodiments, the composition is for in vivo maintenance of ether lipids at or in vivo modification of ether lipids towards an in vivo plasmalogen ether lipid profile in which the ether lipids have a molar ratio of 18:0 ether groups to 18:1 ether groups of from 0.95:1 to 1.25:1.

In some embodiments, the composition is for in vivo maintenance of ether lipids at or in vivo modification of ether lipids towards an in vivo plasmalogen ether lipid profile in which the ether lipids have a molar percent of 18:0 ether groups in the range of from 27.7% to 39.6%, and a molar percent of 18:1 alkenyl ether groups in the range of from 24.7% to 37.4%. In some embodiments, the composition is for in vivo maintenance of ether lipids at or in vivo modification of ether lipids towards an in vivo plasmalogen ether lipid profile in which the ether lipids have a molar percent of 18:0 ether groups in the range of from 31.8% to 35.8%, and a molar percent of 18:1 alkenyl ether groups in the range of from 28.6% to 32.5%.

In some embodiments, the composition comprises ether lipids having a molar ratio of 18:0 R¹ groups to 18:1 R¹ groups in the range of from 0.30:1 to 1.20:1. In some embodiments, the composition comprises ether lipids having a molar ratio of 18:0 le groups to 18:1 R¹ groups in the range of from 0.35:1 to 0.70:1.

In some embodiments, the composition comprises ether lipid molecules having an 18:1 R¹ group, and ether lipid molecules having a 16:0 R¹ group.

In some embodiments, the composition is for in vivo maintenance of ether lipids at or in vivo modification of ether lipids towards an in vivo plasmalogen ether lipid profile in which the ether lipids have a molar ratio of 18:1 ether groups to 16:0 ether groups in the range of from 0.79:1 to 2.9:1. In some embodiments, the composition is for in vivo maintenance of ether lipids at or in vivo modification of ether lipids towards an in vivo plasmalogen ether lipid profile in which the ether lipids have a molar ratio of 18:1 ether groups to 16:0 ether groups in the range of from 1:1 to 1:1.35.

In some embodiments, the composition is for in vivo maintenance of ether lipids at or in vivo modification of ether lipids towards an in vivo plasmalogen ether lipid profile in which the ether lipids have a molar percent of 18:1 ether groups in the range of from 24.7% to 37.4%, and a molar percent of 16:0 ether groups in the range of from 30.1% to 41.7%.In some embodiments, the composition is for in vivo maintenance of ether lipids at or in vivo modification of ether lipids towards an in vivo plasmalogen ether lipid profile in which the ether lipids have a molar percent of 18:1 ether groups in the range of from 28.6% to 32.5%, and a molar percent of 16:0 ether groups in the range of from 33.5% to 37.4%.

In some embodiments, the composition comprises ether lipids having a molar ratio of 18:1 R¹ groups to 16:0 R¹ groups in the range of from 1:0.55 to 1:2.3. In some embodiments, the composition comprises ether lipids having a molar ratio of 18:1 R¹ groups to 16:0 R¹ groups in the range of from 1:1.05 to 1:1.55.

In some embodiments, the composition comprises ether lipid molecules having an 18:0 R¹ group, and ether lipid molecules having a 16:0 R¹ group.

In some embodiments, the composition is for in vivo maintenance of ether lipids at or in vivo modification of ether lipids towards an in vivo plasmalogen ether lipid profile in which the ether lipids have a molar ratio of 18:0 ether groups to 16:0 ether groups in the range of from 0.66:1 to 1.3:1. In some embodiments, the composition is for in vivo maintenance of ether lipids at or in vivo modification of ether lipids towards an in vivo plasmalogen ether lipid profile in which the ether lipids have a molar ratio of 18:0 ether groups to 16:0 ether groups in the range of from 0.85:1 to 1.1:1.

In some embodiments, the composition is for in vivo maintenance of ether lipids at or in vivo modification of ether lipids towards an in vivo plasmalogen ether lipid profile in which the ether lipids have a molar percent of 18:0 ether groups in the range of from 27.7% to 39.6%, and a molar percent of 16:0 ether groups in the range of from 30.1% to 41.7%. In some embodiments, the composition is for in vivo maintenance of ether lipids at or in vivo modification of ether lipids towards an in vivo plasmalogen ether lipid profile in which the ether lipids have a molar percent of 18:0 ether groups in the range of from 31.8% to 35.8%, and a molar percent of 16:0 ether groups in the range of from 33.5% to 37.4%.

In some embodiments, the composition comprises ether lipids having a molar ratio of 18:0 R¹ groups to 16:0 R¹ groups in the range of from 0.44:1 to 1.82:1. In some embodiments, the composition comprises ether lipids having a molar ratio of 18:0 R¹ groups to 16:0 R¹ groups in the range of from 1:1.05 to 1:2.

In some embodiments, the composition comprises ether lipid molecules having an 18:1 R¹ group, ether lipid molecules having a 18:0 R¹ group, and ether lipid molecules having a 16:0 R¹ group.

In some embodiments, the composition is for in vivo maintenance of ether lipids at or in vivo modification of ether lipids towards an in vivo plasmalogen ether lipid profile in which the ether lipids have a molar ratio of 18:1 ether groups to 18:0 ether groups to 16:0 ether groups of about 0.9:1.0:1.05.

In some embodiments, the composition is for in vivo maintenance of ether lipids at or in vivo modification of ether lipids towards an in vivo plasmalogen ether lipid profile in which the ether lipids have a molar percent of 18:1 ether groups in the range of from 24.7% to 37.45%, a molar percent of 18:0 ether groups in the range of from 27.7% to 39.6%, and a molar percent of 16:0 ether groups in the range of from 30.1% to 41.7%. In some embodiments, the composition is for in vivo maintenance of ether lipids at or in vivo modification of ether lipids towards an in vivo plasmalogen ether lipid profile in which the ether lipids have a molar percent of 18:1 ether groups in the range of from 28.6% to 32.5%, a molar percent of 18:0 ether groups in the range of from 31.8% to 35.8%, and a molar percent of 16:0 ether groups in the range of from 33.5% to 37.4%.

In some embodiments, the composition comprises ether lipids having a molar ratio of 18:0 R¹ groups to 16:0 R¹ groups to 18:1 R¹ groups in the range of from 0.5:1:3 to 2:1:1.

In some embodiments, ether lipids having an 18:1 R¹ group, ether lipids having an 18:0 R¹ group, and ether lipids having a 16:0 R¹ group together comprise at least 50% of the ether lipids in the composition.

In some embodiments, the composition additionally comprises ether lipids having le groups selected from the group consisting of 16:0, 18:2, 20:0 and 20:1.

In some embodiments, the composition comprises ether lipids wherein R² and R³ is hydrogen.

In some embodiments, the composition comprises ether lipids in which R² is hydrogen and R³ is

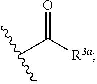

and

R^{3a} is selected from the group consisting of a saturated alkyl hydrocarbon, a monounsaturated alkenyl hydrocarbon and a polyunsaturated alkenyl hydrocarbon.

In some embodiments, the composition comprises ether lipids in which R³ is hydrogen and R² is

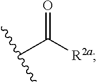

and

R^{2a} is selected from the group consisting of a saturated alkyl hydrocarbon, a monounsaturated alkenyl hydrocarbon and a polyunsaturated alkenyl hydrocarbon.

In some embodiments, the composition comprises ether lipids in which

R² is:

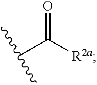

R³ is

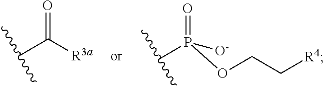

wherein

R^{2a} is selected from the group consisting of a saturated alkyl hydrocarbon, a monounsaturated alkenyl hydrocarbon and a polyunsaturated alkenyl hydrocarbon;

R^{3a} is selected from the group consisting of a saturated alkyl hydrocarbon, a monounsaturated alkenyl hydrocarbon and a polyunsaturated alkenyl hydrocarbon; and

R⁴ is —N(Me)3⁺ or —NH₃⁺.

In some embodiments, the composition according to any one of claims 34 to 56, wherein the composition comprises free fatty acids.

In some embodiments, the composition comprises omega-3 or omega-6 fatty acids.

In some embodiments, the composition comprises ether lipid molecules of Formula (I) in an amount such that, when present in liquid infant formula milk, the concentration of total ether lipid molecules of Formula (I) is in the range of from 75 to 140 μM.

In some embodiments, the composition is prepared by mixing a plurality of ether lipids, in ratios and/or levels corresponding with ratios and/or levels associated with a non-disease state in vivo. In some embodiments, the composition is prepared by mixing a plurality of ether lipids, in ratios and/or levels corresponding with ratios and/or levels associated with modifying in vivo ether lipid molecules towards ratios and/or levels corresponding to a non-disease state in vivo. The present application provides methods for determining these rations and or levels.

In one embodiment, pharmaceutical or physiological compositions are contemplated comprising the composition as described herein together with a carrier which in one embodiment is a pharmaceutically acceptable carrier.

There is also provided a method of maintaining ether lipids in a subject at levels and/or ratios associated with a non-disease state, or of modifying ether lipids in a subject towards levels and/or ratios associated with a non-disease state, comprising administering an effective amount of a composition as defined herein to the subject.

In some embodiments, the method is for maintenance or modification of plasmanyl- and/or plasmenyl-phospholipid levels and/or ratios in a subject.

In another aspect, the present application provides a method of assessing a subject for or with a metabolic disease, diabetes, cardiovascular disease, obesity, overweight, fatty liver disease, an inflammatory condition or dyslipidemia in a tissue or a risk of developing same, the method comprising measuring the relative abundance of one or more ether lipid molecules in a biological sample from a subject to obtain a subject ether lipid molecule profile, and (ii) determining the similarity or difference between the ether lipid molecule profile obtained in (i) and a reference ether lipid molecule profile.

In some embodiments, the reference ether lipid molecule profile is the profile characteristic of a healthy/non-disease individual and comprises:

ether lipids having a molar ratio of 18:1 alkenyl ether to 18:0 alkyl ether to 16:0 alkyl ether groups of about 1:1.7:1.4;

and/or

ether lipids having a molar percent of 18:1 alkenyl ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%.

In another aspect, the present application provides a method of treating or preventing metabolic disease, diabetes, cardiovascular disease, obesity, overweight, fatty liver disease, an inflammatory condition or dyslipidemia in a subject, the method comprising (i) determining the relative abundance of one or more ether lipid molecules in a biological sample from a subject to obtain a subject ether lipid molecule profile, and (ii) administering a composition as defined herein contingent upon the similarity or difference between the ether lipid molecule profile obtained in (i) and a reference ether lipid molecule profile.

In some embodiments, the reference ether lipid molecule profile comprises the profile characteristic of a healthy/non-disease individual. In one embodiment the profile comprises:

ether lipids having a molar ratio of 18:1 alkenyl ether to 18:0 alkyl ether to 16:0 alkyl ether groups of about 1:1.7:1.4;

and/or

ether lipids having a molar percent of 18:1 alkenyl ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%. In another embodiment the level or ratio of 20:0 alkyl ether is determined and compared. In another embodiment the level or ratio of 20:4 acyl ether is determined and compared. In another embodiment the level or ratio of 18:1 acyl ether is determined and compared. In another embodiment the level or ratio of 20:3 acyl ether is determined and compared. In another embodiment the level or ratio of one or more of 15:0, 17:0, 18:0, 19:0 alkenyl ether is/are determined and compared.

In another aspect, the present application provides a method of treating or preventing metabolic disease, diabetes, cardiovascular disease, obesity, overweight, fatty liver disease, an inflammatory condition or dyslipidemia in a subject, or promoting in a subject ether lipids at levels and/or ratios associated with a non-disease state, the method comprising administering an effective amount of a composition as defined herein to the subject.

There is also provided a method of preventing asthma, an inflammatory condition, obesity or overweight in an infant subject, the method comprising administering an effective amount of a composition as defined herein to the infant subject.

There is also provided a composition as defined herein for use in therapy.

There is also provided a composition as defined herein for use in treating or preventing metabolic disease, diabetes, cardiovascular disease, obesity, overweight, fatty liver disease, an inflammatory condition or dyslipidemia in a subject.

There is also provided a composition as defined herein for use in preventing asthma, an inflammatory condition, obesity or overweight in an infant subject.

There is also provided use of a composition as defined herein for the manufacture of a medicament for the treatment or prevention of metabolic disease, diabetes, cardiovascular disease, obesity, overweight, fatty liver disease, an inflammatory condition or dyslipidemia in a subject.

There is also provided use of a composition as defined herein for the manufacture of a medicament for the prevention of asthma, obesity or overweight in an infant subject. In one embodiment, the present application provides a method of vivo maintenance in a subject of ether lipids at levels and/or ratios associated with a non-disease state, or modification in a subject of ether lipids towards levels and/or ratios associated with a non-disease state, the method comprising administering to the subject an effective amount of a composition comprising ether lipid molecules having an 18:0 alkyl R1 group, and ether lipid molecules having an 18:1 alkenyl R1 group. In some embodiments, the composition comprises a mixture of ether lipid molecules having a molar ratio of 18:0 alkyl ether groups to 18:1 alkenyl ether groups in the range of from 1.2:1 to 2.5:1. In some embodiments, the ether lipids have a molar percent of 18:0 alkyl ether groups in the range of from 32.6% to 45.8%, and a molar percent of 18:1 alkenyl ether groups in the range of from 18.6% to 27.9%. In one embodiment, the mixture comprises ether lipid molecules having an 18:1 alkenyl R1 group, and ether lipid molecules having a 16:0 alkyl R1 group. In some embodiments, the composition comprises ether lipids having a molar ratio of 18:1 alkenyl R1 groups to 16:0 alkyl R1 groups in the range of from 0.5:1 to 1:1. In some embodiments, the ether lipids have a molar percent of 18:1 alkenyl ether groups in the range of from 18.6% to 27.9%, and a molar percent of 16:0 alkyl ether groups in the range of from 26.8% to 37.4%. In one embodiment, the mixture comprises ether lipid molecules having an 18:0 alkyl R1 group, and ether lipid molecules having a 16:0 alkyl R1 group.

In some embodiments, the composition comprises ether lipids having a molar ratio of 18:0 alkyl R1 groups to 16:0 alkyl R1 groups in the range of from 0.9:1 to 1.7:1. In some embodiments, the ether lipids have a molar percent of 18:0 alkyl ether groups in the range of from 32.6% to 45.8%, and a molar percent of 16:0 alkyl ether groups in the range of from 26.8% to 37.4%. In some embodiments, the composition comprises ether lipid molecules having an 18:1 alkenyl R1 group, ether lipid molecules having an 18:0 alkyl R1 group, and ether lipid molecules having a 16:0 alkyl R1 group. In some embodiments, the composition comprises ether lipids having a molar ratio of 18:1 alkenyl R1 groups to 18:0 alkyl R1 groups to 16:0 alkyl R1 groups of about 1:1.7:1.4. In some embodiments, the ether lipids have a molar percent of 18:1 alkenyl R1 ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%. In some embodiments, wherein ether lipids having an 18:1 alkenyl R1 group, ether lipids having an 18:0 alkyl R1 group, and ether lipids having an 16:0 alkyl R1 group together comprise at least 50% of the ether lipids in the composition. In some embodiments, the composition additionally comprises ether lipids having R1 groups selected from the group consisting of 15:0 alkyl, 17:0 alkyl, 19:0 alkyl, 20:0 alkyl, and 20:1 alkenyl.

In some embodiments, the composition administered comprises ether lipids wherein R² and R³ is hydrogen.

In some embodiments, the composition comprises ether lipids in which R² is hydrogen and R³ is

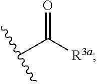

and

R^{3a} is selected from the group consisting of a saturated alkyl hydrocarbon, a monounsaturated alkenyl hydrocarbon and a polyunsaturated alkenyl hydrocarbon.

In some embodiments, the composition comprises ether lipids in which R³ is hydrogen and R² is

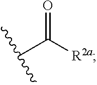

and

R^{2a} is selected from the group consisting of a saturated alkyl hydrocarbon, a monounsaturated alkenyl hydrocarbon and a polyunsaturated alkenyl hydrocarbon.

In some embodiments, the composition comprises ether lipids in which

R²is:

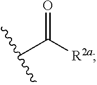

R³ is

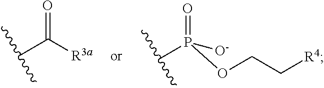

wherein

R^{2a} is selected from the group consisting of a saturated alkyl hydrocarbon, a monounsaturated alkenyl hydrocarbon and a polyunsaturated alkenyl hydrocarbon;

R^{3a} is selected from the group consisting of a saturated alkyl hydrocarbon, a monounsaturated alkenyl hydrocarbon and a polyunsaturated alkenyl hydrocarbon; and

R⁴ is —N(Me)₃⁺ or NH₃⁺.

In some embodiments, the composition comprises ether lipid molecules having a 20:4 acyl alkenyl R2 and/or R3 group, ether lipids having a 22:6 acyl alkenyl R2 and/or R3 group, and ether lipids having an 18:2 acyl alkenyl R2 and/or R3 group. In some embodiments, the composition is for in vivo maintenance of ether lipids at or in vivo modification of ether lipids towards an in vivo plasmalogen ether lipid profile in which the ether lipids have a molar ratio of 20:4 acyl alkenyl groups to 22:6 acyl alkenyl groups to 18:2 acyl alkenyl groups of about 3:1.2:1. In some embodiments, the composition is for in vivo maintenance of ether lipids at or in vivo modification of ether lipids towards an in vivo plasmalogen ether lipid profile in which the ether lipids have acyl alkenyl groups in which the molar percent of 20:4 acyl alkenyl groups is in the range of from 31.3% to 52.5%, the molar percent of 22:6 acyl alkenyl groups is in the range of from 9.3% to 23.9%, and the molar percent of 18:2 acyl alkenyl groups is in the range of from 7.6% to 19.9%. In some embodiments, the composition comprises ether lipids having a molar ratio of 20:4 acyl alkenyl groups to 22:6 acyl alkenyl groups to 18:2 acyl alkenyl groups of about 3:1.2:1. In some embodiments, the composition comprises free fatty acids. In some embodiments, the composition comprises omega fatty acids, such as omega-3 or omega-6 fatty acids. In some embodiments, the composition is an ether lipid-containing composition according to one or more of the Examples.

### BRIEF DESCRIPTION OF THE FIGURES

The patent or application file contains at least one drawing executed in colour. Copies of this patent or patent application publication with colour drawing(s) will be provided by the Patent Office upon request and payment of the necessary fee.

The accompanying drawings, which are incorporated into and form a part of the specification, illustrate several embodiments of the present disclosure and, together with the description, serve to explain the principles of the disclosure.

FIG. 1 shows the distributions of the relative abundances of PE(P) alkenyl and acyl chains. Violin plots illustrating the distributions of the relative abundances of either alkenyl chains (left) or acyl chains (right) amongst plasma PE(P) species, as measured across the 9,928 participants of the AusDiab cohort. The most abundant PE(P) alkenyl chains are O-18:0 (39.18%), O-16:0 (32.09%), and O-18:1 (23.25%), while the most abundant acyl chains are 20:4 (41.89%), 22:6 (16.55%), and 18:2 (13.77%).

FIG. 2 shows distributions of the relative abundances of PE(P) alkenyl and acyl chains in the healthy population. Violin plots illustrating the distributions of the relative abundances of either alkenyl chains (left) or acyl chains (right) amongst plasma PE(P) species, as measured in all AusDiab participants who did not have diabetes and were between the ages of 25 and 34, BMI (Body Mass Index) of 20-25, FBG<6.0, 2h-PLG<7.8, total cholesterol<5.17 mM and triglycerides<1.68 mM cohort.

FIG. 3 shows compositional distribution of the three most abundant alkenyl chains in PE(P) species. Ternary diagrams representing the alkenyl composition (for the 3 most abundant alkenyl chains) of plasma PE(P) species within Normoglycemic (healthy), Prediabetes and Diabetes groups in the AusDiab cohort. Groups are shown as different coloured markers (left) or as the 95% boundaries (right).

FIG. 4 shows compositional distribution of the three most abundant acyl chains in PE(P) species. Ternary diagram representing the acyl composition (for the 3 most abundant acyl chains) of plasma PE(P) species within Normoglycemic (healthy), Prediabetes and Diabetes groups in the AusDiab cohort. Groups are shown as different coloured markers (left) or as the 95% boundaries (right).

FIG. 5 shows association of PE(P) alkenyl chain composition with diabetes. Logistic regression was used to estimate the odds ratios (and confidence intervals thereof) of diabetes versus non-diabetes controls incurred by increasing the relative abundance of each of the alkenyl chains (adjusted for age, sex and BMI), using data from all participants of the AusDiab cohort (n=9,928).

FIG. 6 shows association of PE(P) acyl chain composition with diabetes. Logistic regression was used to estimate the odds ratios (and confidence intervals thereof) of diabetes versus non-diabetes controls incurred by increasing the relative abundance of each of the acyl chains (adjusted for age, sex and BMI), using data from all participants of the AusDiab cohort (n=9,928). Significant associations were found of 18:1 and 20:3 acyl chains with diabetes.

FIG. 7 shows association of PE(P) alkenyl chain composition with incident diabetes. Logistic regression was used to estimate the odds ratios (and confidence intervals thereof) of incident diabetes (218 participants) versus participants who did not develop diabetes (5,510 participants) incurred by increasing the relative abundance of each of the alkenyl chains (adjusting for age, sex and BMI), using data from the AusDiab cohort.

These results obtained in an incident diabetes setting confirmed those observed in the prevalent prediabetes/diabetes setting: of the plasma PE(P) alkenyl chains, the relative abundance of O-16:0 appears as a strong risk factor for metabolic disease.

FIG. 8 shows association of PE(P) acyl chain composition with incident diabetes. Logistic regression was used to estimate the odds ratios (and confidence intervals thereof) of incident diabetes (218 participants) versus participants who did not develop diabetes (5,510 participants) incurred by increasing the relative abundance of each of the acyl chains.

FIG. 9 shows the human shark liver oil supplementation study design. There were 5 visits in total for this study, which lasted for 9 weeks in total. There were 2 treatment periods separated by a 3-week washout period. In the first visit, the participant underwent a medical examination to assess if they are eligible for this study. Eligible participants were called for a 2nd visit in which they were randomized to take either alkyrol (shark liver oil gel caps) or placebo. The patients discontinued the treatment/placebo from the third to fourth visits (washout period) to allow time for lipid metabolism to normalise. At visit 4, the participants commenced the alternative treatment for 3 weeks. At visit 5 the participants underwent the same examinations as visit 1 to asses any change throughout the study period.

FIG. 10 shows 1-O-Alkyl-/1-O-alkenyl-glycerol composition in SLO. Pie chart representing the 1-O-alkyl- /1-O-alkenyl-glycerol composition of the SLO used in this supplementation study

FIG. 11 shows the effect of alkyl/alkenyl glycerol supplementation on plasma lipid classes. Bar plots showing the mean percentage change of the lipid class concentrations in the placebo and treatment groups. Whiskers represent standard error of the mean. The nominal significance of the treatment effect was determined using Repeated Measures ANOVA; * indicates P<0.05, ** indicates P<0.01 and *** indicates P<0.001.

FIG. 12 shows the effect of alkyl/alkenyl glycerol supplementation on plasma lipid classes. Bar plots showing the mean percentage change of the lipid class concentrations relative to total phosphatidylcholine concentration in the placebo and treatment groups. Whiskers represent standard error of the mean. The nominal significance of the treatment effect was determined using Repeated Measures ANOVA; * indicates P<0.05, ** indicates P<0.01 and *** indicates P<0.001.

FIG. 13 shows that SLO supplementation affects plasma PE(P) alkenyl chain composition. Ternary diagrams represent the top 3 alkenyl (left) and top 3 acyl (right) chain compositions in plasma PE(P) lipids before and after supplementation with either placebo or SLO (n=10 participants per group).

FIG. 14 shows relative alkenyl abundances amongst plasma PE(P) lipids in the SLO supplementation study. Bar plots representing the mean relative abundances (whiskers: +/−1 SD) of alkenyl chains within PE(P) lipids before/after placebo/SLO treatment irrespective of intervention order. Red stars (***: p<0.001) indicate nominal significance of the change induced by SLO treatment.

FIG. 15 shows alkenyl composition of mouse tissue PE(P) lipids. Ternary diagram representing PE(P) alkenyl composition of various organ samples in mice fed with a chow diet.

FIG. 16 shows total plasma PE(P) concentration across diets. Violin plots representing distributions of total plasma PE(P) levels for 6 diets (pmol/mL). AKG significantly increases total PE(P) levels (estimate=+4479 pmol/mL; p-value<0.05), and so does SLO (estimate=+3322 pmol/mL per 1% of SLO; p-value<0.05) (based on a linear model, adjusted R squared 0.292).

FIG. 17 shows alkenyl composition of plasma PE(P)s following different diets. Ternary diagram showing the effect of different diet types on plasma PE(P) alkenyl composition.

FIG. 18 shows alkenyl composition of plasma PE(P)s following SLO supplementation. Ternary diagram showing the effect of different SLO concentrations on plasma PE(P) alkenyl composition. The response is dose-dependent: higher SLO concentration leads to higher O-18:1 levels. It can be noticed that the mice given mid-range SLO concentration (0.75%) have a plasma composition (33% O-16:0; 35% O-18:0; 32% O-18:1) that is quite close to that of the chow diet (36% O-16:0; 34% O-18:0; 29% O-18:1; see FIG. 17), suggesting that this level of supplementation may counteract the compositional effect of the HFD.

FIG. 19 shows alkenyl composition of adipose tissue PE(P)s following SLO supplementation. Ternary diagram showing the effect of different SLO concentrations on adipose tissue PE(P) alkenyl composition. FIG. 18 shows that increasing levels of SLO supplementation increased the 18:1 part in plasma (roughly 25% to 40%), concomitantly reducing the O-16:0 and O-18:0 parts (35% to 30% and 40% to 30%). FIG. 19 shows that increasing levels of SLO supplementation have a different effect on adipose tissue: O-18:1 is still increased (12% to 23%), however the O-18:0 part is maintained (at about 25-26%) with only O-16:0 being decreased (63% to 51%).

FIG. 20 shows the biosynthetic pathway of plasmalogens. The formation of 1-O-alky-DHAP in the peroxisome is the rate-limiting step. Dietary alkyl/alkenyl glycerols can bypass the rate-limiting peroxisomal biosynthetic steps (red pathway). Metabolites are shown in red and black: DHAP: dihydroxyacetone phosphate, GPC: glycerophospho-choline, GPE: glycerophospho-ethanolamine. Enzymes are shown in blue circles: C-PT: choline phosphotransferase, DHAP-AT: DHAP acyltransferase, E-PT: ethanolamine phosphotransferase, Far1/2: fatty acyl-CoA reductase 1 or 2.

FIG. 21 shows Principal Component Analysis of maternal and infant plasma. Principal Component Analysis scores plot (across the first 2 principal components) for maternal (antenatal, 28-week gestation, purple), cord (“00m”, brown), and infant (06m/12m/48m, age of infant in months, red, yellow and blue respectively) plasma lipidomic data from the Barwon Infant Study.

FIG. 22 shows Principal Component Analysis of infant plasma at six months of age. PCA scores plot (first 2 PCs) for BIS infant plasma lipidomics samples at 6 months of age, coloured by breastfeeding status (currently breastfeeding, red; breastfed in the last 9 days, green; last breastfed more than 9 days ago or never: blue; unknown, purple).

FIG. 23 shows association of breast feeding status with lipid species. Forest plots showing the association of breastfeeding status (currently versus not currently breastfeeding) on the individual lipid species (grey dot: non-significant; pink dot: nominally significant; red dot: significant after multiple testing correction) and class totals (empty diamond: non-significant; light purple diamond: nominally significant; dark purple diamond: significant after multiple testing correction) measured in both 6-month (left) and 12-month (right) old infant plasma from the BIS, correcting for covariates such as child gender, gestational age, current age, and weight. Associations are reported as fold-change relative to non currently breastfed, with 95% confidence intervals indicated for species and classes reaching at least nominal significance.

FIG. 24 shows alkenyl and acyl chain composition of PE(P) in plasma from 6-month old infants. Bar plots showing the average percentage of PE-Ps containing each alkenyl (top) or acyl (bottom) chain, split by breastfeeding status (currently breastfed, dark grey; breastfed in the last 9 days, grey; breastfed 10 days ago or more, light grey), with whiskers showing the standard deviations, in plasma from BIS infants of 6 months of age.

FIG. 25 shows ternary plots of alkenyl and acyl chain composition of PE(P) in plasma from 6-month old infants. Ternary diagrams showing the PE-P alkenyl (top) and acyl (bottom) sidechain composition for the top 3 most abundant chains (resp. 16:0, 18:0, 18:1 and 18:2, 20:4, 22:6), coloured by (left) or split by breastfeeding status (right 3 diagrams) in plasma from BIS infants of 6 months of age.

FIG. 26 shows composition of major TG(O) species in plasma from 6-month old infants. Bar plot showing the average percentage of TG(O) species amongst total TG(O), split by breastfeeding status (currently breastfed, dark grey; breastfed in the last 9 days, grey; breastfed more than 10 days ago or more, light grey) in plasma from BIS infants of 6 months of age.

FIG. 27 shows principal component analysis of the lipid species in breast milk samples. Principal component analysis was performed on the lipidomic data from the BIS breast milk samples. PCA scores plot (first 2 PCs), showing breast milk samples from each sampling age (1 month, red; 6 months, green; 12 months, blue).

FIG. 28 shows PE(P) alkenyl and acyl chain composition in breast milk samples. Alkenyl and acyl chain composition of the PE-P species present in breast milk samples from the BIS were calculated. Alkenyl and acyl chain composition were plotted, split by sampling age (1, 6, 12 months in shades of red). For clarity, sidechains shown are restricted to those making up at least 1% of total PE-P in at least one sampling age.

FIG. 29 shows TG(O) composition in breast milk samples. Composition of TG(O) species present in breast milk samples from the BIS were calculated. TG(O) species composition were plotted, split by sampling age (1, 6, 12 months in shades of red). For clarity, TG(O) species are restricted to those making up at least 4% of total TG(O) in at least one sampling age.

FIG. 30 shows alkylglycerol composition in breast milk samples. Breast milk samples were saponified to hydrolyse the fatty acids from the lipid species and release the alkyl glycerol species from the TG(O) and DG(O) species. The alkyl glycerol (AG) species composition of the breast milk samples from the BIS are shown, split by sampling age (1, 6, 12 months).

FIG. 31 shows principal component analysis across all milk samples. Principal component analysis was performed on the lipidomic data from the BIS breast milk samples and the animal milk and formula milk samples. PCA scores plot (first 2 PCs), showing breast milk samples from each sampling age (1 month, red circles; 6 months, green circles; 12 months, blue circles), animal milk (open squares) and formula (open diamonds) (upper panel). Enlargement of the PCA scores plot (first 2 PCs), showing only the animal and formula milk samples (lower panel).

FIG. 32 shows PE(P) levels in breast milk, animal milk and formula. The concentration of total PE(P) across different milk samples. BM_01m: breast milk collected from 247 mothers when the infants were 1 month old; BM_06m: breast milk collected from 33 mothers when the infants were 6 months of age; BM_12m: breast milk collected from 33 mothers when the infants were 12 months of age; MF: milk formula.

Concentration is shown in pmol/mL.

FIG. 33 shows PE(P) alkenyl and acyl chain composition in breast milk, animal milk and formula. Alkenyl and acyl chain composition of the PE-P species present in breast milk, animal milk and formula milk samples were calculated. Alkenyl and acyl chain composition were plotted, split by sampling age (1, 6, 12 months in shades of red), animal milk and formula samples. For clarity, sidechains shown are restricted to those making up at least 1% of total PE-P in at least one sampling age.

FIG. 34 shows triacylglycerol and alkyl -diacylglycerol content in breast milk, animal milk and formula. The concentration (pmol/mL) of total TG (top panel), TG(O) middle panel) and the ratio of TG(O)/TG (lower panel) across different milk samples. BM_01m: breast milk collected from 247 mothers when the infants were 1 month old; BM_06m: breast milk collected from 33 mothers when the infants were 6 months of age; BM_12m: breast milk collected from 33 mothers when the infants were 12 months of age; MF=milk formula. Blue diamonds show the mean; white diamonds show the median.

FIG. 35 shows alkylglycerol content in saponified breast milk, animal milk and formula. The concentration (pmol/mL) of total alkylglycerol (AG) across different milk samples. BM_01m: breast milk collected from 247 mothers when the infants were 1 month old; BM_06m: breast milk collected from 33 mothers when the infants were 6 months of age; BM_12m: breast milk collected from 33 mothers when the infants were 12 months of age; MF=milk formula. Blue diamonds show the mean; white diamonds show the median.

FIG. 36 shows TG(O) and alkylglycerol composition in breast milk, animal milk and formula. Composition of TG(O) species present in breast milk, animal milk and formula milk samples were calculated. TG(O) species composition were plotted, split by sampling age (1, 6, 12 months), animal and formula. For clarity, TG(O) species are restricted to those making up at least 8% of total TG(O) in at least one sample type. The same milk samples were saponified to hydrolyse the fatty acids from the lipid species and release the alkyl glycerol species from the TG(O) and DG(O) species. The alkylglycerol (AG) species species composition of the breast milk, animal milk and formula milk samples are shown. Blue diamonds show the mean; white diamonds show the median.

FIG. 37 shows the relationship between breastfeeding, plasma lipid species and growth trajectories. Panel A—growth trajectories calculated for the infants in the BIS study. Panel B—reduction in % breastfeeding in the adverse growth trajectory (Red) relative to infants with average (Blue) and lower (green) BMI scores (all pairwise differences p<0.05). Panel C—linear regression of breastfeeding against 6-month infant plasma lipid concentration, adjusting for sex and weight. Beta-coefficients were transformed into % difference between breast fed vs formulae fed infants. Panel D- ordinal logistic regression of 6-month infant plasma lipid concentration against growth trajectories, adjusting for sex.

### DETAILED DESCRIPTION OF PARTICULAR EMBODIMENTS

Unless defined otherwise, all technical and scientific terms used herein have the same meaning as commonly understood by those of ordinary skill in the art to which the disclosure belongs.

As used herein the singular forms “a”, “an” and “the” include plural aspects unless the context clearly dictates otherwise. Thus, for example, reference to “a lipid species” includes a single lipid species, as well as two or more lipid species, reference to “the disclosure” includes single and multiple aspects of the disclosure and so forth.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. As used herein, the singular form “a”, “an” and “the” include singular and plural references unless the context indicates otherwise.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, or +/−5%, of the designated value.

The naming convention for lipids used here follows the guidelines established by the Lipid Maps Consortium and the shorthand notation of Liebisch et al. (Liebisch et al., Fahy et. al. (2009), Fahy et al. (2005)]. Phospholipids typically contain two fatty acid chains and in the absence of detailed characterisation are expressed as the sum composition of carbon atoms and double bonds (i.e. PC(38:6)). However, where an acyl chain composition has been determined the naming convention indicates this (i.e. PC(38:6) is changed to PC(16:0_22:6)). This is also extended into other lipid classes or subclasses. Species separated chromatographically but incompletely characterised were labelled with an (a) or (b), for example PC(P-17:0/20:4) (a) and (b) where (a) and (b) represent the elution order.

The present disclosure refers to lipid molecules using the numbering system X:Y. The number X represents the number of carbon atoms present in the chain.

In the context of alkylglycerols, alkyacylglycerols or alkyldiacylglycerols, the number Y represents the number of double bonds present in the chain. For example, an alkylglycerol numbered as 16:0 contains a hydrocarbon group having a 16 carbon chain with no double bonds. As a further example, an alkylglycerol numbered as 18:1 contains a hydrocarbon group having an 18 carbon chain with 1 double bond.

In the context of plasmalogens/plasmenyl phospholipids, the number Y in the first listed alkenyl chain (i.e. PE(P-X:Y/X:Y) represents the number of double bonds present in the alkenyl chain in addition to the vinyl ether group. For example, a plasmalogen numbered as PE(P-16:0/20:4) the 16:0 alkenyl group contains a hydrocarbon group having a 16 carbon chain with no double bonds other than the vinyl ether group (i.e. there is a double bond between the first 2 carbons and the remaining 14 carbons are saturated). As another example, a plasmalogen numbered as PE(P-18:1/20:4) the 18:1 alkenyl group contains a hydrocarbon group having an 18 carbon chain with 1 double bond in addition to the vinyl ether group (i.e. there is a double bond between the first 2 carbon atoms, and there is one other double bond between 2 carbons out of the remaining 16 carbons).

Where ether lipids contain one or more double bonds, the double bonds may be located at various positions in the hydrocarbon chains. For example, an alkylglycerol numbered as 18:1 may contain a mixture of species, e.g. with cis-n7 and cis-n9 double bonds. As another example, a plasmalogen (e.g PE(P)) numbered as 18:1 may contain a mixture of species, e.g. with cis-n7 and cis-n9 double bonds.

As used herein, the term “plasmanyl” shall be understood to refer to phospholipids having an ether bond in the sn-1 position to an alkyl group.

As used herein, the term “plasmenyl” shall be understood to refer to phospholipids having an ether bond in the sn-1 position to an alkenyl group. The plasmenyl phospholipids are referred to as “plasmalogens”.

A plasmalogen having a “15:0” alkenyl group is typically a molecule having an ether bond in the sn-1 position to an 15 carbon chain which contains a double bond between carbons 1 and 2 (i.e. typically a cis-vinyl ether group), and no other double bonds in the chain.

A plasmalogen having a “16:0” alkenyl group is typically a molecule having an ether bond in the sn-1 position to an 16 carbon chain which contains a double bond between carbons 1 and 2 (i.e. typically a cis-vinyl ether group), and no other double bonds in the chain.

A plasmalogen having a “17:0” alkenyl group is typically a molecule having an ether bond in the sn-1 position to an 17 carbon chain which contains a double bond between carbons 1 and 2 (i.e. typically a cis-vinyl ether group), and no other double bonds in the chain.

A plasmalogen having an “18:0” alkenyl group is typically a molecule having an ether bond in the sn-1 position to an 18 carbon chain which contains a double bond between carbons 1 and 2 (i.e. typically a cis-vinyl ether group), and no other double bonds in the chain.

A plasmalogen having an “18:1” alkenyl group is typically a molecule having an ether bond in the sn-1 position to an 18 carbon chain which contains a double bond between carbons 1 and 2 (i.e. typically a cis-vinyl ether group), and having one additional double bond, typically between carbons 7 and 8 (e.g. n7), between carbons 9 and 10 (e.g. n9), or between carbons 11 and 12 (e.g. n11), and typically a cis-double bond.

A plasmalogen having an “18:2” alkenyl group is typically a molecule having an ether bond in the sn-1 position to an 18 carbon chain which contains a double bond between carbons 1 and 2 (i.e. typically a cis-vinyl ether group), and having two additional double bonds, typically between carbons 9 and 10, and between carbons 11 and 12, and typically cis-double bonds.

A plasmalogen having a “20:0” alkenyl group is typically a molecule having an ether bond in the sn-1 position to an 15 carbon chain which contains a double bond between carbons 1 and 2 (i.e. typically a cis-vinyl ether group), and no other double bonds in the chain.

A plasmalogen having an “20:1” alkenyl group is typically a molecule having an ether bond in the sn-1 position to a 20 carbon chain which contains a double bond between carbons 1 and 2 (i.e. typically a cis-vinyl ether group), and having one additional double bond, typically between carbons 7 and 8 or between carbons 9 and 10, and typically a cis-double bond.

A plasmalogen having an “18:2” acyl alkenyl group is typically a molecule having an ester bond in the sn-2 position to an 18 carbon chain which has two double bonds, typically between carbons 9 and 10, and between carbons 11 and 12, and typically cis-double bonds.

A plasmalogen having a “20:4” acyl alkenyl group is typically a molecule having a ester bond in the sn-2 position to a 20 carbon chain which has four double bonds, typically between carbons 5 and 6, carbons 8 and 9, carbons 11 and 12, and carbons 14 and 15, and typically cis-double bonds.

A plasmalogen having a “22:6” acyl alkenyl group is typically a molecule having an ester bond in the sn-2 position to a 22 carbon chain which has six double bonds, typically between carbons 4 and 5, carbons 7 and 8, carbons 10 and 11, carbons 13 and 14, carbons 16 and 17, and carbons 19 and 20, and typically cis-double bonds.

As used herein, “acyl” refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C1-C24acyl- group include acetyl, benzoyl-, nicotinoyl-, propionyl-, isobutyryl-, oxalyl-, and the like. Lower-acyl refers to acyl groups containing one to four carbons. An acyl group can be unsubstituted or substituted, for example with one or more groups selected from halogen, —OH, —NH₂, —CN, —OC1-4alkyl and —CO₂H. Additional examples or generally applicable substituents are illustrated by the specific compounds described herein.

The term “aliphatic” as used herein, includes saturated, unsaturated, straight chain (i.e., unbranched), or branched, aliphatic hydrocarbons, which are optionally substituted with one or more functional groups. In some embodiments, the aliphatic may contain one or more functional groups such as double bond, triple bond, or a combination thereof. As will be appreciated by one of ordinary skill in the art, “aliphatic” is intended herein to include, but is not limited to, alkyl, alkenyl, alkynyl, or acyl moieties. Thus, as used herein, the term “alkyl” includes straight and branched saturated groups. An analogous convention applies to other generic terms such as “alkenyl”, “alkynyl”, “acyl” and the like. Furthermore, as used herein, the terms “alkyl”, “alkenyl”, “alkynyl”, “acyl” and the like encompass both substituted and unsubstituted groups.

As used herein, “alkenyl” refers to a straight or branched chain hydrocarbon containing, for example, from 2 to 30 carbons and containing at least one carbon-carbon double bond. In some embodiments, the alkenyl group contains 10 to 25, 14 to 22, or 16 to 20 carbon atoms. In some embodiments, the alkenyl group contains 15, 16, 17, 18, 19 or 20 carbon atoms. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, 3-undecenyl, 4-dodecenyl, 4-tridecenyl, 9-tetradecenyl, 8-pentadecenyl, 5-hexadecenyl, 8-heptadecenyl, 9-octadecenyl, 9-nonadecenyl and the like. Additional examples or generally applicable substituents are illustrated by the specific compounds described herein.

As used herein, “alkyl” refers to a straight or branched chain hydrocarbon containing, for example, from 1 to 30 carbon atoms. In some embodiments, the alkyl group contains 10 to 25, 14 to 22, or 16 to 20 carbon atoms. In some embodiments, the alkyl group contains 15, 16, 17, 18, 19 or 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, noctyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and the like. Additional examples or generally applicable substituents are illustrated by the specific compounds described herein.

As used herein, “acyl alkenyl” refers to a straight or branched chain hydrocarbon containing, for example, from 2 to 30 carbons and containing at least one carbon-carbon double bond, which is covalently bonded to an acyl group. The use of nomenclature 22:6 or 18:2 and the like in the context of an acyl alkenyl group refers to an acyl alkenyl group having 22 carbons or 18 carbons respectively, and having 6 or 2 double bonds respectively. An example of an acyl alkenyl group is:

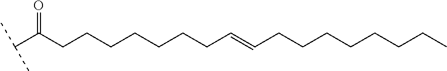

Acyl alkenyl groups may be present in species such as alkylacylglycerols or alkyldiacylglycerols (as an acyl group), or as an acyl group in plasmanyl- or plasmenyl-phospholipids. Typically, when present in those species, there is no double bond between the carbons which are α- and β- to the acyl group.

As used herein, “acyl alkyl” refers to a straight or branched chain hydrocarbon containing, for example, from 1 to 30 carbons, which is covalently bonded to an acyl group. The use of nomenclature 22:0 or 18:0 and the like in the context of an acyl alkyl group refers to an acyl alkyl group having 22 carbons or 18 carbons respectively. An example of an acyl alkyl group is:

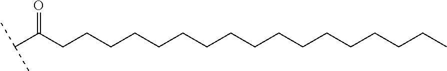

It will also be recognized that the compounds described herein may possess asymmetric centres and are therefore capable of existing in more than one stereoisomeric form. The disclosure thus also relates to compounds in substantially pure isomeric form at one or more asymmetric centres e.g., greater than 90% ee, such as 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

The present disclosure relates to derivatives of glycerol. Whilst glycerol is achiral, derivatives are typically chiral. Typically the glycerol utilised will have a stereochemical configuration corresponding to that found in nature. In some embodiments, the glycerol derivatives utilised have the following stereochemical configuration:

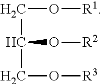

As referred to herein, the term “alkylglycerol” means a compound of Formula (I) in which the R¹ group is a hydrocarbon chain, the R² and R³ groups are each hydrogen.

Although the term “alkyl”glycerol is used, it will be understood by those of skill in the art that the term encompasses species with hydrocarbon groups at the R¹ position which include unsaturation in the hydrocarbon chain. However, an alkylglycerol does not contain a double bond between carbons 1 and 2 of the hydrocarbon chain, e.g. proximal to the ether linkage.

An alkylglycerol having a “16:0” group is typically a molecule having an ether bond in the sn-1 position to a 16 carbon saturated hydrocarbon chain, and no double bonds in the chain.

An alkylglycerol having an “18:0” group is typically a molecule having an ether bond in the sn-1 position to an 18 carbon saturated hydrocarbon chain, and no double bonds in the chain.

An alkylglycerol having an “18:1” group is typically a molecule having an ether bond in the sn-1 position to an 18 carbon hydrocarbon chain, which contains one double bond, typically between carbons 9 and 10 and typically a cis-double bond.

As referred to herein, the term “alkylacylglycerol” means a compound of Formula (I) in which the R¹ group is a hydrocarbon chain, one of the R² and R³ groups is hydrogen, and the other of the R² and R³ groups is an acyl group, either an acyl alkyl group or an acyl alkenyl group. Although the term “alkyl”acylglycerol is used, it will be understood by those of skill in the art that the term encompasses species with hydrocarbon groups at the R¹ position which include unsaturation in the hydrocarbon chain. However, an alkylacylglycerol does not contain a double bond between carbons 1 and 2 of the R¹ hydrocarbon chain, e.g. proximal to the ether linkage.

As referred to herein, the term “alkyldiacylglycerol” means a compound of Formula (I) in which the R¹ group is a hydrocarbon chain, and the R² and R³ groups are acyl groups, either acyl alkyl or acyl alkenyl. Although the term “alkyl”diacylglycerol is used, it will be understood by those of skill in the art that the term encompasses species with hydrocarbon groups at the R¹ position which include unsaturation in the hydrocarbon chain. However, an alkyldiacylglycerol does not contain a double bond between carbons 1 and 2 of the R¹ hydrocarbon chain, e.g. proximal to the ether linkage.

The term “extracted” with reference to a particular composition or substance refers to a composition or substance extracted from a natural source, including organisms and parts thereof. For example, lipids or oils extracted from a natural source, refer to lipids or oil that have been separated from other cellular materials, such as the natural source in which the lipid or oil was synthesized. Extracted lipids or oils are obtained through a wide variety of methods, the simplest of which involves physical means alone. For example, mechanical crushing using various press configurations (e.g. screw, expeller, piston, bead beaters, etc.) can separate lipids or oils from cellular materials. Alternately, lipid or oil extraction can occur via treatment with various organic solvents (e.g., hexane), via enzymatic extraction, via osmotic shock, via ultrasonic extraction, via supercritical fluid extraction (e.g., CO₂ extraction), via saponification and via combinations of these methods.

The term “BMI” refers to body mass index, and is calculated by dividing the weight of an individual in kg by their height in metres squared.

Reference to a “tissue” herein means any tissue such as blood, plasma, liver, heart, brain, adipose tissue, lymph, muscle.

The compositions are for in vivo maintenance of ether lipids at levels and/or ratios associated with a non-disease state, or wherein the composition is for in vivo modification of ether lipids towards levels and/or ratios associated with a non-disease state. In some embodiments, the compositions are for maintenance or modification of ether lipid levels and/or ratios in a tissue such as blood, plasma, liver, heart, brain, adipose tissue, lymph, muscle. In some embodiments, the compositions are for maintenance or modification of ether lipid levels and/or ratios in blood. In some embodiments, the compositions are for maintenance or modification of ether lipid levels and/or ratios in plasma. In some embodiments, the compositions are for maintenance of ether lipid levels and/or ratios. In some embodiments, the compositions are for modification of ether lipid levels and/or ratios.

As described in the present application, the composition of plasmalogen and other ether lipid alkenyl and acyl chains is tightly regulated and tissue specific. In one embodiment, therefore the present application provides compositions comprising a mixture of two or more ether lipid molecules suitable for maintaining or modulating the ether lipid composition of a particular tissue in a subject.

Reference to “two or more”, incudes 2, 3, 4, 5, 6, 7, 8, 9 or 10 or more ether lipids.

The proportion of the composition in a product is between about 0.001% to 80%, 0.01% to 70%, 0.1% and about 60%, between about 0.2% and about 50%, between about 6% and about 30%, between 1% and about 20%, between about 30% and about 60%, about 45% to about 60%, about 30%, or between about 15% and about 30% (as a percentage of the lipid class plus or minus 2 standard deviations).

### Ether Lipids and Compositions

The present inventors have found that ether lipids such as plasmanyl-phospholipids and plasmenyl-phospholipids (plasmalogens) have in vivo profiles which are associated with healthy state, and which are associated with conditions such as diabetes. It has also been found that the in vivo ether lipid profile can be affected by the administration of compositions containing ether lipids to subjects.

The compositions of the present disclosure find use in maintaining in vivo ether lipid levels at levels and/or ratios associated with a non-disease state, and/or in modifying in vivo ether lipid levels towards levels and/or ratios which are associated with a non-disease state.

Examples of ether phospholipids which the composition finds use in maintaining or modifying in vivo levels and/or ratios of include alkyl glycerols alkyl acyl glycerols (a compound which is an ether derivable from a glycerol alcohol and an alkyl alcohol, and which has an acyl group derivable from another glycerol alcohol and an acid) alkyl diacyl glycerols, and ether phospholipids such as plasmanyl-phospholipids and plasmenyl-phospholipids. In some embodiments, the composition is for in vivo maintenance or in vivo modification of plasmanyl- and/or plasmenyl-phospholipid levels and/or ratios. In some embodiments, the plasmanyl- and/or plasmenyl-phospholipids include those having phosphatidylcholine and/or phosphatidylethanolamine groups. In some embodiments, the composition is for in vivo maintenance or in vivo modification of plasmenyl-phospholipid (plasmalogen) levels and/or ratios. In some embodiments, the composition is for in vivo maintenance and/or modification of phosphatidylethanolamine plasmenyl-phospholipid (plasmalogen) levels and/or ratios.

Examples of plasmanyl-phospholipids, plasmenyl-phospholipids, and related lipidic species, and their abbreviations, are set out in the table below:

| Description | Abbreviation |
|---|---|
| Alkenylphosphatidylcholine | PC(P) |
| Alkenylphosphatidylethanolamine | PE(P) |
| Alkyl phosphatidylcholine | PC(O) |
| Alkyl phosphatidylethanolamine | PE(O) |
| Lysoalkylphosphatidylcholine | LPC(O) |
| Alkylglycerol | AG |
| Diacylglycerol | DG |
| Triacylglycerol | TG |
| Alkyl-diacylglycerol | TG(O) |
| Alkyl-acylglycerol | DG(O) |
| Fatty acids | FA |

The inventors have identified that healthy subjects tend to have a plasmanyl-phospholipid and plasmenyl-phospholipid profile in which certain alkenyl ether and alkyl ether groups are present. For example, a high proportion of ether lipids (i.e. plasmanyl- and/or plasmenyl-phospholipids) having 18:1 alkenyl ether groups, 18:0 alkyl ether groups and 16:0 alkyl ether groups were found in the group of healthy subjects. In particular, a high proportion of plasmalogens having 18:1 alkenyl ether groups, 18:0 alkyl ether groups and 16:0 alkyl ether groups were found in the group of healthy subjects.

Accordingly, in some embodiments, the composition is for in vivo maintenance or modification of ether lipids (e.g. plasmanyl- and/or plasmenyl-phospholipids) having 18:0 alkyl ether groups and 18:1 alkenyl ether groups. In some embodiments, the composition is for in vivo maintenance or modification of levels of plasmalogens having 18:0 ether groups and 18:1 ether groups. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids (e.g. plasmanyl- and/or plasmenyl-phospholipids) have a molar ratio of 18:0 alkyl ether groups to 18:1 alkenyl ether groups of from 1.2:1 to 2.5:1, from 1.5:1 to 2.1:1, or about 1.7:1. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids (e.g. plasmanyl- and/or plasmenyl-phospholipids) have a molar percent of 18:0 alkyl ether groups in the range of from 32.6% to 45.8%, and a molar percent of 18:1 alkenyl ether groups in the range of from 18.6% to 27.9%; for example having a molar percent of 18:0 alkyl ether groups in the range of from 32.6% to 45.8%, or having a molar percent of 18:0 alkyl ether groups of about 39.2%, and a molar percent of 18:1 alkenyl ether groups of about 23.3%.

In some embodiments, the composition is for in vivo maintenance of ether lipids at or in vivo modification of ether lipids towards an in vivo plasmalogen lipid profile in which the ether lipids have a molar ratio of 18:0 ether groups to 18:1 ether groups of from 1.2:1 to 2.5:1, from 1.5:1 to 2.1:1, or about 1.7:1. In some embodiments, the composition is for in vivo maintenance of ether lipids at or in vivo modification of ether lipids towards an in vivo plasmalogen lipid profile in which the ether lipids have a molar percent of 18:0 ether groups in the range of from 32.6% to 45.8%, and a molar percent of 18:1 ether groups in the range of from 18.6% to 27.9%; for example having a molar percent of 18:0 ether groups in the range of from 32.6% to 45.8%, or having a molar percent of 18:0 ether groups of about 39.2%, and a molar percent of 18:1 ether groups of about 23.3%.

In some embodiments, the composition is for in vivo maintenance or modification of ether lipids (e.g. plasmanyl- and/or plasmenyl-phospholipids) having 18:1 alkenyl ether groups and 16:0 alkyl ether groups. In some embodiments, the composition is for in vivo maintenance or modification of levels of plasmalogens having 18:1 ether groups and 16:0 ether groups. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipids) profile in which the ether lipids (e.g. plasmanyl- and/or plasmenyl-phospholipids) have a molar ratio of 18:1 alkenyl ether groups to 16:0 alkyl ether groups in the range of from 0.5:1 to 1:1, from 0.6:1 to 0.9:1, or about 0.72:1. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids (e.g. plasmanyl- and/or plasmenyl-phospholipids) have a molar percent of 18:1 alkenyl ether groups in the range of from 18.6% to 27.9%, and a molar percent of 16:0 alkenyl ether groups in the range of from 26.8% to 37.4%, for example having a molar percent of 18:1 alkenyl ether groups of about 23.3% and a molar percent of 16:0 alkyl ether groups of about 32.1%.

In some embodiments, the composition is for in vivo maintenance of ether lipids at or in vivo modification of ether lipids towards an in vivo plasmalogen lipid profile in which the ether lipids have a molar ratio of 18:1 ether groups to 16:0 ether groups of from 0.5:1 to 1:1, from 0.6:1 to 0.9:1, or about 0.72:1. In some embodiments, the composition is for in vivo maintenance of ether lipids at or in vivo modification of ether lipids towards an in vivo plasmalogen lipid profile in which the ether lipids have a molar percent of 18:1 ether groups in the range of from 18.6% to 27.9%, and a molar percent of 16:0 ether groups in the range of from 26.8% to 37.4%, for example having a molar percent of 18:1 ether groups of about 23.3% and a molar percent of 16:0 ether groups of about 32.1%.

In some embodiments, the composition is for in vivo maintenance or modification of ether lipids (e.g. plasmanyl- and/or plasmenyl-phospholipids) having 18:0 alkyl ether groups and 16:0 alkyl ether groups. In some embodiments, the composition is for in vivo maintenance or modification of levels of plasmalogens having 18:0 ether groups and 16:0 ether groups. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids have a molar ratio of 18:0 alkyl ether groups to 16:0 alkyl ether groups in the range of from 0.9:1 to 1.7:1, from 1:1 to 1.5:1, or about 1.22:1. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids (e.g. plasmanyl- and/or plasmenyl-phospholipids) have a molar percent of 18:0 alkyl ether groups in the range of from 32.6% to 45.8%, and a molar percent of 16:0 alkyl ether groups in the range of from 26.8% to 37.4%; or having a molar percent of 16:0 alkyl ether groups of about 32.1% and a molar percent of 18:0 alkyl ether groups of about 39.2%.

In some embodiments, the composition is for in vivo maintenance of ether lipids at or in vivo modification of ether lipids towards an in vivo plasmalogen lipid profile in which the ether lipids have a molar ratio of 18:0 ether groups to 16:0 ether groups of from 0.9:1 to 1.7:1, from 1:1 to 1.5:1, or about 1.22:1. In some embodiments, the composition is for in vivo maintenance of ether lipids at or in vivo modification of ether lipids towards an in vivo plasmalogen lipid profile in which the ether lipids have a molar percent of 18:0 ether groups in the range of from 32.6% to 45.8%, and a molar percent of 16:0 ether groups in the range of from 26.8% to 37.4%; or having a molar percent of 16:0 ether groups of about 32.1% and a molar percent of 18:0 ether groups of about 39.2%.

In some embodiments, the composition is for in vivo maintenance or modification of ether lipids (e.g. plasmanyl- and/or plasmenyl-phospholipids) having 18:1 alkenyl ether groups, 18:0 alkyl ether groups and 16:0 alkyl ether groups. In some embodiments, the composition is for in vivo maintenance or modification of levels of plasmalogens having 18:1 ether groups, 18:0 ether groups and 16:0 ether groups. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids (e.g. plasmanyl- and/or plasmenyl-phospholipids) having a molar ratio of 18:1 alkenyl ether groups to 18:0 alkyl ether groups to 16:0 alkyl ether groups of about 1:1.7:1.4. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids (e.g. plasmanyl- and/or plasmenyl-phospholipids) have a molar percent of 18:1 alkenyl ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%; or having a molar percent of 18:1 alkenyl ether groups of about 23.3%, a molar percent of 18:0 alkyl ether groups of about 39.2%, and a molar percent of 16:0 alkyl ether groups of about 32.1%.

In some embodiments, the composition is for in vivo maintenance of ether lipids at or in vivo modification of ether lipids towards an in vivo plasmalogen lipid profile in which the ether lipids have a molar ratio of 18:1 ether groups to 18:0 ether groups to 16:0 ether groups of about 1:1.7:1.4. In some embodiments, the composition is for in vivo maintenance of ether lipids at or in vivo modification of ether lipids towards an in vivo plasmalogen lipid profile in which the ether lipids have a molar percent of 18:1 ether groups in the range of from 18.6% to 27.9%, a molar percent of 18:0 ether groups in the range of from 32.6% to 45.8%, and a molar percent of 16:0 ether groups in the range of from 26.8% to 37.4%; or having a molar percent of 18:1 ether groups of about 23.3%, a molar percent of 18:0 ether groups of about 39.2%, and a molar percent of 16:0 ether groups of about 32.1%.

Ether lipids (i.e. plasmanyl- and/or plasmenyl-phospholipids) having 15:0 alkyl ether groups, 17:0 alkyl ether groups, 19:0 alkyl ether groups, 20:0 alkyl ether groups and 20:1 alkenyl ether groups were also identified as being present in the group of healthy subjects. The levels of those alkyl ether and alkenyl ether groups were lower than for 18:1 alkenyl ether, 18:0 alkyl ether and 16:0 alkyl ether groups.

In some embodiments, the composition is for in vivo maintenance or modification of ether lipids (e.g. plasmanyl- and/or plasmenyl-phospholipids) having one or more of 15:0 alkyl ether groups, 17:0 alkyl ether groups, 19:0 alkyl ether groups, 20:0 alkyl ether groups and 20:1 alkyl ether groups. In some embodiments, the composition is for in vivo maintenance or modification of levels of plasmalogens having one or more of 15:0 ether groups, 17:0 ether groups, 19:0 ether groups, 20:0 ether groups and 20:1 ether groups. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipids) profile in which the ether lipids (e.g. plasmanyl- and/or plasmenyl-phospholipids) have a molar percent of 15:0 alkyl ether groups in the range of from 0.2% to 1.19%, a molar percent of 17:0 alkyl ether groups in the range of from 1.5% to 3.3%, a molar percent of 19:0 alkyl ether groups in the range from 0.06% to 0.34%, a molar percent of 20:0 alkyl ether groups in the range of from 0.8 to 2.5%, and/or a molar percent of 20:1 alkenyl ether groups in the range of from 0 to 1.2%; or a molar percent of 15:0 alkyl ether groups of about 0.7%, a molar percent of 17:0 alkyl ether groups of about 2.4%, a molar percent of 19:0 alkyl ether groups of about 0.2%, a molar percent of 20:0 alkyl ether groups of about 1.6%, and/or a molar percent of 20:1 alkenyl ether groups of about 0.5%.

Ether lipids (e.g. plasmanyl- and/or plasmenyl-phospholipids) having acyl alkenyl groups such as 20:4 acyl alkenyl groups, 22:6 acyl alkenyl groups, and/or 18:2 acyl alkenyl groups, were also identified as being present in healthy subjects, in a high proportion of the acyl groups present as a whole.

In some embodiments, the composition is for in vivo maintenance or modification of ether lipids (e.g. plasmanyl- and/or plasmenyl-phospholipids) having 20:4 acyl alkenyl groups, 22:6 acyl alkenyl groups, and/or 18:2 acyl alkenyl groups. In some embodiments, the composition is for in vivo maintenance or modification of levels of plasmalogens having 20:4 acyl alkenyl groups, 22:6 acyl alkenyl groups, and/or 18:2 acyl alkenyl groups. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid profile (e.g. plasmanyl- and/or plasmenyl-phospholipids) in which the ether lipids (e.g. plasmanyl- and/or plasmenyl-phospholipids) have a molar ratio of 20:4 acyl alkenyl groups to 22:6 acyl alkenyl groups to 18:2 acyl alkenyl groups of about 3:1.2:1. In some embodiments, the composition is for in vivo maintenance of ether lipids (e.g. plasmanyl- and/or plasmenyl-phospholipids) at or in vivo modification of ether lipids (e.g. plasmanyl- and/or plasmenyl-phospholipids) towards an in vivo total ether lipid (e.g. plasmanyl- and/or plasmenyl-phospholipid) profile in which the ether lipids (e.g. plasmanyl- and/or plasmenyl-phospholipids) have acyl alkenyl groups in which the molar percent of 20:4 acyl alkenyl groups is in the range of from 31.3% to 52.5%, the molar percent of 22:6 acyl alkenyl groups is in the range of from 9.3% to 23.9%, and the molar percent of 18:2 acyl alkenyl groups is in the range of from 7.6% to 19.9%; or in which the molar percent of 20:4 acyl alkenyl groups is about 41.9%, the molar percent of 22:6 acyl alkenyl groups is about 16.7%, and the molar percent of 18:2 acyl alkenyl groups is about 13.8%.

In some embodiments, the composition is for in vivo maintenance of ether lipids at or in vivo modification of ether lipids towards an in vivo plasmalogen lipid profile in which the ether lipids have a molar ratio of 20:4 acyl alkenyl groups to 22:6 acyl alkenyl groups to 18:2 acyl alkenyl groups of about 3:1.2:1. In some embodiments, the composition is for in vivo maintenance of ether lipids at or in vivo modification of ether lipids towards an in vivo plasmalogen lipid profile in which the ether lipids have acyl alkenyl groups in which the molar percent of 20:4 acyl alkenyl groups is in the range of from 31.3% to 52.5%, the molar percent of 22:6 acyl alkenyl groups is in the range of from 9.3% to 23.9%, and the molar percent of 18:2 acyl alkenyl groups is in the range of from 7.6% to 19.9%; or in which the molar percent of 20:4 acyl alkenyl groups is about 41.9%, the molar percent of 22:6 acyl alkenyl groups is about 16.7%, and the molar percent of 18:2 acyl alkenyl groups is about 13.8%.

The composition comprises a mixture of ether lipids of Formula (I):

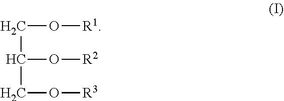

Ether lipids of formula (I) include alkyl glycerols, alkenyl glycerols, alkyl acyl glycerols, alkenyl acyl glycerols, alkyl diacyl glycerols, alkenyl diacyl glycerols, and ether phospholipids such as plasmanyl-phospholipids and plasmenyl-phospholipids.

In some embodiments, the ether lipids of formula (I) are selected from the group consisting of alkyl glycerols, alkenyl glycerols, alkyl acyl glycerols, alkenyl acyl glycerols, alkyl diacyl glycerols and, alkenyl diacyl glycerols (i.e. in which case R² is hydrogen or

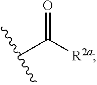

and R³ is hydrogen or

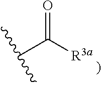

In some embodiments, the ether lipids of formula (I) are alkyl glycerols (i.e. in which case R² and R³ are hydrogen). As discussed above, alkylglycerols are lipids with a glycerol backbone, to which fatty acid or fatty acid derivatives are coupled by means of an ether bond instead of the ester bond that characterizes most mono-, di- and tri-glycerols and related phospholipids (see, e.g., U.S. Pat. No. 6,121,245, which is incorporated herein by reference in its entirety).

In some embodiments the mixture of ether lipids of Formula (I) is a mixture comprising alkylglycerols, and the ether lipids which are to be maintained or modified in vivo are plasmanyl-phospholipids and/or plasmenyl-phospholipids.

In the ether lipids of Formula (I), R¹ is an alkyl or alkenyl group. In some embodiments, the composition comprises ether lipid molecules of Formula (I) in which le is C₁₀₋₂₄alkyl and/or C₁₀₋₂₄alkenyl groups. In some embodiments, the composition comprises ether lipid molecules of Formula (I) in which R¹ is C₁₅₋₂oalkyl and/or C₁₅₋₂₀alkenyl groups. In some embodiments, the composition comprises ether lipid molecules having an 18:0 alkyl R¹ group, ether lipid molecules having an 18:1 alkenyl R¹ group, ether lipid molecules having a 16:0 alkyl R¹ group, ether lipid molecules having a 15:0 alkyl R¹ group, ether lipid molecules having a 17:0 alkyl R¹ group, ether lipid molecules having a 19:0 alkyl R¹ group, ether lipid molecules having a 20:0 alkyl R¹ group, and/or ether lipid molecules having a 20:1 alkenyl R¹ group. In some embodiments, the composition comprises ether lipid molecules having an 18:0 alkyl R¹ group, ether lipid molecules having an 18:1 alkenyl R¹ group, and/or ether lipid molecules having a 16:0 alkyl R¹ group. Examples of ether lipid molecules of Formula (I) include batyl alcohol, chimyl alcohol and selachyl alcohol.

In some embodiments, the composition comprises ether lipid molecules having an 18:0 alkyl R¹ group, and ether lipid molecules having an 18:1 alkenyl R¹ group. In some embodiments, the composition comprises ether lipids having a molar ratio of 18:0 alkyl le groups to 18:1 alkenyl R¹ groups in the range of from 1.2:1 to 2.5:1, from 1.5:1 to 2.1:1, or about 1.7:1. In some embodiments, ether lipids having an 18:0 alkyl R¹ group and ether lipids having an 18:1 alkenyl R¹ group together comprise at least 50%, at least 75%, at least 80%, at least 85%, at least 90%, at least 95% or at least 98% of the ether lipids in the composition.

In some embodiments, the composition comprises ether lipid molecules having an 18:1 alkenyl R¹ group, and ether lipid molecules having a 16:0 alkyl R¹ group. In some embodiments, the composition comprises ether lipids having a molar ratio of 18:1 alkenyl R¹ groups to 16:0 alkyl R¹ groups in the range of from 0.5:1 to 1:1, from 0.6:1 to 0.9:1, or about 0.72:1. In some embodiments, ether lipids having an 18:1 alkenyl R¹ group and ether lipids having an 16:0 alkyl R¹ group together comprise at least 50%, at least 75%, at least 80%, at least 85%, at least 90%, at least 95% or at least 98% of the ether lipids in the composition.

In some embodiments, the composition comprises ether lipid molecules having an 18:0 alkyl R¹ group, and ether lipid molecules having a 16:0 alkyl R¹ group. In some embodiments, the composition comprises ether lipids having a molar ratio of 18:0 alkyl R¹ groups to 16:0 alkyl R¹ groups in the range of from 0.9:1 to 1.7:1, from 1:1 to 1.5:1, or about 1.22:1. In some embodiments, ether lipids having an 18:0 alkyl R¹ group and ether lipids having an 16:0 alkyl R¹ group together comprise at least 50%, at least 75%, at least 80%, at least 85%, at least 90%, at least 95% or at least 98% of the ether lipids in the composition.

In some embodiments, the composition comprises ether lipid molecules having an 18:1 alkenyl R¹ group, ether lipid molecules having an 18:0 alkyl R¹ group, and ether lipid molecules having a 16:0 alkyl R¹ group. In some embodiments, the composition comprises ether lipid molecules having an 18:1 alkenyl R¹ group, ether lipid molecules having an 18:0 alkyl R¹ group, and ether lipid molecules having a 16:0 alkyl R¹ group, and wherein the molar ratio of 16:0 alkyl R¹ groups to 18:1 alkenyl R¹ groups is at least 1:1. In some embodiments, the composition comprises ether lipid molecules having an 18:1 alkenyl R¹ group, ether lipid molecules having an 18:0 alkyl R¹ group, and ether lipid molecules having a 16:0 alkyl R¹ group, and wherein the molar ratio of 16:0 alkyl R¹ groups to 18:1 alkenyl R¹ groups to 18:0 alkyl R¹ groups is in the range of from 0.7:1:1.3 to 1.3:1:0.7.

In some embodiments, composition comprises ether lipid molecules having an 18:1 alkenyl R¹ group, ether lipid molecules having a 18:0 alkyl R¹ group, and ether lipid molecules having a 16:0 alkyl R¹ group, in which the molar ratio of 18:0 alkyl R¹ groups to 18:1 alkenyl R¹ groups is in the range of from 1.2:1 to 2.5:1, from 1.5:1 to 2.1:1, or about 1.7:1; in which the molar ratio of 18:0 alkyl R¹ groups to 16:0 alkyl R¹ groups in the range of from 0.9:1 to 1.7:1, from 1:1 to 1.5:1, or about 1.22:1; and/or in which the molar ratio of 18:1 alkenyl R¹ groups to 16:0 alkyl R¹ groups is in the range of from 0.5:1 to 1:1, from 0.6:1 to 0.9:1, or about 0.72:1. In some embodiments, the composition comprises ether lipids having a molar ratio of 18:1 alkenyl R¹ groups to 18:0 alkyl R¹ groups to 16:0 alkyl R¹ groups of about 1:1.7:1.4.

In some embodiments, ether lipids having an 18:1 alkenyl R¹ group, ether lipids having an 18:0 alkyl R¹ group, and ether lipids having an 16:0 alkyl R¹ group together comprise at least 50%, at least 75%, at least 80%, at least 85%, at least 90%, at least 95% or at least 98% of the ether lipids in the composition.

In some embodiments, the composition additionally comprises ether lipids having R¹ groups selected from the group consisting of 15:0 alkyl, 17:0 alkyl, 19:0 alkyl, 20:0 alkyl, and 20:1 alkenyl. In some embodiments, the composition comprises ether lipid molecules having a 18:0 alkyl R¹ group, and ether lipid molecule having a 15:0 alkyl R¹ group, wherein the molar ratio is in the range of from 50:1 to 60:1. In some embodiments, the composition comprises ether lipid molecules having a 18:0 alkyl R¹ group, and ether lipid molecule having a 17:0 alkyl R¹ group, wherein the molar ratio is in the range of from 20:1 to 12:1. In some embodiments, the composition comprises ether lipid molecules having a 18:0 alkyl R¹ group, and ether lipid molecule having a 19:0 alkyl R¹ group, wherein the molar ratio is in the range of from 100:1 to 300:1. In some embodiments, the composition comprises ether lipid molecules having a 18:0 alkyl R¹ group, and ether lipid molecule having a 20:0 alkyl R¹ group, wherein the molar ratio is in the range of from 20:1 to 30:1. In some embodiments, the composition comprises ether lipid molecules having a 18:0 alkyl R¹ group, and ether lipid molecule having a 20:1 alkyl R¹ group, wherein the molar ratio is in the range of from 50:1 to 100:1.

The inventors have identified that certain alkyl ether and alkenyl ether groups are associated with likelihood of diabetes, and/or likelihood of incident diabetes.

In some embodiments, the composition is for increasing the proportion of 15:0, 17:0, 18:0 and/or 19:0 alkyl ether groups present in in vivo ether lipids. In some embodiments, the composition comprises one or more of ether lipid molecules having a 15:0 alkyl R¹ group, ether lipid molecules having a 17:0 alkyl R¹ group, ether lipid molecules having an 18:0 alkyl R¹ group and ether lipid molecules having a 19:0 alkyl R¹ group. In some embodiments, the composition comprises one or more of ether lipid molecules having a 15:0 alkyl R¹ group, which forms at least 5%, at least 10%, at least 20%, at least 30%, at least 40%, or at least 50% of the ether lipid molecules present in the composition. In some embodiments, the composition comprises one or more of ether lipid molecules having a 17:0 alkyl R¹ group, which forms at least 5%, at least 10%, at least 20%, at least 30%, at least 40%, or at least 50% of the ether lipid molecules present in the composition. In some embodiments, the composition comprises one or more of ether lipid molecules having an 18:0 alkyl R¹ group, which forms at least 5%, at least 10%, at least 20%, at least 30%, at least 40%, or at least 50% of the ether lipid molecules present in the composition. In some embodiments, the composition comprises one or more of ether lipid molecules having a 19:0 alkyl R¹ group, which forms at least 5%, at least 10%, at least 20%, at least 30%, at least 40%, or at least 50% of the ether lipid molecules present in the composition.

In some embodiments, the composition is for decreasing the proportion of 16:0 and/or 20:0 alkyl ether groups present in in vivo ether lipids. In some embodiments, the composition is free or substantially free of ether lipid molecules containing 16:0 alkyl R¹ groups. In some embodiments, the composition is free or substantially free of ether lipid molecules containing 20:0 alkyl R¹ groups.

The ether lipid molecules of Formula (I) have R² and R³ groups.

In some embodiments, the composition comprises ether lipids wherein R² and R³ is hydrogen (e.g. alkylglycerols).

In some embodiments, the composition comprises ether lipids in which R² is hydrogen and R³ is

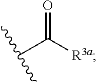

and R^{3a} is selected from the group consisting of a saturated alkyl hydrocarbon, a monounsaturated alkenyl hydrocarbon and a polyunsaturated alkenyl hydrocarbon (e.g. alkyl acyl glycerol).

In some embodiments, the composition comprises ether lipids in which R³ is hydrogen and R² is

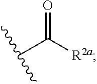

and R^{2a} is selected from the group consisting of a saturated alkyl hydrocarbon, a monounsaturated alkenyl hydrocarbon and a polyunsaturated alkenyl hydrocarbon (e.g. alkyl acyl glycerol).

In some embodiments, the composition comprises ether lipids in which R² is:

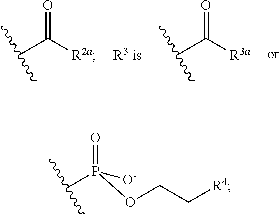

wherein R^{2a} is selected from the group consisting of a saturated alkyl hydrocarbon, a monounsaturated alkenyl hydrocarbon and a polyunsaturated alkenyl hydrocarbon; R^{3a} is selected from the group consisting of a saturated alkyl hydrocarbon, a monounsaturated alkenyl hydrocarbon and a polyunsaturated alkenyl hydrocarbon; and R⁴ is —N(Me)3⁺ or —NH₃⁺ (e.g. alkyl diacyl glycerol, PC or PE plasmanyl- or plasmenyl-phospholipid).

In embodiments where an alkylglycerol is administered, or where an alkylacyl glycerol is administered, or where an alkyldiacylglycerol is administered, references to an alkyl or alkenyl R¹ group having the numbering X:Y means that the group has X carbons, and has Y double bonds.

In embodiments where a plasmalogen is administered, references to an alkyl or alkenyl R¹ group having the numbering X:Y means that the group has X carbons, and has Y double bonds in addition to the vinyl ether.

In some embodiments, the composition comprises ether lipid molecules having a 20:4 acyl alkenyl R² and/or R³ group, ether lipids having a 22:6 acyl alkenyl R² and/or R³ group, ether lipids having an 18:2 acyl alkenyl R² and/or R³ group, ether lipids having a 18:1 acyl alkenyl R² and/or R³ group, ether lipids having a 18:3 acyl alkenyl R² and/or R³ group, ether lipids having a 20:3 acyl alkenyl R² and/or R³ group, ether lipids having a 20:5 acyl alkenyl R² and/or R³ group, ether lipids having a 22:4 acyl alkenyl R² and/or R³ group, and/or ether lipids having a 22:5 acyl alkenyl R² and/or R³ group. In some embodiments, the composition comprises ether lipid molecules having a 20:4 acyl alkenyl R² and/or R³ group, ether lipids having a 22:6 acyl alkenyl R² and/or R³ group, and/or ether lipids having an 18:2 acyl alkenyl R² and/or R³ group.

In some embodiments, the composition comprises ether lipid molecules having a 20:4 acyl alkenyl R² and/or R³ group, ether lipids having a 22:6 acyl alkenyl R² and/or R³ group, and ether lipids having an 18:2 acyl alkenyl R² and/or R³ group.

In some embodiments, the composition comprises ether lipid molecules having a 20:4 acyl alkenyl R² and/or R³ group, ether lipids having a 22:6 acyl alkenyl R² and/or R³ group, and ether lipids having an 18:2 acyl alkenyl R² and/or R³ group, wherein the molar ratio of 20:4 acyl alkenyl groups to 22:6 acyl alkenyl groups to 18:2 acyl alkenyl groups is about 3:1.2:1.

The inventors have identified that certain acyl alkenyl groups are associated with likelihood of diabetes, and/or likelihood of incident diabetes.

In some embodiments, the composition is for decreasing the proportion of 18:1 and/or 20:3 acyl alkenyl ether groups present in in vivo ether lipids. In some embodiments, the composition is free or substantially free of ether lipid molecules containing 18:1 acyl alkenyl R² and/or R³ groups. In some embodiments, the composition is free or substantially free of ether lipid molecules containing 20:3 acyl alkenyl R² and/or R³ groups.

### Compositions for Infants

It has been identified that healthy infant subjects tend to have a plasmenyl-phospholipid profile in which certain ether groups are present. For example, a high proportion of plasmalogens (e.g. PE(P)) having 18:1 ether groups, 18:0 ether groups and 16:0 ether groups were found in the group of healthy infant subjects. Differences in infant plasma lipidome profile have been found to be associated with health and growth outcomes, e.g. in relation to risk of being overweight, obese or asthmatic. It has also been identified that breast milk has a different ether lipid profile, i.e. alkylglycerol profile, to animal milks or formula milks, and that the nature of infant diet is associated with a different plasma lipidome profile.

Accordingly, there is also provided a composition comprising a mixture of ether lipid molecules of Formula (I):

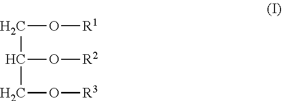

wherein
R¹ is an alkyl or alkenyl group;
R² is hydrogen or and

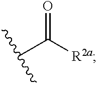

R³ is hydrogen,

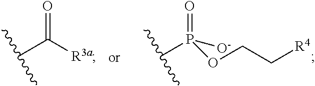

wherein

R^{2a} and R^{3a} are each an alkyl or alkenyl group; and

R⁴ is —N(Me)₃⁺ or —NH₃⁺.

wherein the composition is present in the form of a product which is a liquid infant formula milk, an infant formula milk powder, a supplement for addition to infant formula milk, a supplement for addition to infant food, or an infant dietary supplement.

The composition contains ether lipids so as to maintain or modify the plasmalogen ether lipid profile in vivo at or towards a healthy profile, for example it may be based on plasmalogen (eg. PE(P)) ether lipid profile identified in infant plasma.

In some embodiments, the composition comprises ether lipid molecules having an 18:0 R¹ group, and ether lipid molecules having an 18:1 R¹ group. In some embodiments, the composition is for in vivo maintenance of ether lipids at, or in vivo modification of ether lipids towards, an in vivo plasmalogen ether lipid (e.g. PE(P)) profile in which the ether lipids have a molar ratio of 18:0 ether groups to 18:1 ether groups of from 0.74:1 to 1.60:1, from 0.8:1 to 1.5:1, from 0.95:1 to 1.25:1, or about 1.1:1. In some embodiments, the composition is for in vivo maintenance of ether lipids at or in vivo modification of ether lipids towards an in vivo plasmalogen ether lipid (e.g. PE(P)) profile in which the ether lipids have a molar percent of 18:0 ether groups in the range of from 27.7% to 39.6%, and a molar percent of 18:1 ether groups in the range of from 24.7% to 37.4%, or have a molar percent of 18:0 ether groups in the range of from 28.8% to 38.3%, and a molar percent of 18:1 ether groups in the range of from 25.7% to 35.8%; for example having a molar percent of 18:0 ether groups in the range of from 31.8% to 35.8%, and a molar percent of 18:1 ether groups in the range of from 28.6% to 32.5%; or having a molar percent of 18:0 ether groups of about 34% (e.g. 33.9%), and a molar percent of 18:1 ether groups of about 31% (e.g. 30.7%). In some embodiments, the composition has ether lipids having a molar ratio of 18:0 ether groups to 18:1 ether groups of from 0.74:1 to 1.60:1, from 0.8:1 to 1.5:1, from 0.95:1 to 1.25:1, or about 1.1:1.

In some embodiments, the composition comprises ether lipid molecules having an 18:1 R¹ group, and ether lipid molecules having a 16:0 R¹ group. In some embodiments, the composition is for in vivo maintenance of ether lipids at or in vivo modification of ether lipids towards an in vivo plasmalogen ether lipid (e.g. PE(P)) profile in which the ether lipids have a molar ratio of 18:1 ether groups to 16:0 ether groups in the range of from 1.24:1 to 0.59:1, from 2:1 to 1.1:1, from 0.76:1 to 1:1 or about 1:1.2 (e.g. 1:1.16). In some embodiments, the composition is for in vivo maintenance of ether lipids at or in vivo modification of ether lipids towards an in vivo plasmalogen ether lipid (e.g. PE(P)) profile in which the ether lipids have a molar percent of 18:1 ether groups in the range of from 24.7% to 37.4%, and a molar percent of 16:0 ether groups in the range of from 30.1% to 41.7%, or have a molar percent of 18:1 ether groups in the range of from 25.7% to 35.8%, and a molar percent of 16:0 ether groups in the range of from 30.9% to 40.7%, or a molar percent of 18:1 ether groups in the range of from 28.6% to 32.5%, and a molar percent of 16:0 ether groups in the range of from 33.5% to 37.4%, for example having a molar percent of 18:1 ether groups of about 30.7% and a molar percent of 16:0 ether groups of about 35.5%. In some embodiments, the composition has ether lipids having a molar ratio of 18:1 ether groups to 16:0 ether groups in the range of from 1.24:1 to 0.59:1, from 2:1 to 1.1:1, from 0.76:1 to 1:1 or about 1:1.2 (e.g. 1:1.16).

In some embodiments, the composition comprises ether lipid molecules having an 18:0 R¹ group, and ether lipid molecules having a 16:0 R¹ group. In some embodiments, the composition is for in vivo maintenance of ether lipids at or in vivo modification of ether lipids towards an in vivo plasmalogen ether lipid (e.g. PE(P)) profile in which the ether lipids have a molar ratio of 18:0 ether groups to 16:0 ether groups in the range of from 0.66:1 to 1.3:1, from 1.25:1 to 1:1.45, from 0.85:1 to 1.1:1, or about 1:1 (e.g. 0.95:1). In some embodiments, the composition is for in vivo maintenance of ether lipids at or in vivo modification of ether lipids towards an in vivo plasmalogen ether lipid (e.g. PE(P)) profile in which the ether lipids have a molar percent of 18:0 ether groups in the range of from 27.7% to 39.6%, and a molar percent of 16:0 ether groups in the range of from 30.1 to 41.7%; or have a molar percent of 18:0 ether groups in the range of from 28.8% to 38.3%, and a molar percent of 16:0 ether groups in the range of from 30.9 to 40.7%; or have a molar percent of 18:0 ether groups in the range of from 31.8% to 35.8%, and a molar percent of 16:0 ether groups in the range of from 33.5% to 37.4%; or having a molar percent of 16:0 ether groups of about 35.5% and a molar percent of 18:0 ether groups of about 33.9%. In some embodiments, the composition has ether lipids having a molar ratio of 18:0 ether groups to 16:0 ether groups in the range of from 0.66:1 to 1.3:1, from 1.25:1 to 1:1.45, from 0.85:1 to 1.1:1, or about 1:1 (e.g. 0.95:1).

In some embodiments, the composition comprises ether lipid molecules having an 18:1 R¹ group, ether lipid molecules having an 18:0 R¹ group, and ether lipid molecules having a 16:0 R¹ group.

In some embodiments, the composition is for in vivo maintenance of ether lipids at or in vivo modification of ether lipids towards an in vivo plasmalogen ether lipid (e.g. PE(P)) profile in which the ether lipids have a molar ratio of 18:1 ether groups to 18:0 ether groups to 16:0 ether groups of about 0.9:1.0:1.05. In some embodiments, the composition is for in vivo maintenance of ether lipids at or in vivo modification of ether lipids towards an in vivo plasmalogen ether lipid (e.g. PE(P)) profile in which the ether lipids have a molar percent of 18:1 ether groups in the range of from 24.7% to 37.4%, a molar percent of 18:0 ether groups in the range of from 27.7% to 39.6%, and a molar percent of 16:0 ether groups in the range of from 30.1% to 41.7%; or have a molar percent of 18:1 ether groups in the range of from 25.7% to 35.8%, a molar percent of 18:0 ether groups in the range of from 28.8% to 38.3%, and a molar percent of 16:0 ether groups in the range of from 30.9% to 40.7%; or have a molar percent of 18:1 ether groups in the range of from 28.6% to 32.5%, a molar percent of 18:0 ether groups in the range of from 31.8% to 35.8%, and a molar percent of 16:0 ether groups in the range of from 33.5% to 37.4%; or have a molar percent of 18:1 ether groups of about 30.7%, a molar percent of 18:0 ether groups of about 33.9%, and a molar percent of 16:0 ether groups of about 35.5%. In some embodiments, the composition has ether lipids having molar ratio of 18:1 ether groups to 18:0 ether groups to 16:0 ether groups of about 0.9:1.0:1.05.

Ether lipids (e.g. plasmalogens, particularly PE(P)) having 16:0 ether groups, 18:2 ether groups, 20:0 ether groups and 20:1 ether groups were also identified as being present in the group of healthy subjects. The levels of those ether groups were lower than for 18:1 ether, 18:0 ether and 16:0 ether groups.

In some embodiments, the composition additionally comprises ether lipids having R¹ groups selected from the group consisting of 16:0, 18:2, 20:0 and 20:1.

As discussed above, the composition comprises a mixture of ether lipids of Formula (I):

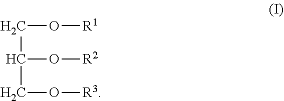

Ether lipids of formula (I) include alkyl glycerols, alkyl acyl glycerols, alkyl diacyl glycerols, and ether phospholipids such as plasmanyl-phospholipids and plasmenyl-phospholipids.

In some embodiments, the ether lipids of formula (I) are selected from the group consisting of alkyl glycerols, alkyl acyl glycerols, and alkyl diacyl glycerols (i.e. in which case R² is hydrogen or

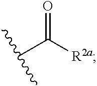

and R³ is hydrogen or

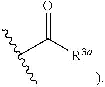

In some embodiments, the ether lipids of formula (I) are alkyl glycerols (i.e. in which case R² and R³ are hydrogen). As discussed above, alkylglycerols are lipids with a glycerol backbone, to which fatty acid or fatty acid derivatives are coupled by means of an ether bond instead of the ester bond that characterizes most mono-, di- and tri-glycerols and related phospholipids (see, e.g., U.S. Pat. No. 6,121,245, which is incorporated herein by reference in its entirety).

In some embodiments the mixture of ether lipids of Formula (I) is a mixture comprising alkylglycerols, and the ether lipids which are to be maintained or modified in vivo are plasmanyl-phospholipids.

In the ether lipids of Formula (I), R¹ is an alkyl or alkenyl group. In some embodiments, the composition comprises ether lipid molecules of Formula (I) in which R¹ is C₁₀₋₂₄alkyl and/or C₁₀₋₂₄alkenyl groups. In some embodiments, the composition comprises ether lipid molecules of Formula (I) in which R¹ is C₁₅₋₂oalkyl and/or C₁₅₋₂₀alkenyl groups. In some embodiments, the composition comprises ether lipid molecules having an 18:0 alkyl R¹ group, ether lipid molecules having an 18:1 alkenyl R¹ group, ether lipid molecules having a 16:0 alkyl R¹ group, ether lipid molecules having a 15:0 alkyl R¹ group, ether lipid molecules having a 17:0 alkyl R¹ group, ether lipid molecules having a 19:0 alkyl R¹ group, ether lipid molecules having a 20:0 alkyl R¹ group, and/or ether lipid molecules having a 20:1 alkenyl R¹ group. In some embodiments, the composition comprises ether lipid molecules having an 18:0 alkyl R¹ group, ether lipid molecules having an 18:1 alkenyl R¹ group, and/or ether lipid molecules having a 16:0 alkyl R¹ group. Examples of ether lipid molecules of Formula (I) include batyl alcohol, chimyl alcohol and selachyl alcohol.

In some embodiments, the composition may be based on the ether lipid profile (e.g. alkylglycerol ether lipid profile) identified in human breast milk.

In some embodiments, the composition comprises ether lipid molecules having an 18:0 R¹ group, and ether lipid molecules having an 18:1 R¹ group. In some embodiments, the composition comprises ether lipids having a molar ratio of 18:0 R¹ groups to 18:1 R¹ groups in the range of from 0.3:1 to 1.2:1, from 0.3:1 to 0.9:1, from 0.35:1 to 0.70:1, or about 0.5:1 (e.g. 0.49:1). In some embodiments, ether lipids having an 18:0 R¹ group and ether lipids having an 18:1 R¹ group together comprise at least 50%, at least 75%, at least 80%, at least 85%, at least 90%, at least 95% or at least 98% of the ether lipids in the composition.

In some embodiments, the composition comprises ether lipid molecules having an 18:1 R¹ group, and ether lipid molecules having a 16:0 R¹ group. In some embodiments, the composition comprises ether lipids having a molar ratio of 18:1 R¹ groups to 16:0 R¹ groups in the range of from 1:0.55 to 1:2.3, from 1:1.05 to 1:1.55, from 0.75:1 to 2.9:1, from 1.3:1 to 2:1, or about 1.6:1 (e.g. 1.62:1). In some embodiments, ether lipids having an 18:1 R¹ group and ether lipids having an 16:0 R¹ group together comprise at least 50%, at least 75%, at least 80%, at least 85%, at least 90%, at least 95% or at least 98% of the ether lipids in the composition.

In some embodiments, the composition comprises ether lipid molecules having an 18:0 R¹ group, and ether lipid molecules having a 16:0 R¹ group. In some embodiments, the composition comprises ether lipids having a molar ratio of 18:0 R¹ groups to 16:0 R¹ groups in the range of from 1.4:1 to 1:2.1, from 1:1.05 to 1:2, or about 0.8:1. In some embodiments, ether lipids having an 18:0 R¹ group and ether lipids having an 16:0 R¹ group together comprise at least 50%, at least 75%, at least 80%, at least 85%, at least 90%, at least 95% or at least 98% of the ether lipids in the composition.

In some embodiments, the composition comprises ether lipid molecules having an 18:1 R¹ group, ether lipid molecules having an 18:0 R¹ group, and ether lipid molecules having a 16:0 R¹ group. In some embodiments, the composition comprises ether lipid molecules having an 18:1 R¹ group, ether lipid molecules having an 18:0 R¹ group, and ether lipid molecules having a 16:0 R¹ group, and wherein the molar ratio of 18:0 R¹ groups to 16:0 R¹ groups to 18:1 R¹ groups is in the range of from 0.5:1:3 to 2:1:1, for example about 0.8:1:1.7.

In some embodiments, ether lipids having an 18:1 R¹ group, ether lipids having an 18:0 R¹ group, and ether lipids having an 16:0 R¹ group together comprise at least 50%, at least 75%, at least 80%, at least 85%, at least 90%, at least 95% or at least 98% of the ether lipids in the composition.

In some embodiments, the composition additionally comprises ether lipids having R¹ groups selected from the group consisting of 16:0, 18:2, 20:0 and 20:1.

As discussed above, the ether lipid molecules of Formula (I) have R² and R³ groups.

In some embodiments, the composition comprises ether lipids wherein R² and R³ is hydrogen (e.g. alkylglycerols).

In some embodiments, the composition comprises ether lipids in which R² is hydrogen and R³ is

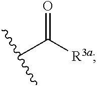

and R^{3a} is selected from the group consisting of a saturated alkyl hydrocarbon, a monounsaturated alkenyl hydrocarbon and a polyunsaturated alkenyl hydrocarbon (e.g. alkyl acyl glycerol).

In some embodiments, the composition comprises ether lipids in which R³ is hydrogen and R² is

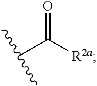

and R^{2a} is selected from the group consisting of a saturated alkyl hydrocarbon, a monounsaturated alkenyl hydrocarbon and a polyunsaturated alkenyl hydrocarbon (e.g. alkyl acyl glycerol).

In some embodiments, the composition comprises ether lipids in which R² is:

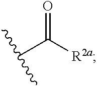

### R³ is

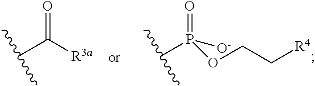

wherein R^{2a} is selected from the group consisting of a saturated alkyl hydrocarbon, a monounsaturated alkenyl hydrocarbon and a polyunsaturated alkenyl hydrocarbon; R^{3a} is selected from the group consisting of a saturated alkyl hydrocarbon, a monounsaturated alkenyl hydrocarbon and a polyunsaturated alkenyl hydrocarbon; and R⁴ is —N(Me)₃⁺ or —NH₃⁺ (e.g. alkyl diacyl glycerol, PC or PE plasmanyl- or plasmenyl-phospholipid).

In embodiments where an alkylglycerol is administered, or where an alkyl acyl glycerol is administered, or where an alkyldiacyl glycerol is administered, references to an alkyl or alkenyl R¹ group having the numbering X:Y means that the group has X carbons, and has Y double bonds.

In embodiments where a plasmalogen is administered, references to an alkyl or alkenyl R¹ group having the numbering X:Y means that the group has X carbons, and has Y double bonds in addition to the vinyl ether.

In some embodiments, the composition comprises additional components in addition to the ether lipid molecules. For example, the composition may contain free fatty acids, such as omega-3 or omega-6 fatty acids.

In some embodiments, the composition is an ether lipid-containing composition according to the Examples.

### Method of Making Compositions

Some aspects of the present disclosure relate to the provision of new compositions containing mixtures of ether lipid molecules of Formula (I). For the avoidance of doubt, the present disclosure relates to new compositions per se, as well as to uses of compositions and methods of using them.

As discussed above, it will be appreciated that the constituents of the formulation may be varied according to the intended purpose of the formulation (e.g. to maintain an in vivo ether lipid profile in a subject versus moving an in vivo ether lipid profile in a subject towards a healthy profile.

The compositions may be prepared by any suitable means. The composition may for example be prepared by mixing a plurality of ether lipids, in ratios and/or levels associated with a non-disease state in vivo. The desired amounts of each component of the composition can be combined and blended to provide a uniform mixture.

Ether lipids, and mixtures of ether lipids, may for example be prepared synthetically. Alkyl glycerols (i.e. compounds of formula (I) wherein R² and R³ are hydrogen), can for example be obtained from commercial sources, and combined to provide a composition having the desired proportions of alkenyl ether and alkyl ether groups. For example, batyl alcohol (an alkyl glycerol having an 18:0 alkyl ether group) is available from Sigma Aldrich and selachyl alcohol (an alkenyl glycerol having an 18:1 alkenyl ether group) is available from Alfa Chemistry. Alkylglycerols (such as batyl alcohol, chimyl alcohol and selachyl alcohol) may be prepared synthetically. Synthesis of these compounds is well known in the art (see, for instance, Takaishi et al., U.S. Pat. No.4,465,869, UK Patent 1,029,610, and Magnusson et al., Tetrahedron (2011) 67, or WO2013/071418, which are hereby incorporated by reference herein in their entirety). In addition, mono- and di-esters of alkylglycerols are well-known in the art and their syntheses have been described (see, e.g., Burgos et al. (1987), J. Org. Chem. 52: 4973-4977; Hirth et al. (1982) Helv. Chim. Acta 65: 1059-1084; and Hirth et al. (1983) Hely. Chim. Acta 66: 1210-1240).

Plasmalogens may be prepared synthetically. Synthesis of these compounds is well known in the art (see, for instance, Shin et al. (2003) J Org. Chem., 2003 68(17): 6760-6766; Van den Bossche, et al. (2007) J. Org. Chem. 72(13): 5005-5007 and Khan et al., International Publication No. WO 2013/071418, which are incorporated herein by reference in their entirety).

Chiral ether lipids may be used in racemic, enantiomerically enriched, or enantiomerically pure forms. For example, some commercially available ether lipids are provided as mixtures of enantiomers. However, ether lipids obtained from natural sources are typically obtained as a single enantiomer. In some embodiments, chiral ether lipids present in the composition are present as a single enantiomer (e.g. the R form, or the S form). In some embodiments, chiral ether lipids present in the composition as a mixture of enantiomers (e.g. in racemic form).

It will be appreciated that, whilst some embodiments of the present disclosure relate to the use of novel compositions, that some known compositions containing a mixture of ether lipids of Formula (I) may also be of use in maintaining or modifying in vivo ether lipid levels and/or ratios. Accordingly, in some embodiments, the present disclosure relates to methods and/or uses utilising existing ether lipid compositions. For example, alkylglycerols may be extracted from a natural source, illustrative examples of which include fish oils such as shark oils, and hematopoietic organs such as bone marrow and spleen. In specific embodiments, alkylglycerols are extracted from fish liver oils, particularly liver oils of elasmobranch fish such as sharks (e.g., Greenland shark, dogfish, ratfish, rabbitfish see, e.g., Hallgren et al., U.S. Pat. No.4,046,914, which is incorporated by reference herein in its entirety), rays, Seamouse etc. Shark liver oil may be obtained commercially (see, e.g., ALKYROL, Eurohealth, Inc., Parkside, Pa.). Common fatty alcohols found in shark liver oil are chimyl alcohol, batyl alcohol and selachyl alcohol. Non-limiting methods for extracting alkylglycerols are disclosed for example in Hallgren et al. (supra) and Brohult et al., International Publication No. WO 1998/52550, which is incorporated by reference herein in its entirety).

Plasmalogens may be prepared from any suitable source. For example, they may be extracted from a natural source, such as but not limited to microorganisms and animals. Non-limiting examples of plasmalogen-producing microorganisms anaerobic bacteria, suitably from the family Acidaminococcaceae, which are intestinal bacteria. Representative examples of plasmalogen-producing animals include birds, mammals, fishes, shellfishes, and the like. In some embodiments, the mammals are livestock mammals, representative examples of which include cow, pig, horse, sheep, goat, and the like. Suitable plasmalogen-containing mammalian tissues include skin, spinal cord, brain, intestines, heart, genitals, and the like. Examples of birds include chicken, domestic duck, quail, duck, pheasant, ostrich, turkey, and the like. There is no particular limitation to an avian tissue to be used. For example, bird meat (in particular, bird's breast meat), bird skin, internal organs of birds, bird eggs etc., are suitably used. Two or more types of different tissues from one or more species of organisms may be used in combination. Methods for extracting plasmalogens are known in the art, non-limiting examples of which are described in Nishimukai etal. (2003) Lipids 38(12): 1227-1235, Herrmann etal., U.S. Pat. No. 4,613,621 and Mawatari et al., U.S. Publication No. 2013/0172293, which are incorporated herein by reference in their entirety.

### Products

The composition may be an emulsion, suspension, or other mixture, and can be combined with one or more other ingredients to form a product.

The product may be a cream, gel, tablet, liquid, pill, capsule, or extruded product. The product may be a food, food ingredient, drink ingredient, nutritional supplement, cosmetic or cosmetic ingredient.

The food may be animal feed, aquaculture feed.

The product may be a food ingredient for e.g. infant formulae, children formula, adult formula, yoghurts, beverages, elderly supplement, ultra-high temperature processed (UHT) drinks (e.g. milk), soup, dips, pasta products, bread, snacks and other bakery products processed cheese, and/or animal feed (including aquaculture feed).

In some embodiments, the composition is in the form of a composition for addition to a food or beverage. In some embodiments, the composition is in the form of a product which is a dietary supplement, capsule, liquid, syrup, food or beverage. For example, a subject may take a capsule containing the composition as a health or nutritional supplement, e.g. on a daily basis. As a further example, the ether lipids may be incorporated into a health food product such as a nutrition bar.

As discussed above, it has been identified that the presence of compounds having certain ether lipids in amount/ratio ranges in the plasma lipidome is associated with improved health profiles in infants. Accordingly, the present disclosure provides compositions for use in infant products such as formula milk containing ether lipid molecules of Formula (I), which can influence the plasma lipidome profile in infants. Thus, in some embodiments, the composition is present in the form of a product which is a liquid infant formula milk, an infant formula milk powder, a supplement for addition to infant formula milk, a supplement for addition to infant food, or an infant dietary supplement.

For example, a mixture of ether lipid molecules of Formula (I) in desired amounts and ratios may be added as a component of an infant formula milk powder, ready for admixing with water to form a liquid infant milk. Alternatively, the ether lipid molecules may be present in a ready-made liquid formula milk product in the desired amounts. As a further example, a supplement containing the ether lipid molecules (e.g. a concentrate) may be provided, from which doses of the ether lipid molecule composition may be taken and added to infant formula milk or, when the infant is old enough to ingest foods, for adding to those foods.

Ether lipid compositions can be incorporated into infant formulae using procedures known in the art. Reference to US2015/0148316 and WO 2015/196250 may be made for suitable formulations.

Infant milk formula is typically a manufactured food intended for infants (children up to 12 months of age). Typical ingredients include purified cow's milk whey and casein (protein source), a blend of vegetable oils as fat source, lactose as a carbohydrate source, and vitamins and minerals. In some cases, soy-based protein formulas can be used. Further variants include infant formulae containing protein hydrolysates.

Most commonly, infant milk formula is provided as a dry powder for reconstitution with sterile water, and the resulting liquid milk is then fed to the child. However, ready-made liquid milk formula products are also available, e.g. in cartons which can be transferred to feeding bottles.

Typically, infant formula milk or infant formula milk powder does not contain human breast milk.

In one embodiment, infant formula milk or infant formula milk powder does not consist of pure non-human animal milk.

In one embodiment, infant formula excludes breast milk and pure milk produced by a non-human animal, although the formula may comprise components derived from milk proteins or carbohydrates.

Products for administration for infants will typically contain a suitable concentration of ether lipid molecules of Formula (I) so as to influence the in vivo plasma lipidome towards a distribution of lipids that is associated with positive health and growth outcomes.

In one non-limiting embodiment, infant formula is supplemented with about 0.05% to about 5% by weight of the composition as described herein.

In some embodiments, the composition comprises ether lipid molecules of Formula (I) in an amount such that, when present in liquid infant formula milk, the concentration of total ether lipid molecules of Formula (I) is in the range of from 50 to 20001 For example, if present in a liquid milk composition, the concentration of total ether lipid molecules is within the stated range. If present, for example, in an infant formula milk powder product, the concentration of ether lipid molecules of Formula (I) present in the powder is sufficient to provide a concentration within the stated range when made up into a liquid milk in accordance with preparation instructions.

In some embodiments, the composition comprises ether lipid molecules of Formula (I) in an amount such that, when present in liquid infant formula milk, the concentration of total ether lipid molecules of Formula (I) is in the range of from 75 to 125, 50 to 180, 90-115, 60-170, 75-140, or 55-19004.

In some embodiments, the composition comprises ether lipid molecules of Formula (I) in an amount such that, when present in liquid infant formula milk, the concentration of total ether lipid molecules of Formula (I) is in the range of from 20 to 400, from 20 to 300, from 20 to 200, from 50 to 400, from 50 to 300, from 50 to 200, from 75 to 400, from 75 to 300, or from 75 to 200 μM.

In some embodiments, the composition comprises ether lipid molecules of Formula (I) in an amount such that, when present in liquid infant formula milk, the concentration of total ether lipid molecules of Formula (I) is in the range of from 75 to 140 μM.

In some embodiments, the composition comprises ether lipid molecules of Formula (I) in an amount such that, when present in liquid infant formula milk, the concentration of total ether lipid molecules of Formula (I) is about 99, about 102 or about 117 μM.

In some embodiments, the composition comprises ether lipid molecules of Formula (I) in an amount such that, when present in liquid infant formula milk, the concentration of total ether lipid molecules of Formula (I) is in the range of from 90 to 120 μM.

In one embodiment, the ether lipid may be encapsulated or entrapped in the food ingredient being more stable when added to a product than unencapsulated or unentrapped.

In one embodiment, the product comprises an omega-3 polyunsaturated fatty acid. In one embodiment, the omega-3 polyunsaturated fatty acid is selected from one or more of: α-linolenic acid (ALA), eicosapentaenoic acid (EPA), and docosahexaenoic acid (DHA) and docosapentaenoic acid (DPA).

In one embodiment, the product comprises the composition in an amount sufficient to maintain or modulate ether lipid levels in the subject or tissue. The optimal amount of the composition may be determined through routine trial and may range between 0.001% to 50% of the product by weight, or 0.05% to about 0.5%, 0.05% to about 5% by weight ether lipid mixtures.

Reference to a “subject” or “individual” or “patient” includes any human (of any age), primate, mammalian, or other species of veterinary or agricultural importance, or test organism known to the skilled person. Reference to a subject or patient indicates that the subject has been diagnosed with a condition such as metabolic disease, diabetes, obesity and its sequelae.

Reference to “maintain” or “maintenance” in relation to ether lipids relates to compositions which, for a period of time, retain the ether lipid molecule levels or ratios for the defined molecules at levels and/or ratios associated with a non-disease state and within plus or minus about 2 SD (standard deviations) in a population. Suitable populations are illustrated in Example 1. In one embodiment, the ether lipids are for maintenance of plasmanyl- and/or plasmenyl-phospholipid levels and/or ratios.

Reference to “modulate” or “modify” or the like in relation to ether lipid molecules refers to compositions which, for a period of time, change the ether lipid levels for the defined molecules towards levels and/or ratios associated with a non-disease state and within plus or minus about 2 SD (standard deviations) in a population. Suitable populations are illustrated in Example 1. In one embodiment, the ether lipids are for modifying plasmanyl- and/or plasmenyl-phospholipid levels and/or ratios. Modulation, may be down modulating or up modulating or down modulating and up modulating particular ether lipid molecules as described herein.

In one embodiment, modifying of one or more lipid species includes administration of a defined mixture of ether lipid molecules, such as plasmanyl- and/or plasmenyl-phospholipid to down modulate the proportion of ether lipid molecules identified herein as risk factors for metabolic disease such as diabetes. In one embodiment, modifying of one or more lipid species includes administration of a defined mixture of ether lipid molecules, such as plasmanyl- and/or plasmenyl-phospholipid to up modulate the proportion of ether lipid molecules identified herein as protective factors for metabolic disease such as diabetes. For example, chains 16:0 and 20:0 are identified as risk factors in Example 1. In one embodiment, modulations is toward in vivo levels or ratios of ether lipid molecules identified herein as associated with a non-disease state.

Reference to “reference ether lipid molecule or side chain profile” includes a profile of ether lipid molecules established from a control population, such as a non-disease population or a disease population, or from a particular subject including the subject at an earlier time point.

Reference to “healthy or non-disease levels or ratios of ether lipid molecules” includes particular molar ratios or proportions or % by weight of two or more ether lipid species determined herein to be associated with a population of healthy humans.

### Administration of Compositions

Compositions comprising mixtures of ether lipid molecules as described herein are administered in an effective amount sufficient to maintain or promote in the subject or tissue a non-disease ether lipid molecule profile or to modulate levels or ratios of defined ether lipid molecule in the subject towards a non-disease state as described herein. Products comprising the herein defined compositions are also contemplated.

Accordingly, provided herein is a method of maintaining ether lipids in a subject at levels and/or ratios associated with a non-disease state, or of modifying ether lipids in a subject towards levels and/or ratios associated with a non-disease state, comprising administering an effective amount of a composition as defined herein to the subject.

In some embodiments, the method is for maintenance or modification of plasmanyl- and/or plasmenyl-phospholipid levels and/or ratios in a subject.

The compositions and products described herein find use in maintaining a healthy plasma lipidome profile or modifying a plasma lipidome profile towards a more healthy profile, and in reducing the likelihood of a subject developing conditions such as dyslipidemia or metabolic disease. Accordingly, there is also provided a method of treating or preventing conditions which are associated with an unhealthy plasma lipidome profile, such as metabolic disease, diabetes, cardiovascular disease, obesity, overweight, fatty liver disease, an inflammatory condition or dyslipidemia in a subject, the method comprising administering an effective amount of a composition or product as described herein to the subject.

There is also provided herein a composition or product as described herein for use in therapy, for example for use in treating or preventing metabolic disease, diabetes, cardiovascular disease, obesity, overweight, fatty liver disease, an inflammatory condition or dyslipidemia in a subject.

There is also provided herein use of a composition or product as described herein for the manufacture of a medicament for the prevention or treatment of metabolic disease, diabetes, cardiovascular disease, obesity, overweight, fatty liver disease, an inflammatory condition or dyslipidemia in a subject.

As discussed above, it has been identified that breast milk has a different ether lipid profile to animal milks or formula milks, that the nature of infant diet is associated with a different plasma lipidome profile, and that the nature of the infant plasma lipidome profile is associated with health and growth outcomes, e.g. in relation to risk of being overweight, obese or asthmatic or other inflammatory conditions.

Accordingly, there is provided a method of preventing asthma, an inflammatory condition, obesity or overweight in an infant subject, the method comprising administering an effective amount of a composition or product as defined herein, particularly a composition which in the form of an infant product as discussed above, to the infant subject.

There is also provided a composition or product as described herein, particularly a composition in the form of an infant product as discussed above, for use in preventing obesity, overweight, asthma or an inflammatory condition in an infant subject.

There is also provided use of a composition or product as described herein, for the manufacture of a medicament for the prevention of asthma, an inflammatory condition, obesity or overweight in a subject.

Any suitable administration regime may be followed. Administration of the composition or product may be on a daily, twice to about 10x daily, weekly, bi-weekly, three weekly, monthly or ad hoc basis depending upon the subject, and for example the formulation employed.

In the case of supplementation of infant formula, the composition may be provided as a component of infant formula milk and administered for example as part of the normal daily diet.

The production of the maintenance or modulatory compositions may for example comprise mixing the two or more ether lipid as described herein with a pharmaceutically or physiologically acceptable carrier.

The terms “effective amount” including “therapeutically effective amount” and “prophylactically effective amount” or “physiologically effective amount” as used herein mean a sufficient amount of a composition of the present application either in a single dose or as part of a series or slow release system which provides the desired therapeutic, preventative, or physiological effect in some subjects. Undesirable effects, e.g. side effects, may sometimes manifest along with the desired therapeutic effect; hence, a practitioner balances the potential benefits against the potential risks in determining an appropriate “effective amount”. The exact amount of composition required will vary from subject to subject, depending on the species, age and general condition of the subject, mode of administration and the like. Thus, it may not be possible to specify an exact ‘effective amount’. However, an appropriate ‘effective amount’ in any individual case may be determined by one of ordinary skill in the art using routine skills or experimentation. One of ordinary skill in the art would be able to determine the required amounts based on such factors as prior administration of the compositions or other agents, the subject's size, the severity of a subject's symptoms or the severity of symptoms in a population, and the particular composition or route of administration selected.

The term “treating” or “treatment”, for example in relation to metabolic disease, such as obesity or diabetes, or dyslipidemia refers to any measurable or statistically significant amelioration of metabolic disease, such as diabetes, obesity, or dyslipidemia. The term “treating” or “treatment” in relation modulating the in vivo defined ether lipid composition towards a non-disease profile means modulating the in vivo defined ether lipid composition towards a non-disease profile. This can be assessed by measuring the herein defined ether lipid profile of the subject before and after administration. As used herein, the use of the terms “treating” and “treatment” in relation to a condition, disease or disorder, may include reducing the severity of the condition, disease or disorder, or reducing the severity and/or frequency of one or more symptoms of the condition, disease or disorder.

The terms “prevention” or “prophylaxis” relates to maintaining the in vivo defined ether lipid profile at or substantially the same as the non-disease profile identified herein. This can be assessed by periodically measuring the herein defined ether lipid profile of the subject. As used herein, the use of the terms “prevention” and “preventing” in relation to a condition, disease or disorder, may include reducing the likelihood that a subject will develop such a condition disease or disorder.

The present application provides methods of maintaining an in vivo defined ether lipid profile at or substantially the same as a reference non-disease profile identified herein by periodic supplementation of the composition or products as defined herein.

A “pharmacologically acceptable” composition is one tolerated by a recipient subject. It is contemplated that an effective amount of the composition is administered. An “effective amount” is an amount sufficient to achieve a desired biological effect such as to maintain or modulate an ether lipid molecule profile in the subject for a period of time. Monitoring may by any convenient method known in the art. The actual effective amount say be dependent upon the type of subject/species their age, sex, health, and weight. Examples of desired biological effects include maintaining or modulating two or more ether lipid or plasmalogen species towards their healthy level as determined herein, or reducing the level of one or more ether lipid or plasmalogen species determined herein to be risk factors for metabolic disease, diabetes, and their sequelae. In some embodiments, physiologically significant changes may only be achieved after a course of treatment in a proportion of suitable subjects.

The compositions of the present application can be administered as the sole active pharmaceutical agent, or used in combination with one or more agents to maintain or beneficially modulate ether lipid molecule profiles in a subject. Profiles are readily determined using the protocols described herein.

The present disclosure also encompasses compositions, particularly pharmaceutical compositions, comprising the composition as defined herein together with a pharmaceutically acceptable carrier and/or diluent.

A pharmaceutical composition may comprise the ether lipid mixture as described herein, in combination with a standard, well-known, non-toxic pharmaceutically-acceptable carrier, adjuvant or vehicle such as phosphate-buffered saline, water, ethanol, polyols, vegetable oils, a wetting agent or an emulsion such as a water/oil emulsion. The composition may be in either a liquid or solid form. For example, the composition may be in the form of a tablet, capsule, ingestible liquid, spray, or powder, injectable, or topical ointment or cream. Proper fluidity can be maintained, for example, by the maintenance of the required particle size in the case of dispersions and by the use of surfactants. It may also be desirable to include isotonic agents, for example, sugars, sodium chloride, and the like. Besides such inert diluents, the composition can also include adjuvants, such as wetting agents, emulsifying and suspending agents, sweetening agents, flavouring agents and perfuming agents.

Suspensions, in addition to the active compounds, may comprise suspending agents such as ethoxylated isostearyl alcohols, polyoxyethylene sorbitol and sorbitan esters, microcrystalline cellulose, aluminum metahydroxide, bentonite, agar-agar, and tragacanth or mixtures of these substances.

Solid dosage forms such as tablets and capsules can be prepared using techniques well known in the art. For example, ether lipid mixtures produced in accordance with the present disclosure can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant fatty acid(s).

For intravenous administration, the composition may be incorporated into commercial formulations. Examples of pharmaceutically acceptable carriers and methods of manufacture of multiple composition formats may be found in the most recent edition of Remington's Pharmaceutical Sciences, Mack Publishing, Easton.

A typical dosage of a composition as described herein is from 0.1 mg to 20 g, taken from one to five times per day and is preferably in the range of from about 10 mg to about 1, 2, 5, or 10 g daily (taken in one or multiple doses). Non-limiting illustrative doses of a composition as described in the present application are 100 to 3000 mg once or twice daily. In another example the composition is added to an oral product and administered at a percent by weight of 0.01% to 10% of the product.

Possible routes of administration of the pharmaceutical compositions of the presently described compositions include, for example, enteral (e.g., oral and rectal) and parenteral. For example, a liquid preparation may be administered orally or rectally. Additionally, a homogenous mixture can be completely dispersed in water, admixed under sterile conditions with physiologically acceptable diluents, preservatives, buffers or propellants to form a spray or inhalant.

The dosage of the composition to be administered to the subject may be determined by one of ordinary skill in the art and depends upon various factors such as weight of the subject, age and species of the subject, overall health of the subject, past history of the subject, immune status of the patient, etc.

Additionally, the compositions of the present disclosure may be utilized for cosmetic purposes. It may be added to pre-existing cosmetic compositions such that a mixture is formed and may be used as the sole “active” ingredient in a cosmetic composition.

### Risk Assessments

As described in the present application the subject compositions may be used preventatively for example, in the case of diabetes by reducing the proportions of alkenyl or acyl ether lipid side chains that have been identified as risk factors for the development of diabetes (incident diabetes) or markers for diabetes (prevalent diabetes).

Thus, for example, increasing alkenyl species O-16:0 and O-20:0 and acyl species 18:1 and 20:3 are identified in subject with diabetes and not in control subjects and are targeted for reduction.

In one embodiment, alkenyl species 15:0, 17:0, 18:0 and 19:0 are identified as protective factors and one or more are targeted for an increase.

In one embodiment, alkenyl O-16:0 and acyl 20:4 are identified as a risk factor for the development of diabetes in subjects not displaying symptoms of diabetes or pre-diabetes and one or more are targeted for reduction. In one embodiment, acyl species 18:2 are identified as protective factors that reduce the risk of developing pre-diabetes or diabetes, and are targeted for an increase as a method of prophylaxis.

In one embodiment, the ether lipids are PE(P). In another embodiment the ether lipids are one or more of PE(P), PC(P), PC(O), PE(O), LPC(O) which are under similar regulation as identified herein.

Accordingly, in one embodiment, the present application extends to monitoring for levels in the above identified ether lipids with a view to providing prophylactic or therapeutic ether lipid compositions as described herein.

Illustrative methods capable of analysing lipid species include classical lipid extraction methods, mass spectrometry together with electrospray ionization and matrix-assisted laser desorption ionisation, with mass analysis such as quadruple and/or TOF (e.g. Quadrapole/TOF) or orbitrap mass analysers. Chromatographic methods are used for the separation of lipid mixtures such as gas chromatography, high pressure liquid chromatography (HPLC), ultra-high pressure liquid chromatography (UHPLC), capillary electrophoresis (CE). These may be used with mass spectrometry based detection systems or other detectors including optical detectors. Clinical mass spectrometry systems are used by clinical laboratories to provide lipid profiles and ratios upon request. Another suitable technique for quantitative lipid analysis is one or two dimensional nuclear magnetic resonance (NMR). Two dimensional techniques such as heteronuclear single quantum coherence (HSQC) are suitable for lipid profiling through the ability to elucidate C-H bonds within a structure. Any technique capable of identifying individual lipid species in the sample can be used for collecting information on the lipid species. Typically, MS is used coupled to a separation method such as various forms of chromatography.

In one embodiment enzymatic methods known in the art may be used to identify lipid classes or subclasses and/or species.

Lipid level data may be processed to produce a report of levels and/or ratios. In one embodiment, lipid data are processed as described herein to identify and/or report the risk that a subject will develop pre-diabetes or diabetes or to monitor treatment protocols.

The methods enabled herein permit integration into pathology architecture or platform systems.

For example, the method described herein allows a user or client to determine metabolic disease including pre-diabetes or diabetes risk status, or treatment response profile of an individual, the method including: (a) receiving data in the form of lipid levels, relative lipid levels or signature profiles developed from an individual's tissue, plasma or blood sample from the user via a communications network; (b) processing the individuals data via an algorithm which provides one or more status/risk value/s by comparing levels and/or ratios of lipid levels to those from one or more reference levels or ratios.

In some embodiments, an indication of the risk/status transmitted to the user is transferred via a communications network. It will also be appreciated that in one example, the end stations can be hand-held devices, such as PDAs, mobile phones, or the like, which are capable of transferring the subject data to the base station via a communications network such as the Internet, and receiving the reports. When a server is used, it is generally a client server or more particularly a simple object application protocol (SOAP).

In one embodiment, the method is suitable to be practised as a home test kit or point-of-care method typically employing a device suitable for home use or point of care.

The kits or panels can be used in a laboratory or in a home use test kit. Blood, for example may be dried down onto a support material suitable for analysis at home or sent to a laboratory for analysis.

Biosensor technologies that permit less expensive equipment or fewer trained personnel are available for developing devices for lipid species analysis that may be used at point of care. This is particularly useful when as here a small number of lipid species can provide prognostic or monitoring data. Biosensors which recognise a target molecule and produce a measurable or observable signal may be for example, optical, electrochemical or mechanical biosensors. Assays that use a label indirectly measure the binding of an analyte lipid to a target molecule using a reporter molecule as an indication of binding and amount. Label free assays measure signal changes directly associated with target binding or cellular processes. Examples of label free optical sensors include surface plasmon resonance sensing (SPR), Interferometry (such as backscattering inferometry (BSI), ellipsometry, and assays based on UV absorption of lipid-functionalized gold nanorods. In optical assays using labels, the target lipid molecule is immobilized on the surface of a biosensor and then probed with a binding agent, such as an antibody couples to a label (many labels are known to the skilled person such as a fluorophore, quantum dot, radioisotope, enzyme). Reference may be made to Sakamuri et al. “Detection of stealthy small amphiphilic biomarkers Journal of Microbiological Methods 103: 112-117, 2014. These authors have used a waveguide based biosensor measuring only surface attached fluorescence antibody signals to detect lipids and amphiphilic targets in biological samples. Electrochemical sensors use an electrode to directly detect a reaction, typically a current from electron transfer during binding of an analyte and a chemically functionalized surface. Potentiometric sensors usefully measure charge accumulation to detect lipid antigens such as amphiphilic cholesterol using lipid films. Mechanical sensors are ideal for clinical applications and include cantilever and quartz crystal microbalances (QCM). The later detects changes in resonance frequency on the sensor surface from increased mass due to analyte binding.

In one embodiment the method is an enzyme-linked immunosorbent (ELISA)-type, flow cytometry, bead array, lateral flow, cartridge, microfluidic or immunochromatographic or enzyme-substrate based method or the like.

Typically, such methods employ binding agents such as an antibody or an antigen-binding fragment thereof. Other suitable binding agents are known in the art and include antigen binding constructs such as affimers, aptamers, or suitable ligands (receptors) or parts thereof

Antibodies, such as monoclonal antibodies, or derivatives or analogues thereof, include without limitation: Fv fragments; single chain Fv (scFv) fragments; Fab′ fragments; F(ab′)2 fragments; humanized antibodies and antibody fragments; camelized antibodies and antibody fragments, and multivalent versions of the foregoing. Multivalent binding reagents also may be used, as appropriate, including without limitation: monospecific or bispecific antibodies; such as disulfide stabilized Fv fragments, scFv tandems (scFv) fragments, diabodies, tribodies or tetrabodies, which typically are covalently linked or otherwise stabilized (i.e. leucine zipper or helix stabilized) scFv fragments.

Methods of making antigen-specific binding agents, including antibodies and their derivatives and analogues and aptamers, are well-known in the art. Polyclonal antibodies can be generated by immunization of an animal. Monoclonal antibodies can be prepared according to standard (hybridoma) methodology. Antibody derivatives and analogues, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163; 5,475,096; 5,840,867 and 6,544,776.

The present disclosure also encompasses the following aspects and embodiments set out in the clauses below:
1. A composition comprising a mixture of ether lipid molecules of Formula (I):

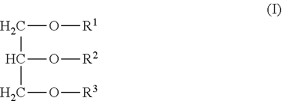

wherein

R¹ is an alkyl or alkenyl group;

R² is hydrogen or

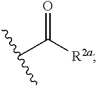

and

R³ is hydrogen,

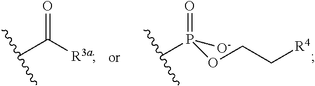

wherein

R^{2a} and R^{3a} are each an alkyl or alkenyl group;

R⁴ is —N(Me)₃⁺ or —NH₃⁺; and
wherein the composition is for in vivo maintenance of ether lipids at levels and/or ratios associated with a non-disease state, or wherein the composition is for in vivo modification of ether lipids towards levels and/or ratios associated with a non-disease state.
2. The composition according to clause 1, wherein the composition is for in vivo maintenance or in vivo modification of plasmanyl- and/or plasmenyl-phospholipid levels and/or ratios.
3. The composition according to clause 1 or clause 2, wherein the composition comprises ether lipid molecules having an 18:0 alkyl R^{1a} group, and ether lipid molecules having an 18:1 alkenyl R¹ group.
4. The composition according to any one of clauses 1 to 3, wherein the composition is for in vivo maintenance of ether lipids at or in vivo modification of ether lipids towards an in vivo total ether lipid profile in which the ether lipids have a molar ratio of 18:0 alkyl ether groups to 18:1 alkenyl ether groups of from 1.2:1 to 2.5:1.
5. The composition according to any one of clauses 1 to 4, wherein the composition is for in vivo maintenance of ether lipids at or in vivo modification of ether lipids towards an in vivo total ether lipid profile in which the ether lipids have a molar percent of 18:0 alkyl ether groups in the range of from 32.6% to 45.8%, and a molar percent of 18:1 alkenyl ether groups in the range of from 18.6% to 27.9%.
6. The composition according to any one of clauses 1 to 5, wherein the composition comprises ether lipids having a molar ratio of 18:0 alkyl R¹ groups to 18:1 alkenyl R¹ groups in the range of from 1.2:1 to 2.5:1.
7. The composition according to any one of clauses 1 to 6, wherein the composition comprises ether lipid molecules having an 18:1 alkenyl R¹ group, and ether lipid molecules having a 16:0 alkyl R¹ group.
8. The composition according to any one of clauses 1 to 7, wherein the composition is for in vivo maintenance of ether lipids at or in vivo modification of ether lipids towards an in vivo total ether lipid profile in which the ether lipids have a molar ratio of 18:1 alkenyl ether groups to 16:0 alkyl ether groups in the range of from 0.5:1 to 1:1.
9. The composition according to any one of clauses 1 to 8, wherein the composition is for in vivo maintenance of ether lipids at or in vivo modification of ether lipids towards an in vivo total ether lipid profile in which the ether lipids have a molar percent of 18:1 alkenyl ether groups in the range of from 18.6% to 27.9%, and a molar percent of 16:0 alkenyl ether groups in the range of from 26.8% to 37.4%.
10. The composition according to any one of clauses 1 to 9, wherein the composition comprises ether lipids having a molar ratio of 18:1 alkenyl R¹ groups to 16:0 alkyl R¹ groups in the range of from 0.5:1 to 1:1.
11. The composition according to any one of clauses 1 to 10, wherein the composition comprises ether lipid molecules having an 18:0 alkyl R¹ group, and ether lipid molecules having a 16:0 alkyl R¹ group.
12. The composition according to any one of clauses 1 to 11, wherein the composition is for in vivo maintenance of ether lipids at or in vivo modification of ether lipids towards an in vivo total ether lipid profile in which the ether lipids have a molar ratio of 18:0 alkyl ether groups to 16:0 alkyl ether groups in the range of from 0.9:1 to 1.7:1.
13. The composition according to any one of clauses 1 to 12, wherein the composition is for in vivo maintenance of ether lipids at or in vivo modification of ether lipids towards an in vivo total ether lipid profile in which the ether lipids have a molar percent of 18:0 alkyl ether groups in the range of from 32.6% to 45.8%, and a molar percent of 16:0 alkyl ether groups in the range of from 26.8% to 37.4%.
14. The composition according to any one of clauses 1 to 13, wherein the composition comprises ether lipids having a molar ratio of 18:0 alkyl R¹ groups to 16:0 alkyl R¹ groups in the range of from 0.9:1 to 1.7:1.
15. The composition according to any one of clauses 1 to 14, wherein the composition comprises ether lipid molecules having an 18:1 alkenyl R¹ group, ether lipid molecules having a 18:0 alkyl R¹ group, and ether lipid molecules having a 16:0 alkyl R¹ group.
16. The composition according to any one of clauses 1 to 15, wherein the composition is for in vivo maintenance of ether lipids at or in vivo modification of ether lipids towards an in vivo total ether lipid profile in which the ether lipids have a molar ratio of 18:1 alkenyl ether groups to 18:0 alkyl ether groups to 16:0 alkyl ether groups of about 1:1.7:1.4.
17. The composition according to any one of clauses 1 to 16, wherein the composition is for in vivo maintenance of ether lipids at or in vivo modification of ether lipids towards an in vivo total ether lipid profile in which the ether lipids have a molar percent of 18:1 alkenyl ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%.
18. The composition according to any one of clauses 1 to 17, wherein the composition comprises ether lipids having a molar ratio of 18:1 alkenyl R¹ groups to 18:0 alkyl R¹ groups to 16:0 alkyl R¹ groups of about 1:1.7:1.4.
19. The composition according to clause 19, wherein ether lipids having an 18:1 alkenyl R¹ group, ether lipids having an 18:0 alkyl R¹ group, and ether lipids having a 16:0 alkyl R¹ group together comprise at least 50% of the ether lipids in the composition.
20. The composition according to any one of clauses 1 to 19, wherein the composition additionally comprises ether lipids having R¹ groups selected from the group consisting of 15:0 alkyl, 17:0 alkyl, 19:0 alkyl, 20:0 alkyl, and 20:1 alkenyl.
21. The composition according to any one of clauses 1 to 20, wherein the composition comprises ether lipids wherein R² and R³ is hydrogen.
22. The composition according to any one of clauses 1 to 20, wherein the composition comprises ether lipids in which R² is hydrogen and R³ is

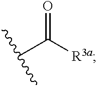

and
R^{2a} is selected from the group consisting of a saturated alkyl hydrocarbon, a monounsaturated alkenyl hydrocarbon and a polyunsaturated alkenyl hydrocarbon.
23. The composition according to any one of clauses 1 to 22, wherein the composition comprises ether lipids in which R³ is hydrogen and R² is

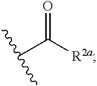

and
R^{2a} is selected from the group consisting of a saturated alkyl hydrocarbon, a monounsaturated alkenyl hydrocarbon and a polyunsaturated alkenyl hydrocarbon.
24. The composition according to any one of clauses 1 to 23, wherein the composition comprises ether lipids in which
   R² is:

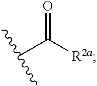

R³ is

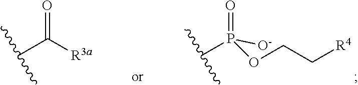

wherein
R^{2a} is selected from the group consisting of a saturated alkyl hydrocarbon, a monounsaturated alkenyl hydrocarbon and a polyunsaturated alkenyl hydrocarbon;
R^{3a} is selected from the group consisting of a saturated alkyl hydrocarbon, a monounsaturated alkenyl hydrocarbon and a polyunsaturated alkenyl hydrocarbon; and
R⁴ is —N(Me)₃⁺ or NH₃⁺.
25. The composition according to any one of clauses 1 to 20 and 22 to 24, wherein the composition comprises ether lipid molecules having a 20:4 acyl alkenyl R² and/or R³ group, ether lipids having a 22:6 acyl alkenyl R² and/or R³ group, and ether lipids having an 18:2 acyl alkenyl R² and/or R³ group.
26. The composition according to any one of clauses 1 to 20 and 22 to 25, wherein the composition is for in vivo maintenance of ether lipids at or in vivo modification of ether lipids towards an in vivo total ether lipid profile in which the ether lipids have a molar ratio of 20:4 acyl alkenyl groups to 22:6 acyl alkenyl groups to 18:2 acyl alkenyl groups of about 3:1.2:1.
27. The composition according to any of clauses 1 to 20 and 22 to 26, wherein the composition is for in vivo maintenance of ether lipids at or in vivo modification of ether lipids towards an in vivo total ether lipid profile in which the ether lipids have acyl alkenyl groups in which the molar percent of 20:4 acyl alkenyl groups is in the range of from 31.3% to 52.5%, the molar percent of 22:6 acyl alkenyl groups is in the range of from 9.3% to 23.9%, and the molar percent of 18:2 acyl alkenyl groups is in the range of from 7.6% to 19.9%.
28. The composition according to any one of clauses 1 to 20 and 22 to 27, wherein the composition comprises ether lipids having a molar ratio of 20:4 acyl alkenyl groups to 22:6 acyl alkenyl groups to 18:2 acyl alkenyl groups of about 3:1.2:1.
29. The composition according to any one of claims 1 to 28, wherein the composition comprises free fatty acids.
30. The composition according to any one of clauses 1 to 29, wherein the composition comprises omega-3 or omega-6 fatty acids.
31. The composition according to any one of clauses 1 to 30, wherein the composition is an ether lipid-containing composition according to the Examples.
32. The composition according to any one of clauses 1 to 31, wherein the composition is prepared by mixing a plurality of ether lipids, in ratios and/or levels corresponding with ratios and/or levels associated with a non-disease state in vivo.
33. A method of assessing a subject for or with a metabolic disease or dyslipidemia in a tissue or a risk of developing same, the method comprising measuring the relative abundance of one or more ether lipid side chains in a biological sample from a subject to obtain a subject ether lipid side chain profile, and (ii) determining the similarity or difference between the ether lipid side chain profile obtained in (i) and a reference ether lipid side chain profile.
34. A method of treating or preventing metabolic disease or dyslipidemia in a subject, the method comprising (i) determining the relative abundance of one or more ether lipid side chains in a biological sample from a subject to obtain a subject ether lipid side chain profile, and (ii) administering a composition of any one of clauses 1 to 32 contingent upon the similarity or difference between the ether lipid side chain profile obtained in (i) and a reference ether lipid side chain profile.
35. The method of clause 33 or 34, wherein the reference ether lipid side chain profile is the profile characteristic of a healthy individual and comprises: ether lipids having a molar ratio of 18:1 alkenyl ether to 18:0 alkyl ether to 16:0 alkyl ether groups of about 1:1.7:1.4;
   and/or
   ether lipids having a molar percent of 18:1 alkenyl ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%.
36. A method of treating or preventing metabolic disease or dyslipidemia in a subject, the method comprising administering an effective amount of a composition of any one of clauses 1 to 32 to the subject.

Illustrative methods and materials used in the Examples are described as follows.

### Lipidomic Analysis

Lipid extraction: Lipidomic analysis was/is performed as described in Huynh et al 2019. Briefly, lipids were/are extracted from milk, plasma (10 μL) or tissue homogenates (50 μg protein equivalents in 10 μL) as described previously (Alshehry Z H, Mundra P A, Barlow C K, Mellett N A, Wong G, McConville M J, et al. Plasma Lipidomic Profiles Improve on Traditional Risk Factors for the Prediction of Cardiovascular Events in Type 2 Diabetes Mellitus. Circulation. 2016; 134(21):1637-50, Weir J M, Wong G, Barlow C K, Greeve M A, Kowalczyk A, Almasy L, et al. Plasma lipid profiling in a large population-based cohort. J Lipid Res. 2013;54(10):2898-908., Rasmiena A A, Barlow C K, Stefanovic N, Huynh K, Tan R, Sharma A, et al. Plasmalogen modulation attenuates atherosclerosis in ApoE- and ApoE/GPx1-deficient mice. Atherosclerosis. 2015; 243(2):598-608.).

For plasma or milk, 10 μL was/is mixed with 100 μL of butanol:methanol (1:1) with 10 mM ammonium formate which contained a mixture of internal standards. Samples were/are vortexed thoroughly and set in a sonicator bath for 1 hour maintained at room temperature. Samples were/are then centrifuged (16,000×g, 10 min, 20° C.) before transferring the into sample vials with glass inserts for analysis.

For tissue or milk analysis, 10 μL of tissue homogenate was/is combined with 200 μL CHCl₃/MeOH (2:1) and 15 μL of internal standard mix then briefly vortexed. Samples were mixed (rotary mixer, 10 min), sonicated (water bath, 30 min) then allowed to stand (20 min) at room temperature. Samples were/are centrifuged (16,000×g, 10 min, 20° C.) and the supernatant was dried under a stream of nitrogen at 40° C. The extracted lipids were/are resuspended in 50 μL, H₂O saturated BuOH with sonication (10 min), followed by 50 μL of 10 mM NH₄COOH in MeOH. Extracts were/are centrifuged (3,350×g, 5 min) and the supernatant transferred into 0.2 mL glass vials with teflon insert caps.

### Mass Spectrometry:

Analysis of extracts was/is performed on an Agilent 6490 QQQ mass spectrometer with an Agilent 1290 series HPLC system and a ZORBAX eclipse plus C18 column (2.1×100 mm 1.8 μm, Agilent) with the thermostat set at 60° C. Mass spectrometry analysis was/is performed in positive ion mode with dynamic scheduled multiple reaction monitoring (MRM). Mass spectrometry settings and MRM transitions for each lipid class, subclass and individual species are shown in Huynh K, Barlow C K, Jayawardana K S, Weir J M, Mellett N A, Cinel M, et al. High-throughput plasma lipidomics: Detailed mapping of the associations with cardiometabolic risk factors. Cell Chemical Biology. 17:26(1), 71-84, 2019.

The solvent system consists of solvent A) 50% H₂O/30% acetonitrile/20% isopropanol (v/v/v) containing 10 mM ammonium formate and solvent B) 1% H₂O/9% acetonitrile/90% isopropanol (v/v/v) containing 10 mM ammonium formate. A stepped linear gradient with a 15-minute cycle time per sample and a 1 μL sample injection is utilised.

The gradient was/is as follows; starting with a flow rate of 0.4 ml/minute at 10% B and increasing to 45% B over 2.7 minutes, then to 53% over 0.1 minutes, to 65% over 6.2 minutes, to 89% over 0.1 minute, to 92% over 1.9 minutes and finally to 100% over 0.1 minute. The solvent was/is then held at 100% B for 0.8 minutes (total 11.9 minutes). Equilibration was/is as follows: solvent was decreased from 100% B to 10% B over 0.1 minute and held for an additional 0.9 minutes. Flow rate was/is then switched to 0.6 ml/minute for 1 minute before returning to 0.4 ml/minute over 0.1 minutes. Solvent B was/is held at 10% B for a further 0.9 minutes at 0.4m1/minutes for a total cycle time of 15 minutes.

The following mass spectrometer conditions were/are used: gas temperature, 150° C., gas flow rate 17 L/min, nebulizer 20 psi, Sheath gas temperature 200° C., capillary voltage 3500V and sheath gas flow 10 L/min. Isolation widths for Q1 and Q3 were set to “unit” resolution (0.7 amu).

PQC samples consisting of a pooled set of 6 healthy individuals were/are incorporated into the analysis at a rate of 1 PQC per 18 samples. TQC consisted of PQC extracts which had been/are pooled and split into individual vials to provide a measure of technical variation from the mass spectrometer only. These were/are included at a rate of 1 TQC per 18 samples. TQCs were monitored for changes in peak area, width and retention time to determine the performance of the LC-MS/MS analysis and were subsequently used to align for differential responses across the analytical batches.

Quantification of lipid species was/are determined by comparison to the relevant internal standard. As previously described (Weir J M, et al. J Lipid Res. 2013; 54(10):2898-908) response factors were/are generated for each cholesteryl ester species to better approximate their true concentrations. Response factors generated are provided in (Huynh K, supra). Similarly, species with non-class specific internal standards had/have response factors generated as previously described (Huynh K, supra).

### Data Analysis

### PE(P) Plasmalogen Sidechain Compositions

PE(P)s are phosphoglycerol lipids with a phosphatidylethanolamine head group and two side-chains on the glycerol backbone: an alkenyl chain and an acyl chain. There are many possible alkenyl and acyl side chains, and most if not all combinations are possible, though only a subset of these are measured (including the most abundant) in our lipidomics methods. The relative abundances (or proportions) of alkenyl and acyl sidechains can be obtained by summing the concentrations of PE(P) lipids sharing the same alkenyl or acyl chains and dividing by the total concentration of all PE(P) species combined. Taken together, the relative abundances of k entities make up a k-part composition. Of note, a composition including the relative abundances of p<k entities of an original k-part composition is a p-part subcomposition.

Association analyses between disease and relative abundances is carried out in the same way as the more traditional associations with individual lipid concentrations.

### Ternary Diagrams

Ternary diagrams are graphical representations wherein each sample can be represented as a point plotted inside a triangle that encompasses all possible 3-part compositions. Each summit of the triangle corresponds to extreme values (i.e. 100%) of one of the 3 parts; each edge corresponds to the opposite extreme (i.e. 0%) of the facing summit. The composition of each of the 3 parts can be read as the distance of the data point between each part's 100% summit or 0% edge.

The present description is further illustrated by the following examples, which should not be construed as limiting in any way.

### EXAMPLE 1

### Lipidomic Analysis of the Australian Diabetes, Obesity and Lifestyle Study

Lipidomic analysis of the Australian Diabetes, Obesity and Lifestyle study (AusDiab) was performed. The AusDiab study was designed to examine the prevalence and risk factors of type 2 diabetes and cardio-vascular disease (CVD) in the Australian population. The AusDiab study cohort analysed here consisted of 4403 male and 5525 female participants who were either normoglycemic (healthy), had prediabetes, or had T2D at the study baseline. This analysis was designed to identify lipid species associated with prevalent diabetes. The same lipidomic profiles were analysed together with longitudinal outcome data in a subcohort. This longitudinal subcohort containing participants who did not have diabetes at baseline or at the five year follow up time point (n=5510) or participants who did not have diabetes at baseline but developed diabetes during the five year follow up period (218 participants).

### The AusDiab Cohort (Prevalent Disease)

Lipidomic analysis was performed on baseline plasma samples from 9,928 participants in the AusDiab study from the 11,247 participants originally recruited. This represented all available plasma samples for which baseline diabetes status measurements were available. This analysis was designed to identify associations between lipid species, risk factors and prevalent clinical endpoints.

The following definitions have been used: normoglycemia-fasting blood glucose (FBG)≤6.0 mmol/L, 2 h post load glucose (2h-PLG)≤7.8 mmol/L; prediabetes-FBG between 6.1 and 6.9 mmol/L, 2h-PLG between 7.8 and 11 mmol/L; diabetes- FBG>6.9 mmol/L or 2h-PLG>11 mmol/L. Characteristics of the cohort are shown in Table 1.

TABLE 1
Demographic and clinical description of the AusDiab cohort.

| Demographic/Clinical | | | | Prevalent | | Prevalent | |
|---|---|---|---|---|---|---|---|
| Variables¹ | | Healthy | | Prediabetes | | Diabetes | |
| N= | | 7818 | | 1424 | | 686 | |
| Sex | (% male) | 43% | | 48% | | 50% | |
| Age | (years) | 49 | (13.7) | 57.5 | (13.7) | 62.3 | (12.5) |
| BMI | (kg/m²) | 26.3 | (4.55) | 28.6 | (5.06) | 30 | (6.13) |
| Total | (mmol/L) | 5.6 | (1.06) | 5.91 | (1.09) | 5.7 | (1.09) |
| cholesterol | | | | | | | |
| HDL-C | (mmol/L) | 1.45 | (0.378) | 1.38 | (0.382) | 1.29 | (0.379) |
| Triglycer- | (mmol/L) | 1.41 | (0.923) | 1.87 | (1.25) | 2.17 | (1.47) |
| ides | | | | | | | |
| FBG | (mmol/L) | 5.25 | (0.388) | 5.75 | (0.583) | 8 | (2.82) |
| PLG | (mmol/L) | 5.49 | (1.16) | 7.97 | (1.68) | 12.7 | (3.92) |

¹For quantitative variables, values shown are group means with standard deviations between parentheses.

### The AusDiab Cohort (Incident Disease)

Within the AusDiab cohort, 5,728 non-diabetic participants attended both a baseline and a five-year follow-up study, during which they were evaluated for prediabetes or diabetes. Demographic and clinical variables, as well as lipidomics data, were available at baseline for this group. Here, we selected participants that were non diabetic at both visits (n=5,510) or who were initially non-diabetic but were diagnosed with incident type 2 diabetes during the follow up period (n=218). Characteristics of the cohort are shown in Table 2.

### PE(P) Plasmalogen Alkenyl and Acyl Sidechain Composition

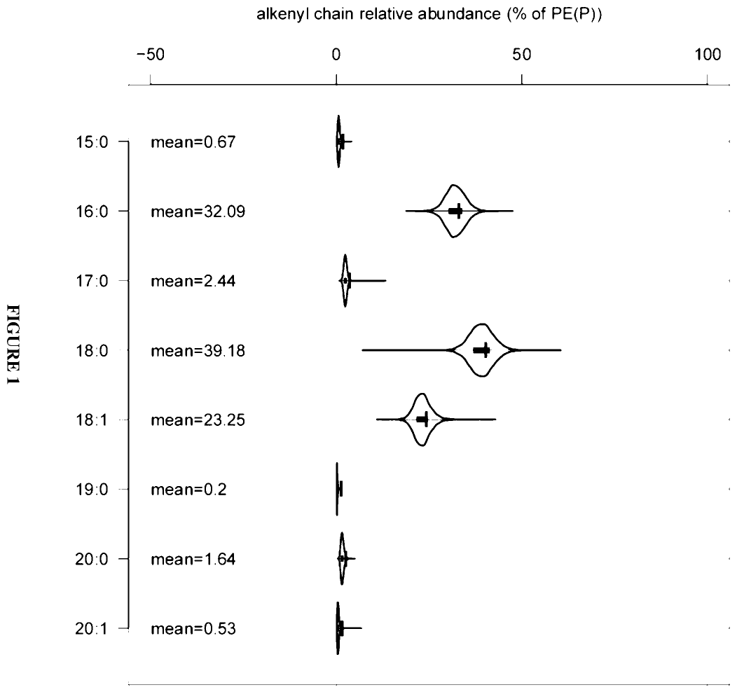

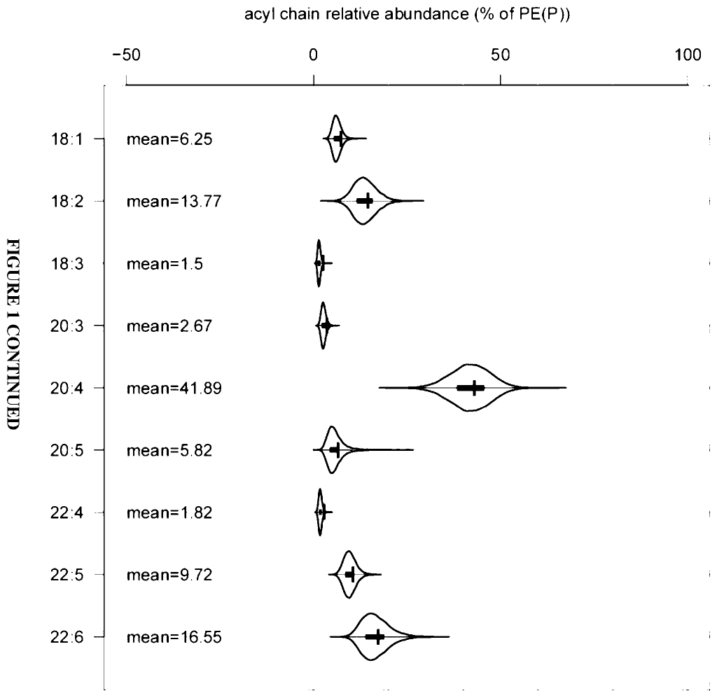

The typical plasma PE(P) alkenyl and acyl sidechain composition (expressed as relative abundances of each sidechain amongst all PE(P) lipids) across all participants from the AusDiab cohort are shown in FIG. 1. The most abundant PE(P) alkenyl chains are O-18:0 (39.18%), O-16:0 (32.09%), and O-18:1 (23.25%), while the most abundant acyl chains are 20:4 (41.89%), 22:6 (16.55%), and 18:2 (13.77%).

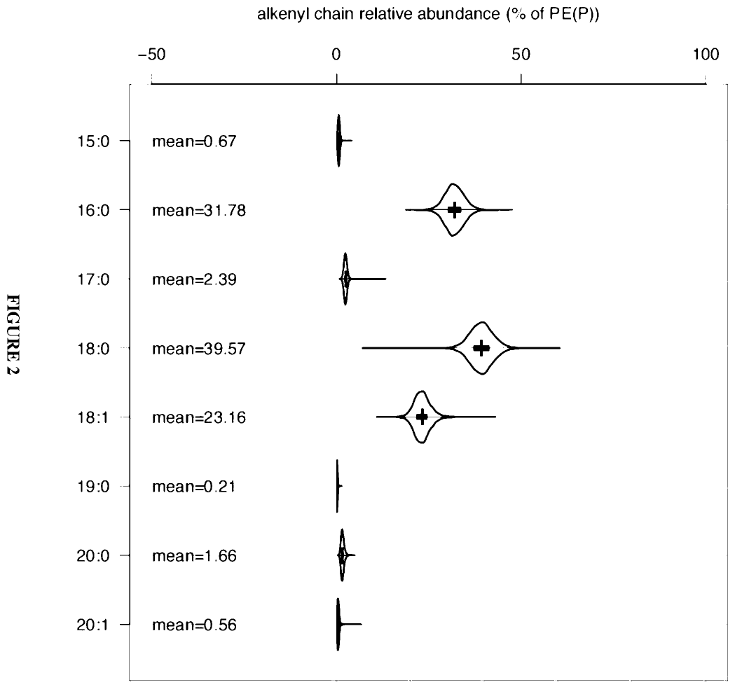

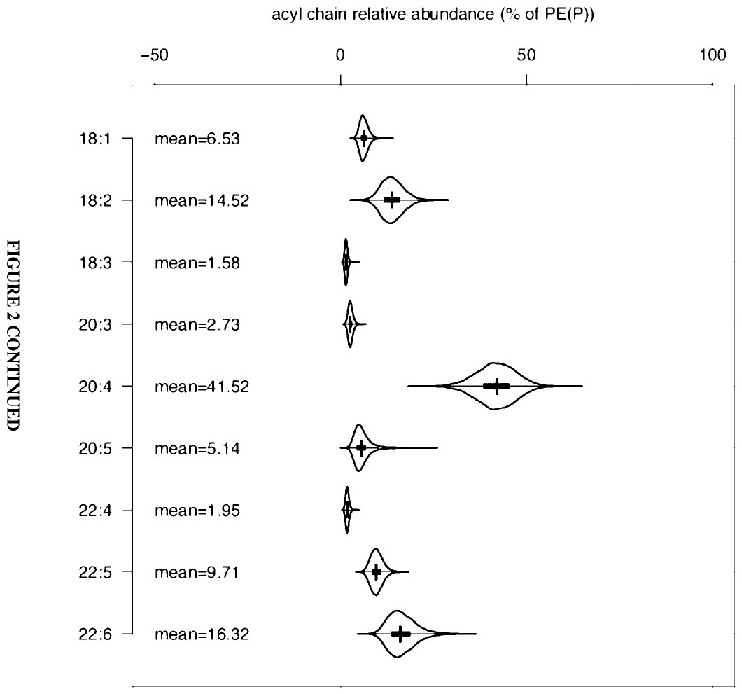

A subcohort of the AusDiab study representing “healthy” participants was also examined (FIG. 2, Tables 3 and 4). These were all participants who did not have diabetes and were between the ages of 25 and 34, BMI 20-25, FBG<6.0, 2h-PLG<7.8, total cholesterol<5.17 mM, triglycerides<1.68 mM. There were 519 participants in this group. The mean alkenyl and acyl % within the PE(P) class was similar to the total population although the variance was reduced in this subcohort.

### Ternary Diagrams Show No Obvious PE(P) Composition Differences Between Clinical Groups

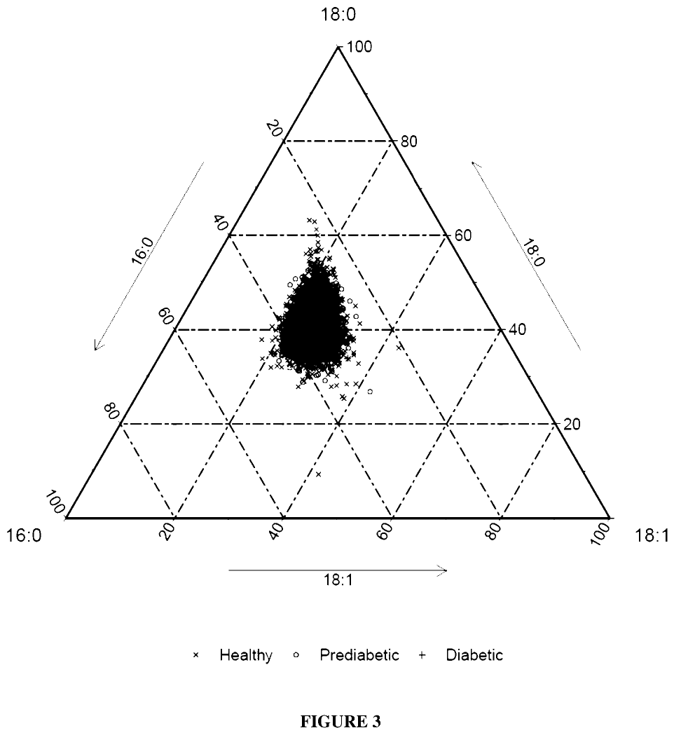

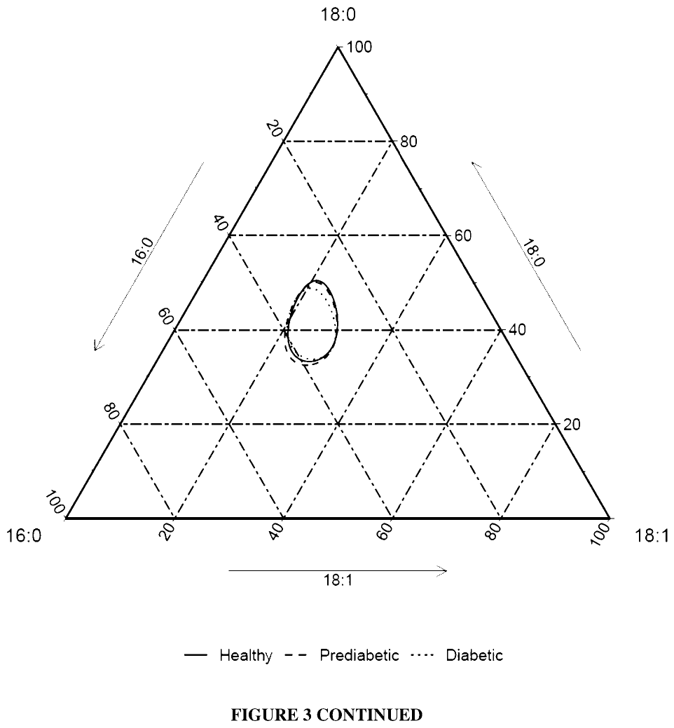

The alkenyl chain 8-part composition reported in FIG. 1 is translated here into the corresponding 3-part sub-composition (33.9% O-16:0; 41.4% O-18:0; 23.7% O-18:1, FIG. 3). A very tight alkenyl compositional distribution in each of the normoglycemic, prediabetes and diabetes groups was determined. There were differences between normoglycemic, prediabetes and diabetes groups (see FIG. 3).

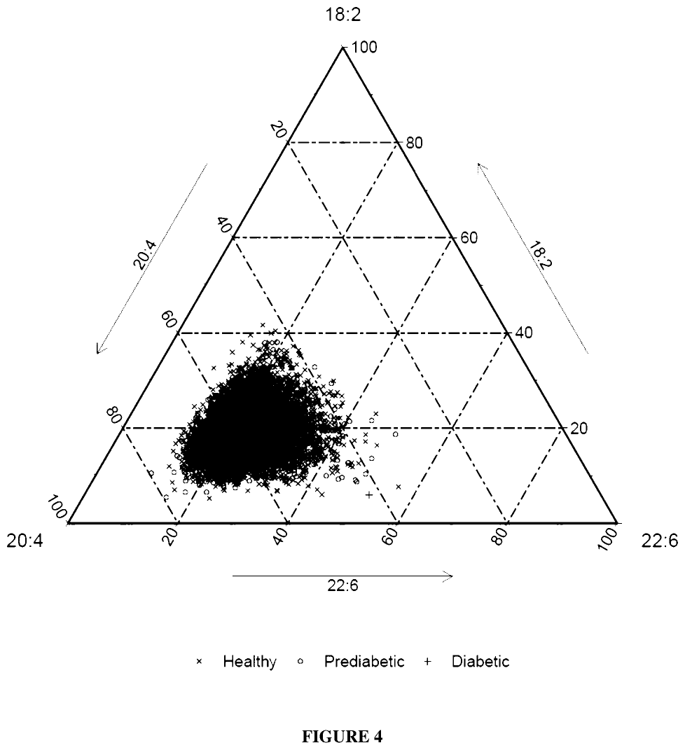

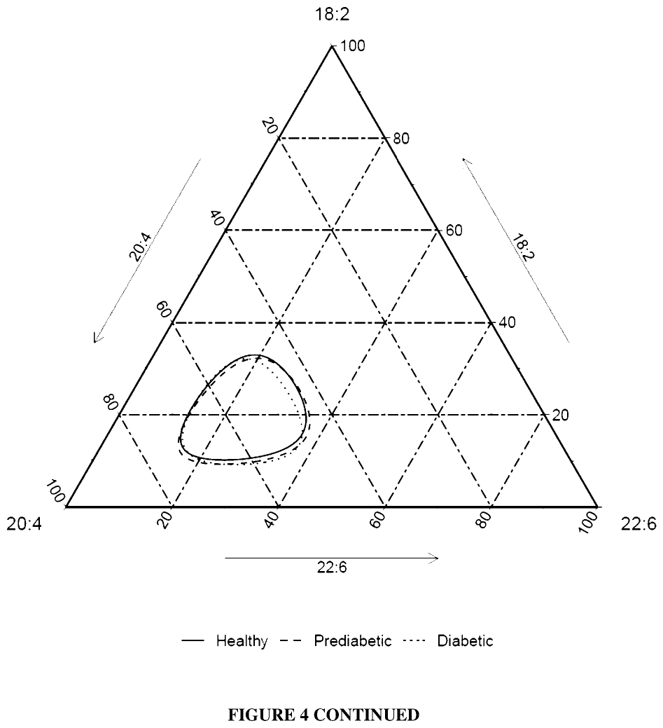

For the acyl chain composition (limited to the three most abundant acyl chains), again, there were compositional differences between groups (FIG. 4). However, the composition distribution shows greater variance than for the alkenyl chains (with a mean standard deviation of acyl proportions of 5.20% compared to a mean standard deviation of alkenyl proportions of 2.91%), showing that PE(P) alkenyl composition is more tightly regulated than the acyl composition.

Alkenyl and Acyl Chain Composition are Associated with Prevalent Diabetes

Investigating any potential associations between sidechain composition and health outcomes cannot rely solely on graphical observation. Accordingly, such associations were then analysed statistically, using logistic regression.

To assess the association of individual alkenyl and acyl chain proportions with diabetes, we performed separate logistic regressions using each of them in turn as predictor. These analyses were performed with the inclusion of traditional risk factors (sex, age and BMI) as covariates.

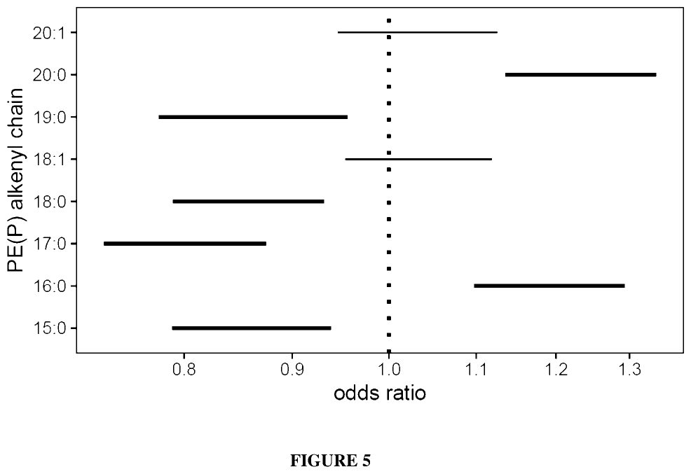

The proportion of the O-16:0 and O-20:0 alkenyl chains were found to be significant risk factors for disease, while the O-15:0, 0-17:0, 0-18:0 and O-19:0 alkenyl chain proportions were protective (FIG. 5). The effects of the top three alkenyl chains taken together act to cancel each other out, as would be expected as they are all inter-related by being parts of a composition.

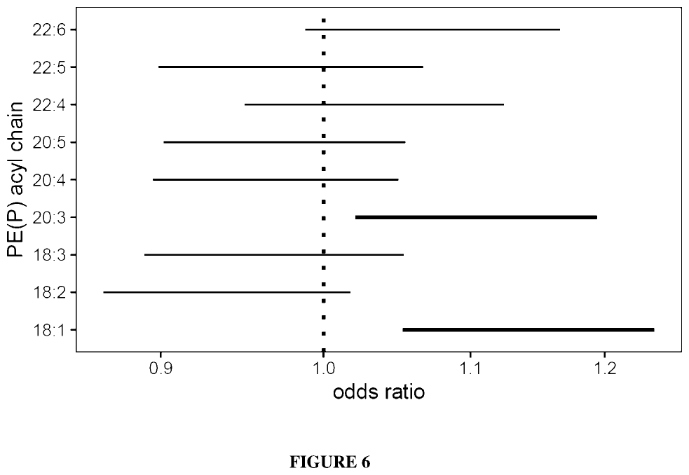

Using a similar model with acyl chain compositions, significant associations were found for 18:1 and 20:3 acyl chains with diabetes (FIG. 6).

Alkenyl and Acyl Chain Composition are Associated with Incident Diabetes

The AusDiab cohort comprised 5,510 non-diabetic individuals who remained non-diabetic after 5 years of follow up and 218 non-diabetic individuals at baseline who developed diabetes during the five year follow up period. Clinical characteristics (sex, age, BMI, total cholesterol, HDL and triglycerides) and lipidomics data are available for the initial visit, see Table 2. It was thus possible to explore lipid associations with incident diabetes within this cohort.

TABLE 2
Demographic and clinical description of
the AusDiab subcohort (incident diabetes)

| Demographic/Clinical | | | | Incident | |
|---|---|---|---|---|---|
| Variables¹ | | Control | | Diabetes | |
| N= | | 5510 | | 218 | |
| Sex | (% male) | 45% | | 51% | |
| Age | (years) | 50.7 | (12.6) | 55.7 | (12) |
| BMI | (kg/m²) | 26.6 | (4.57) | 29.5 | (5.6) |
| Total | (mmol/L) | 5.64 | (1.04) | 5.92 | (1.05) |
| cholesterol | | | | | |
| HDL-C | (mmol/L) | 1.45 | (0.38) | 1.33 | (0.404) |
| Triglycerides | (mmol/L) | 1.44 | (0.956) | 2.14 | (1.56) |
| FBG | (mmol/L) | 5.35 | (0.471) | 5.95 | (0.59) |
| PLG | (mmol/L) | 5.83 | (1.51) | 7.87 | (1.77) |

¹For quantitative variables, values shown are group means with standard deviations between parentheses.

TABLE 3
Alkenyl chain composition of the healthy
subcohort of the AusDiab study

| Alkenyl | Mean | SD | Confidence region |
|---|---|---|---|
| chain | (%) | (%) | (Mean % +/− 2 StDev) |
| 15:0 | 0.67 | 0.22 | 0.23-1.11 |
| 16:0 | 32.09 | 2.66 | 26.77-37.41 |
| 17:0 | 2.44 | 0.45 | 1.54-3.34 |
| 18:0 | 39.18 | 3.29 | 32.6-45.76 |
| 18:1 | 23.25 | 2.32 | 18.61-27.89 |
| 19:0 | 0.2 | 0.07 | 0.06-0.34 |
| 20:0 | 1.64 | 0.43 | 0.78-2.5 |
| 20:1 | 0.53 | 0.29 | 0-1.16 |

TABLE 4
Acyl chain composition of the healthy
subcohort of the AusDiab study

| Acyl | Mean | SD | Confidence region |
|---|---|---|---|
| chain | (%) | (%) | (Mean % +/− 2 StDev) |
| 18:1 | 6.25 | 1.14 | 3.97-8.53 |
| 18:2 | 13.77 | 3.08 | 7.61-19.93 |
| 18:3 | 1.5 | 0.38 | 0.74-2.26 |
| 20:3 | 2.67 | 0.64 | 1.39-3.95 |
| 20:4 | 41.89 | 5.32 | 31.25-52.53 |
| 20:5 | 5.82 | 2.23 | 1.36-10.28 |
| 22:4 | 1.82 | 0.45 | 0.92-2.72 |
| 22:5 | 9.72 | 1.65 | 6.42-13.02 |
| 22:6 | 16.55 | 3.65 | 9.25-23.85 |

Following the same strategy as above, logistic regression was performed to investigate the association of alkenyl chain composition with incident diabetes (adjusting for age, sex and BMI).

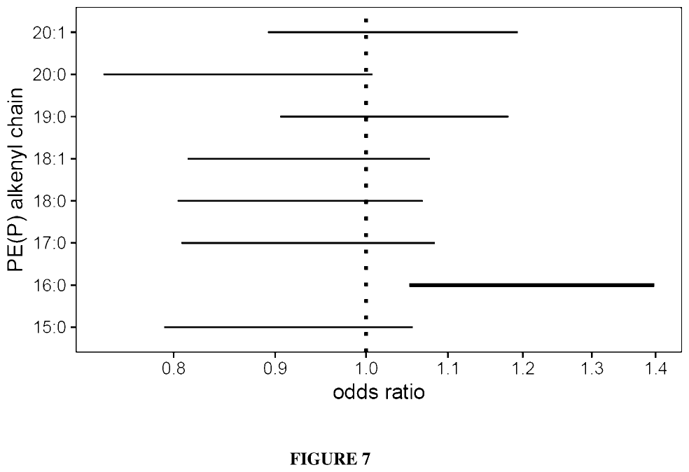

A significant positive association was observed of the O-16:0 alkenyl chain with incident diabetes, while O-18:0 and O-18:1 alkenyl chains showed non-significant negative trends (FIG. 7).

These results obtained in an incident diabetes setting confirmed those observed in the prevalent prediabetes/diabetes setting: of the plasma PE(P) alkenyl chains, the relative abundance of O-16:0 appears as a strong risk factor for metabolic disease.

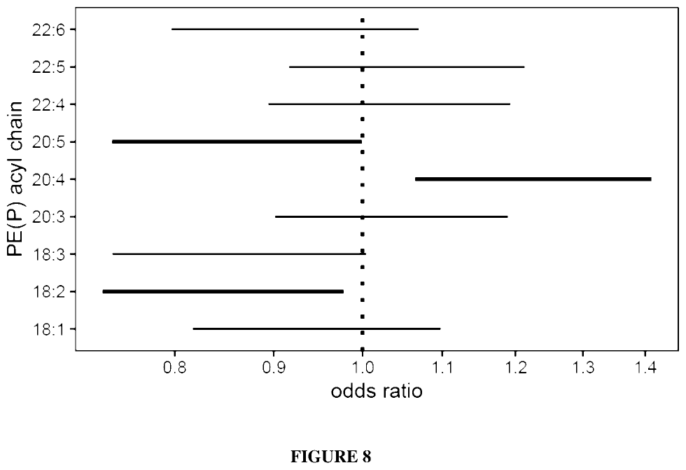

The association of acyl chain composition with incident diabetes were quite different to those with prevalent diabetes (FIG. 8). Indeed, 20:4 was found to be a risk factor, while 18:2 is found to be protective against incident diabetes.

### EXAMPLE 2

### Shark Liver Oil Supplementation in Overweight/Obese Men

A supplementation study was designed to evaluate the impact of plasmalogen precursor supplementation (shark liver oil, SLO) in overweight/obese males with features of metabolic syndrome. This study was a randomized, double-blind, placebo-controlled (methylcellulose) cross-over study. The study population consisted of 10 males from 25-60 years of age with BMI in the range of 28-40 kg/m2. The participants had no evidence of diabetes or cardiovascular disease, were not taking any lipid-lowering or antihypertensive medication, and were not taking any fish oil supplementation. The participants had normal liver function.

### Study Design

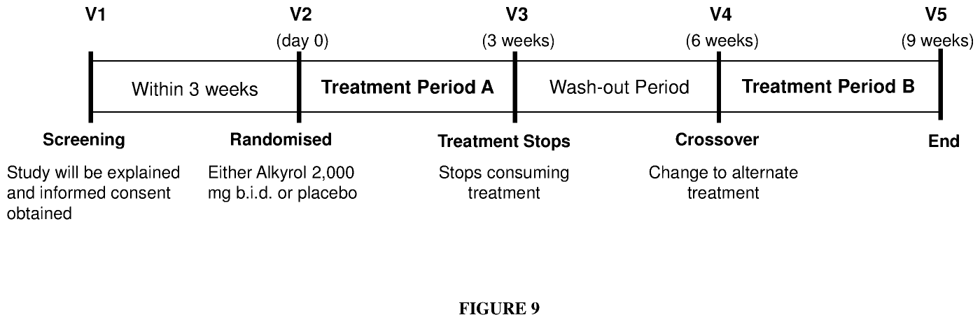

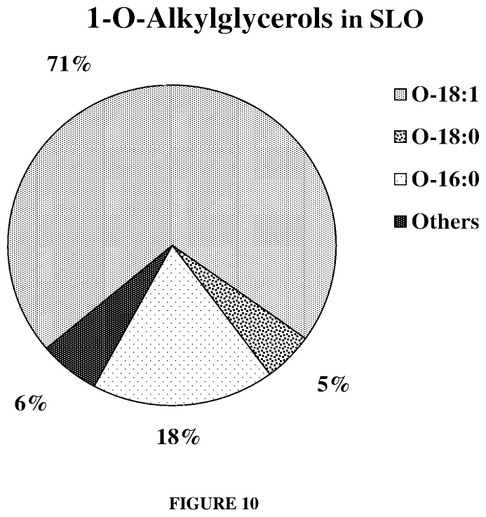

To assess whether and how plasmalogen levels are modulated by dietary supplementation of alkyl-diacylglycerols, with the intent of resolving key features of metabolic syndrome, a phase 0/I trial of alkylglycerol supplementation was performed in overweight and obese males. In this double-blind, placebo-controlled crossover study, participants (n=10) were overweight or obese (BMI 28-40) males (aged 25-60 years) with no signs of cardiovascular disease or diabetes. Participants were randomised into placebo or treatment arms. They received 4 g Alkyrol® (shark liver oil enriched in alkyl-diacylglycerols) per day or placebo for 3 weeks followed by a 3-week washout phase, and then were crossed over to 3 weeks of the alternate placebo/Alkyrol® treatment. Blood was collected at the time of screening and at the start and end of each intervention (FIG. 9).

### Whole Blood Separation

Patients' blood samples were collected in K3 EDTA tubes. They were then centrifuged at 3000 RPM (1,711 g) on a Heraeus multifuge 1S-R for 15 min at room temperature to separate the blood into its components. The first centrifuge separated the blood into three layers; a cloudy WBC buffy layer in the middle with plasma above and red cells below. The top plasma layer was aspirated and 1 μL of 100 mM BHT per ml plasma was added and the plasma stored at −80° C. The buffy layer was transferred to another tube and mixed with phosphate buffered saline (PBS) until it reached 1 cm from top of tube. It was then layered on top of Ficoll-Paque and centrifuged at 400 g for 30 min with lowest brake at room temperature. The resulting upper layer (containing the plasma and platelets) was discarded and the thin cloudy layer of white blood cells was collected and transferred to a fresh tube. PBS was added and the sample centrifuged at 250 g for 10 min with highest brake. The sample was then resuspended in PBS and centrifuged at 100 g for 10 min to obtain the white blood cell pellet. The pellet was then stored at −80° C. To obtain the red blood cell membrane, the red blood cell layer separated at the initial centrifugation was added with PBS and centrifuged at 1,700×g for 10 min. The samples were then added with deionised water and centrifuged at 14,800×g for 10 min. The red blood cell membrane pellet obtained was then stored at −80° C.

Flow Cytometry

There are 3 types of monocyte subpopulations of interest, which are the classical, intermediate and non-classical monocyte subsets. They are identified by CD 56, CD2, CD 19, NKp46, CD 15, HLA DR, CD 14 and CD 16 antibody expression analysis on the Canto II flow cytometer.

To assess the monocyte subpopulation, 100 μL of whole blood from each patient visit was added to 5 mL of Lysis Buffer lx from BD PharmLyse and was then incubated in the dark for 5 mins. The sample was then added to 10 mL wash buffer (9:1 ratio of PBS and fetal bovine serum) and centrifuged at 300 xg for 5 min at 4° C. The resulting pellet was then resuspended in wash buffer, placed in an Eppendorf tube and centrifuged at 300×g for 5 min at room temperature. 50 μL of the sample was divided into 5 clean Eppendorf tubes and mixed with wash buffer. Antibodies were added to the Eppendorf tubes to bind to the monocytes. They were then incubated on ice for 30 min in the dark. The samples were then washed with PBS and centrifuged at 300xg before transferring the cells to a FACS tube. The samples were then run on the Canto II flow cytometer and analysed by the BD FACS Diva Software.

### Statistical Analysis

The mean % change of the clinical measurements, whole blood count, inflammatory markers, monocyte subsets population in the treatment and placebo groups were also calculated and were compared with repeated measures analysis of variance (ANOVA), taking into account treatment (as a between-subjects variable) and treatment order. P-values less than 0.05 was considered as significant.

The mean % change of plasma lipid class concentrations between Alkyrol® and placebo treatments in the two intervention arms (visit 2 to 3 and visit 4 to 5) were also compared using repeated measures ANOVA. The class data was also normalized to phosphatidylcholine (PC) to account for variations in lipoprotein particles. A similar repeated measures ANOVA was performed on alkenyl chain proportions amongst plasma PE(P) lipids to determine which parts of the alkenyl composition were affected by SLO supplementation.

### Results

### Shark Liver Oil Composition

Shark liver oil (SLO) contains high concentrations of plasmalogen precursors (1-O-alkyl, 2,3-diacylglyerol), with a different alkyl composition to that found in human plasma plasmalogens.

The composition of the 1-O-alkyl groups on the SLO was found to be predominantly O-18:1 (71%), O-16:0 (18%), and O-18:0 (5%) (FIG. 9). This is at odds to the usual PE(P) alkenyl chain composition found in human blood (see Example 1) that typically contains O-16:0, 33.9%; O-18:0 41.4%; O-18:1 23.7%; and other alkenyl chains 7%).

### Participant Cohort

This study consisted of 10 male participants and was conducted between December 2015 and August 2016. Table 5 shows the baseline characteristics of the participants. No side effects were reported for any participants when treated with shark liver oil.

TABLE 5
Baseline characteristics of the participant cohort

| Characteristic | Mean ± Standard Deviation |
|---|---|
| Age (years) | 50 ± 10 |
| BMI (kg/m²) | 32 ± 3 |
| Waist/Hip Ratio | 0.96 ± 0.05 |
| Systolic Blood Pressure (mmHG) | 116 ± 13 |
| Diastolic Blood Pressure (mmHG) | 74 ± 10 |
| Heart Rate (bpm) | 57 ± 8 |
| Total Cholesterol (mmol/L) | 5.39 ± 1.19 |
| High Density Lipoprotein (mmol/L) | 1.09 ± 0.12 |
| Low Density Lipoprotein (mmol/L) | 3.32 ± 0.86 |
| Triglycerides (mmol/L) | 2.14 ± 1.08 |
| Fasting Glucose (mmol/L) | 4.90 ± 0.48 |

n = 10 male participants

### The Effect of Alkylglycerol Supplementation on Clinical Measures

As shown in Table 6, there were significant decreases in the levels of cholesterol (−7%) and triglycerides (22%). However, there were no significant changes in the fasting glucose, HbA1c and lipoproteins in the treatment group compared to placebo.

TABLE 6
Effects of alkylglycerol supplementation on clinical measures

| | Mean | Mean | | Mean | Mean | | |
|---|---|---|---|---|---|---|---|
| | pre- | post- | Placebo | pre- | post- | Treatment | |
| | placebo | placebo | mean % | treatment | treatment | mean % | |
| Parameter¹ | value² | value² | change² | value² | value² | change² | P-value³ |
| FPG² | 5.01 ± | 5.02 ± | 0.20 ± | 5.10 ± | 5.08 ± | −0.05 ± | 0.825 |
| (mmol/L) | 0.12 | 0.15 | 1.77 | 0.16 | 0.17 | 2.67 | |
| HbA1c (%) | 5.48 ± | 5.48 ± | -0.01 ± | 5.46 ± | 5.47 ± | 0.16 ± | 0.856 |
| | 0.06 | 0.09 | 1.00 | 0.05 | 0.09 | 1.29 | |
| Cholesterol | 5.35 ± | 5.49± | 3.07 ± | 5.36 ± | 4.98 ± | −7.04 ± | 0.006 |
| (mmol/L) | 0.34 | 0.35 | 2.09 | 0.34 | 0.36 | 2.72 | |
| HDL-C | 1.17 ± | 1.20 ± | 3.54 ± | 1.13 ± | 1.14 ± | 1.53 ± | 0.594 |
| (mmol/L) | 0.06 | 0.06 | 3.16 | 0.05 | 0.06 | 4.75 | |
| LDL-C | 3.26 ± | 3.29 ± | 2.28 ± | 3.20 ± | 3.08 ± | −3.50 ± | 0.371 |
| (mmol/L) | 0.26 | 0.22 | 3.08 | 0.23 | 0.25 | 4.02 | |
| Triglyceride | 2.01 ± | 2.18 ± | 8.29 ± | 2.25 ± | 1.64 ± | −21.77 ± | 0.023 |
| (mmol/L) | 0.28 | 0.38 | 8.05 | 0.39 | 0.35 | 8.80 | |

¹FPG, fasting plasma glucose; HbA1c, glycated hemoglobin; HDL-C, high density lipoprotein cholesterol; LDL-C, low density lipoprotein cholesterol
²Data is presented in the form of mean ± SEM.
³Significance was determined using Repeated Measures ANOVA; p-values less than 0.05 are in bold

### The Effect of Alkylglycerol Supplementation on the Plasma Lipidome

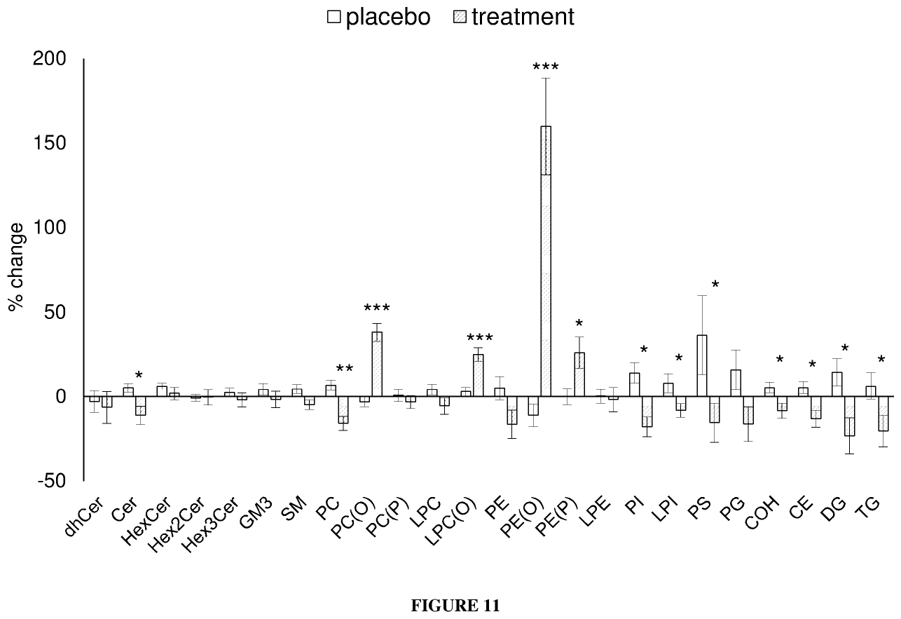

The supplementation of shark liver oil resulted in significant differences in post-intervention changes between treatment and placebo groups for 13 lipid classes, shown in FIG. 11. Foremost, there was a significant 160% increase of alkylphosphatidylethanolamine (PE(O)) in the treatment group compared to the placebo group (P<0.001). Furthermore, the levels of lysoalkylphosphatidylcholine (LPC(O)), alkylphosphatidylcholine (PC(O)) and alkenylphosphatidylcholine (PC(P)) also increased significantly (P<0.05) more in the treatment group (25%, 38% and 26% respectively) than in the placebo group (3%, -3% and 0% respectively). We also note that the level of phosphatidylcholine (PC) decreased significantly (P<0.05) by 16% in the treatment group.

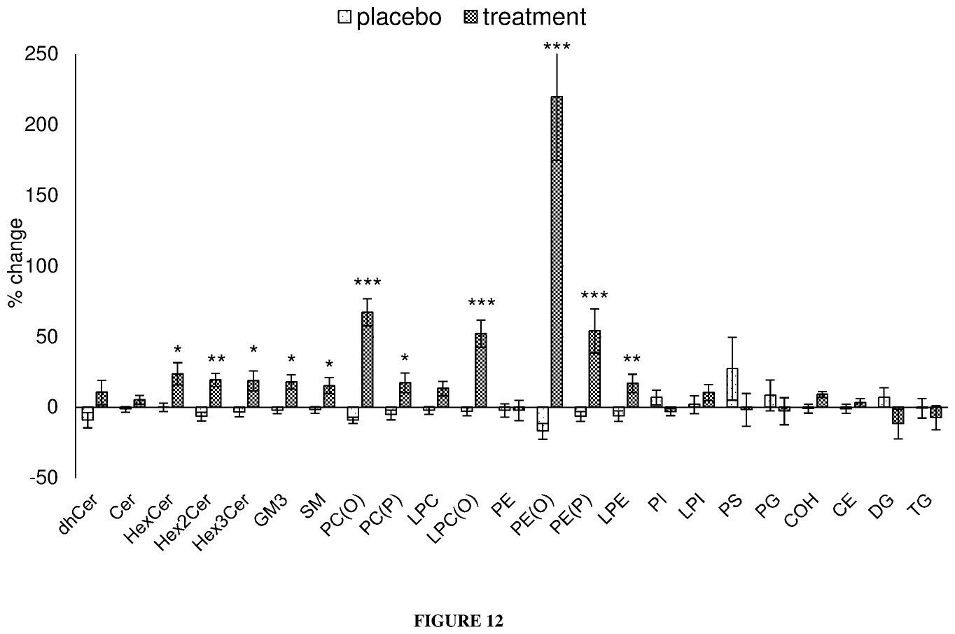

The levels of total PC are of particular interest in this analysis. Indeed, phosphatidylcholine is the major phospholipid making up the surface layer of all lipoprotein particles, and its decrease following SLO supplementation indicates a decrease in the levels of total circulating plasma lipids. In order to assess the relative change in plasmalogens and ether lipid classes relative to this variation, we normalised the lipid data to total PC and reiterated the repeated-measures ANOVA analysis on these data (FIG. 12). Eleven lipid classes showed a significant difference between the response to SLO relative to placebo.

The increase in PE(P) levels is even more notable after accounting for decreasing total lipoprotein, indicating a strong effect. Having detected such a strong effect on PE(P) levels, the inventor explored whether the sidechain composition of PE(P) lipids was also affected.

### The Effect of Alkylglycerol Supplementation on Plasma PE(P) Sidechain Composition

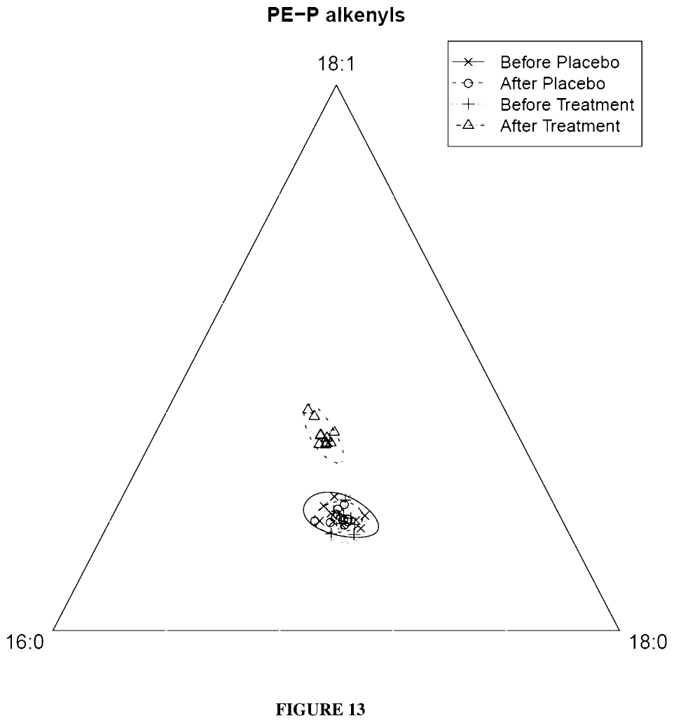

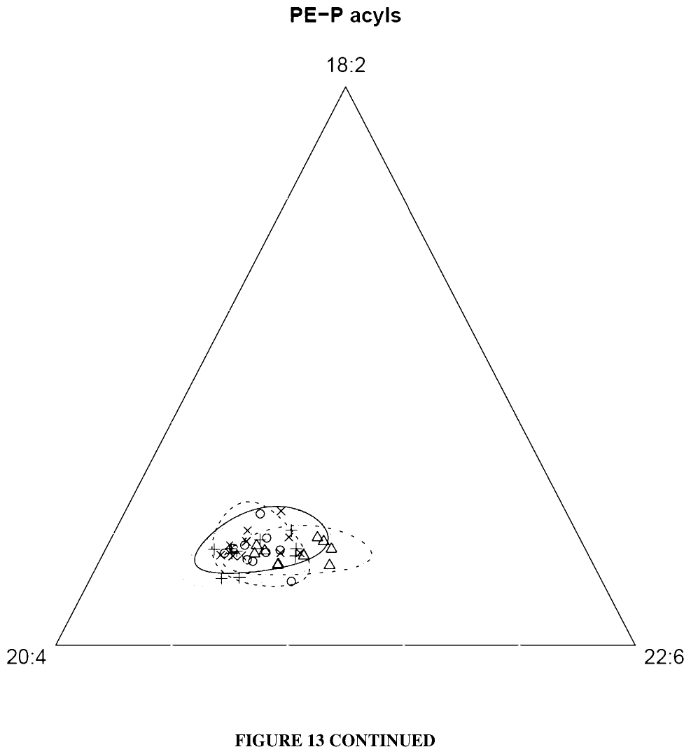

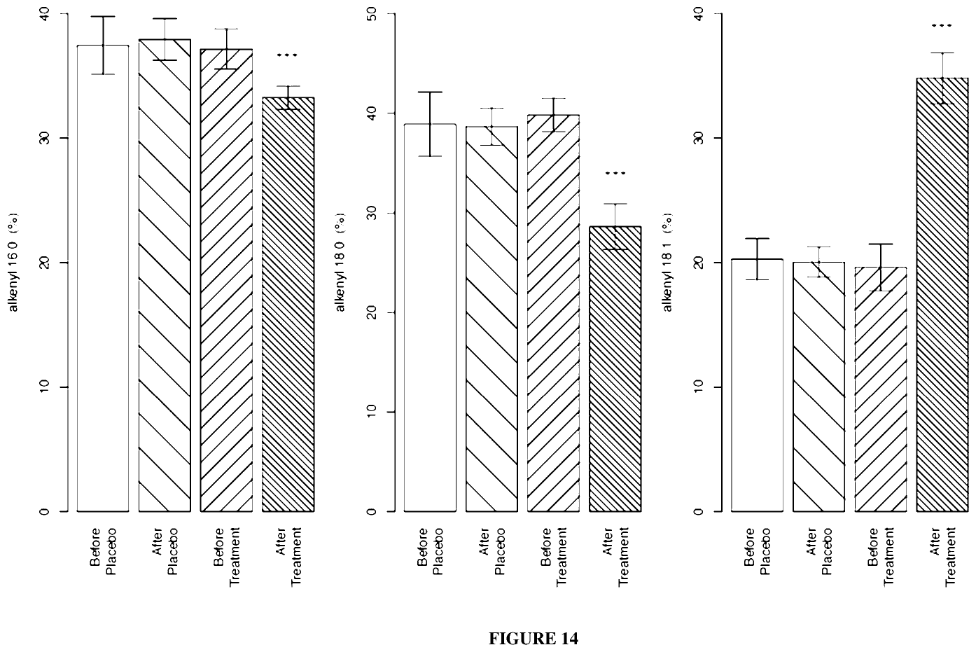

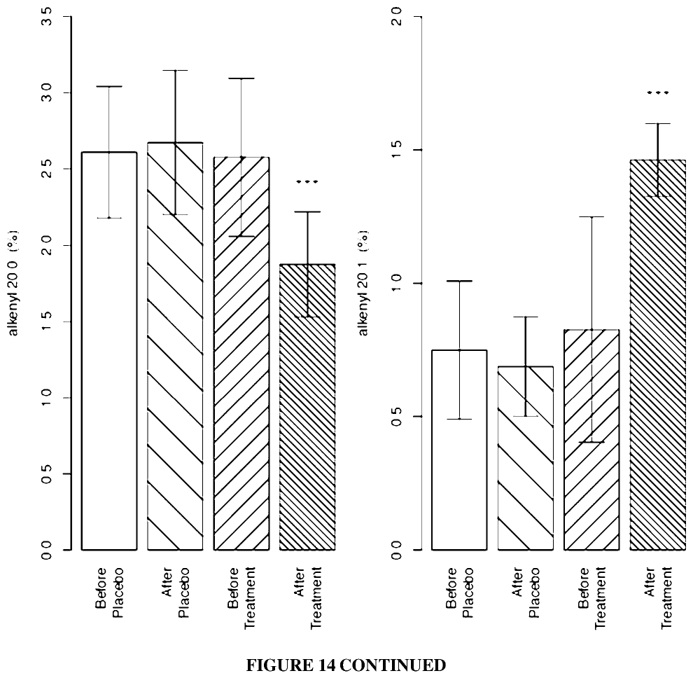

Indeed, supplementation with SLO changes plasma PE(P) alkenyl composition in humans (FIG. 13). The changes were observable in all participants having received SLO supplementation, irrespective of intervention order, again illustrating that the wash-out period was sufficient.

It is immediately obvious that SLO supplementation substantially increases the proportion of 18:1 amongst PE(P) alkenyl chains, while having no impact on the top acyl chains. Looking more closely into the relative abundances of all 5 alkenyl chains available in this study (FIG. 14), it is apparent to the inventors that the increase in O-18:1 (+72%, from ˜20% to ˜35%) comes at the expense of O-16:0 (-11%), O-18:0 (-28%) and O-20:0 (−26%). Interestingly, the levels of O-20:1, though low, also seem to increase (+87%, from ˜0.75% to ˜1.5%). All changes are nominally statistically significant (p<<0.05).

The inventor determined that as the 3-week wash-out period was sufficient for PE(P) side chain proportions to return to normal or near-normal, alkenyl composition must not only be tightly controlled, but also dynamically controlled. Having explored the effect of SLO supplementation on the lipidome, systemic immune effects of the lipidome changes induced by SLO supplementation were also determined.

### The Effect of Alkylglycerol Supplementation on the Whole Blood Count

There was a significant decrease of 5% in the level of white blood cells (Table 7) in the treatment group compared to placebo. The decrease is especially significant in the level of neutrophils (10%). The other measures of the whole blood count showed no significant difference between the treatment and placebo group.

TABLE 7
Effects of alkylglycerol supplementation on whole blood count

| | Mean | Mean | | Mean | Mean | | |
|---|---|---|---|---|---|---|---|
| | pre- | post- | Placebo | pre- | post- | Treatment | |
| | placebo | placebo | mean % | treatment | treatment | mean % | |
| Parameter¹ | value² | value² | change² | value² | value² | change² | P-value³ |
| Hb (g/L) | 150 ± | 149 ± | −0.87 ± | 148 ± | 145 ± | −2.00 ± | 0.524 |
| | 2.46 | 2.42 | 1.20 | 2.37 | 1.96 | 1.12 | |
| WBC | 6.05 ± | 6.43 ± | 5.33 ± | 6.45 ± | 5.82 ± | −9.05 ± | 0.031 |
| (10⁹/L) | 0.26 | 0.51 | 5.00 | 0.35 | 0.37 | 4.66 | |
| Platelets | 212 ± | 211 ± | 0.37 ± | 213 ± | 201 ± | −4.85 ± | 0.268 |
| (10⁹/L) | 12.74 | 10.96 | 2.99 | 12.27 | 8.83 | 1.93 | |
| RBC | 4.91 ± | 4.87 ± | −0.95 ± | 4.83 ± | 4.71 ± | −2.27 ± | 0.479 |
| (10¹² L) | 0.10 | 0.10 | 1.19 | 0.10 | 0.08 | 1.29 | |
| Hct (L/L) | 0.45 ± | 0.44 ± | −1.53 ± | 0.44 ± | 0.43 ± | −1.69 ± | 0.914 |
| | 0.01 | 0.01 | 1.03 | 0.01 | 0.00 | 1.32 | |
| Neutrophils | 3.38 ± | 3.78 ± | 10.07 ± | 3.65 ± | 3.26 ± | −9.68 ± | 0.021 |
| (10⁹/L) | 0.19 | 0.38 | 6.03 | 0.29 | 0.31 | 5.69 | |
| Lymphocytes | 1.99 ± | 1.92 ± | −2.93 ± | 2.03 ± | 1.90 ± | −4.63 ± | 0.571 |
| (10⁹/L) | 0.11 | 0.12 | 3.89 | 0.13 | 0.09 | 5.16 | |
| Eosinophils | 0.15 ± | 0.17 ± | 8.00 ± | 0.16 ± | 0.15 ± | 2.93 ± | 0.398 |
| (10⁹/L) | 0.01 | 0.02 | 7.55 | 0.02 | 0.01 | 12.33 | |
| Basophils | 0.04 ± | 0.04 ± | 10.67 ± | 0.05 ± | 0.04 ± | −2.62 ± | 0.152 |
| (10⁹/L) | 0.00 | 0.00 | 5.06 | 0.01 | 0.00 | 8.48 | |

¹Hb, hemoglobin; WBC, white blood cell; Hct, hematocrite; RBC, red blood cell
²Data is presented in the form of mean ± SEM.
³Significance was determined using Repeated Measures ANOVA; p-values less than 0.05 are in bold

### The Effect of Alkylglycerol Supplementation on Inflammatory Markers

As shown in Table 8, high sensitivity C-reactive protein (hsCRP), an inflammatory marker, was found to be significantly reduced by 28% in the treatment group compared to control. However, the other inflammatory cytokines (TNFa, MCP-1 and VCAM-1) were not shown to be significantly different in the treatment group compared to placebo.

TABLE 8
Effects of alkylglycerol supplementation on inflammatory markers

| | Mean | Mean | | Mean | Mean | | |
|---|---|---|---|---|---|---|---|
| | pre- | post- | Placebo | pre- | post- | Treatment | |
| | placebo | placebo | mean % | treatment | treatment | mean % | |
| Parameter¹ | value² | value² | change² | value² | value² | change² | P-value³ |
| hsCRP | 1.86 ± | 2.20 ± | 15.69 ± | 2.70 ± | 1.62 ± | −27.78 ± | 0.048 |
| (mg/L) | 0.29 | 0.42 | 13.12 | 0.49 | 0.17 | 8.81 | |
| TNFa | 3.38 ± | 3.16 ± | −1.84 ± | 3.52 ± | 3.27 ± | −7.39 ± | 0.926 |
| (pg/mL) | 0.45 | 0.30 | 11.10 | 0.47 | 0.53 | 6.06 | |
| MCP-1 | 148.57 ± | 146.07 ± | −0.54 ± | 142.55 ± | 156.48 ± | 12.73 ± | 0.244 |
| (pg/mL) | 9.23 | 8.52 | 4.15 | 10.28 | 11.93 | 10.15 | |
| VCAM-1 | 926.16 ± | 881.66 ± | −4.77 ± | 867.43 ± | 870.04 ± | 1.59 ± | 0.310 |
| (ng/mL) | 72.99 | 74.18 | 2.66 | 83.42 | 81.14 | 6.08 | |

¹hsCRP, high sensitive c-reactive protein; TNFa, tumor necrosis factor alpha; MCP-1, monocyte chemoattractant protein-1; VCAM-1, vascular cell adhesion protein 1
²Data is presented in the form of mean ± SEM.
³Significance was determined using Repeated Measures ANOVA; p-values less than 0.05 are in bold

### The Effect of Alkylglycerol Supplementation on Monocyte Subsets Populations

There was no significant change in the total monocyte count after treatment (Table 9). Out of the monocyte subsets, only the intermediate subset was found to be significantly decreased in the treatment group compared to the placebo group. However, when looking at the monocyte subsets as a percentage of total monocytes, the intermediate subset was not found to be significantly decreased.

TABLE 9
Effects of alkylglycerol supplementation on monocyte subset populations

| | | Mean | | Mean | | |
|---|---|---|---|---|---|---|
| | | pre-placebo | Mean | pre-treatment | Mean | |
| Parameter | | value¹ | change¹ | value¹ | change¹ | P-value² |
| Monocytes | | 0.49 ± 0.03 | −0.01 ± 0.05 | 0.57 ± 0.04 | −0.10 ± 0.03 | 0.121 |
| (10⁹/L) | | | | | | |
| Monocyte | Classical | 0.36 ± 0.04 | −0.04 ± 0.04 | 0.40 ± 0.05 | −0.05 ± 0.03 | 0.851 |
| Subsets | Intermediate | 0.021 ± 0.003 | 0.005 ± 0.004 | 0.043 ± 0.009 | −0.023 ± 0.009 | 0.035 |
| (10⁹/L) | Non-Classical | 0.11 ± 0.01 | 0.03 ± 0.03 | 0.13 ± 0.02 | −0.02 ± 0.02 | 0.262 |
| Monocyte | Classical | 72.78 ± 3.23 | −6.15 ± 5.06 | 66.93 ± 6.29 | 4.53 ± 5.54 | 0.187 |
| Subsets | Intermediate | 4.23 ± 0.68 | 1.59 ± 0.69 | 7.09 ± 1.43 | −2.83 ± 1.47 | 0.052 |
| (% of total | Non-Classical | 23.02 ± 3.29 | 4.53 ± 5.05 | 25.68 ± 6.91 | −0.06 ± 5.93 | 0.598 |
| monocytes) | | | | | | |

¹Data is presented in the form of mean ± SEM.
²Significance was determined using Repeated Measures ANOVA; p-values less than 0.05 are in bold

### Effect of Alkylglycerol Supplementation on Plasmalogens and Other Lipid Classes

This is the first study to look at the effect of alkylglycerol supplementation on the level of circulating ether lipids, including plasmalogens, in humans.

In plasma, SLO supplementation led to a nominally significant increase of PC(O), PE(O) and LPC(O) by 38%, 160% and 25%, respectively (FIG. 11). In particular, there is also a nominally significant 26% increase of PE(P). The distribution of these lipid classes can be explained by looking at the plasmalogen biosynthesis pathway. PE(O) and PC(O) are formed from the addition of either ethanolamine or choline respectively to alkyl-acyl-glycerol. PC(O) cannot to be converted directly to choline plasmalogen (PC(P)), which explains the build-up of PC(O) in plasma. PE(O) is converted to alkenylphosphatidylethanolamine (PE(P)) first in the pathway, and is then transformed into PC(P). Our data suggest that the enzymes responsible for the conversion from PE(O) to PC(P) are limited in the liver, leading to a build-up of PE(O) in plasma.

An overall decrease in lipids was observed, as evidenced by the nominally significant 16% decrease in phosphatidylcholine (PC), the most abundant phospholipid, and proxy for lipoprotein concentration. Interestingly, there were concomitant decreasing trends of plasma ceramides (Cer), phosphatidylethanolamine (PE), phosphatidylinositol (PI), lysophosphatidylinositol (LPI), phosphatidylserine (PS), phosphatidylglycerol (PG), free cholesterol (COH), cholesteryl ester (CE), diacylglycerol (DG) and triglycerides (TG), though not all were nominally significant (FIG. 11). Correcting for decreasing lipoprotein levels eliminated this effect (FIG. 12), showing that these decreases were linked to overall lipoprotein levels, while strengthening the observed increases in PE(P), PE(O), PC(O) and LPC(O). Taken together, this suggests that the supplementation with alkylglycerol can reduce the dyslipidaemia, which is a significant risk factor for cardiometabolic disease.

### Effect of Alkylglycerol Supplementation on PE(P) Sidechain Composition

The strong increase in overall PE(P) lipids despite decreasing lipoproteins led us to investigate the effects of supplementation on PE(P) sidechain composition. The alkenyl chain composition of PE(P) lipids was indeed modified following SLO supplementation, with gains in O-18:1 (and O-20:1) at the expense of other chains (FIG. 14). This is of therapeutic interest; indeed, in Example 1 the relative abundance of alkenyl chain O-18:1 was not associated with diabetes, however, a higher proportion of O-16:0 was a risk factor for diabetes. Increasing the proportion of O-18:1 via supplementation is proposed to reduce O-16:0 and thereby the risk of diabetes. This raises the possibility of formulation of an alkylglycerol supplement designed to provide a specific composition of PE(P) species to optimise the health benefits of the supplementation.

### Effect of Alkylglycerol Supplementation on Clinical Measures

The supplementation of alkylglycerol decreased cholesterol and triglycerides in this study. This supports the trend of decreasing dyslipidemia observed in the plasma lipidomic profile. Dyslipidemia is responsible for 50% of attributable risk for the causation of acute myocardial infarction (Yusuf S, et al. Lancet. 2004; 364(9438):937-52.). As a consequence, lipid disorders account for significant health care expenditure including via the Pharmaceutical Benefits Scheme (PBS).

Other clinical measures showed no significant changes with the 3-week supplementation of shark liver oil, though their trends show beneficial effects to health (decreased fasting glucose, HbA1c and LDL-Cholesterol with increased HDL-Cholesterol).

### Effect of Supplementation of Alkylglycerol on Whole Blood Count

The supplementation of alkylglycerol decreased the total number of white blood cells, particularly the neutrophils group. Neutrophils are the most abundant group of white blood cells, which play a role in the innate immune system. They are produced in the bone marrow and are recruited to the site of trauma within minutes. Neutrophils are widely recognized as prothrombotic as they cause platelet adhesion, activation, aggregation, mechanisms which are risk factors for thrombus (Caielli S, Curr Opin Immunol. 2012; 24(6):671-7.). The decrease in neutrophils can reduce inflammatory responses and thereby the formation of thrombus, in turn decreasing the risk of atherosclerosis (Paoletti R, Circulation. 2004; 109(23 Suppl 1):III20-6.). Other measures of the whole blood count showed no significant changes.

### Effect of Supplementation of Alkylglycerol on Monocyte Subsets

Inflammation plays a major role in the pathogenesis of atherosclerosis (Paoletti R supra). In particular, monocytes migrate from circulation to sites of injury to differentiate into macrophages, notably colonising atherosclerotic plaque. Monocytes are categorized into 3 subsets with different functions. Classical monocytes are involved in the process of phagocytosis; non-classical monocytes are patrolling immune cells that secrete inflammatory cytokines upon encountering a foreign body, while the intermediate monocytes are both phagocytic and inflammatory in nature.

In this study, the absolute count of intermediate monocytes decreased significantly, and when expressed as a percentage of total monocytes, also showed a decrease though not significant (p=0.052). This suggests that the supplementation of alkylglycerol can shift the distribution of monocytes to a less inflammatory state in humans, which may have beneficial effects on inflammatory diseases such as atherosclerosis.

### Effect of Supplementation of Alkylglycerol on Inflammatory Markers

In this study, the high sensitivity C-reactive protein (hsCRP) was decreased significantly by 28%. CRP is an acute phase reactant triggered by the activation of cytokines. It is regarded as prothrombotic and proatherogenic in nature, and is commonly used as marker of systemic inflammation. This suggests that the supplementation of alkylglycerol reduces systemic inflammation in humans.

### Conclusion

The supplementation of alkylglycerol (in the form of shark liver oil) modulates plasmalogens in human plasma, in terms of absolute concentrations, levels relative to lipoprotein content and in alkenyl chain composition. These modulations were each associated with a reduction in obesity-related dyslipidemia (a decrease in total cholesterol and triglycerides). A decreased white blood cell count was observed, due primarily to a reduction in the number of neutrophils. Furthermore, the levels of intermediate monocytes were decreased following alkylglycerol supplementation, as was the level of hsCRP, a measure of chronic inflammation. Overall, SLO supplementation tended to have a beneficial effect on multiple readouts linked to obesity and metabolic syndrome.

### EXAMPLE 3

### Modulation of Plasmalogens by Alkylglycerol Supplementation in Mammals

A supplementation study was conducted in which mice were fed different supplementation diets over 20 weeks, with lipids quantified in different organs (plasma, adipose, heart, liver, and skeletal muscle) at the end of this period. Diets included normal chow, high fat diet (HFD), HFD with an alkylglycerol (AKG) mix, and HFD with three increasing quantities of SLO.

After 20 weeks of supplementation, mice were euthanised and blood and tissues (liver, adipose, heart and skeletal muscle) were collected. Plasma was separated from blood by centrifugation. The different PE plasmalogen species of plasma and different tissues were then measured by targeted lipidomics.

In this example, six-week old male C57BL/6J mice housed at 6 mice per cage at 22±1oC on a 12:12 h light/dark cycle were provided with ad libitum access to either a standard chow diet or a high fat diet supplemented with shark liver oil (SLO) or an alkylglycerol mix as follows (n=12 per group) for 20 weeks:
“CD” Group: mice fed with a chow diet only.
“HFD” Group: mice fed with a high fat diet (HFD) (43% energy from fat) only.
“HFD+AKG” Group: mice fed with a HFD containing 0.625% mixture of the three alkylglycerols (batyl alcohol, chimyl alcohol and selachyl alcohol) (1:1:1).
“HFD+0.25% SLO” Group: mice fed with a HFD containing 0.25% SLO.
“HFD+0.75% SLO” Group: mice fed with a HFD containing 0.75% SLO.
“HFD+1.88% SLO” Group: mice fed with a HFD containing 1.88% SLO.

After 20 weeks of supplementation, mice were euthanised and blood and tissues (liver, adipose, heart and skeletal muscle) were collected. Plasma was separated from blood by centrifugation. The different PE plasmalogen species of plasma and different tissues were then measured by targeted lipidomics.

### Results

Different Organs have Different Basal Compositions

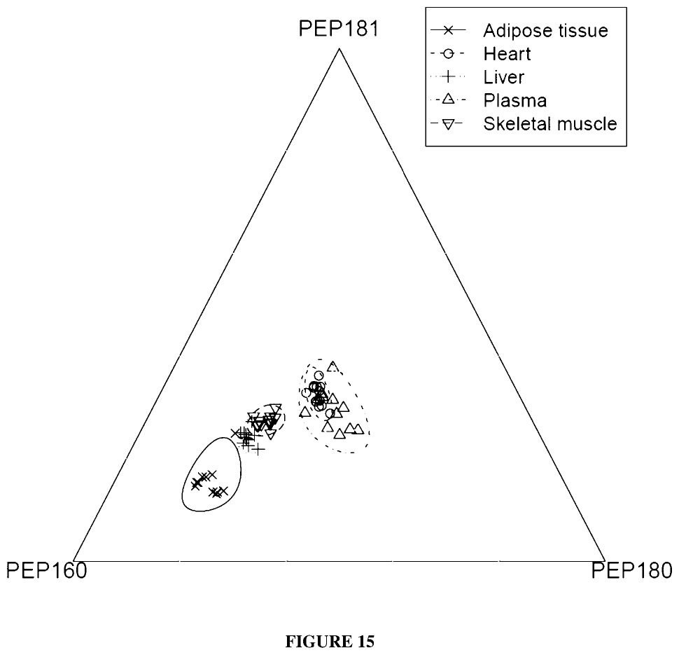

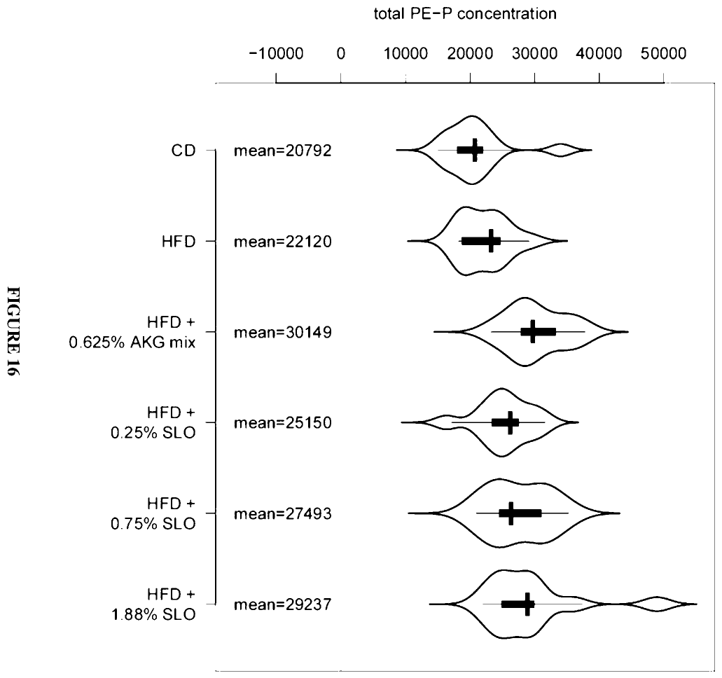

We can first visualise the alkenyl compositions of various tissues in mice on the typical chow diet. As illustrated in FIG. 15) the different organs have sometimes markedly different compositions, presenting what seems like an increasing gradient of O-16:0 from plasma & heart through skeletal muscle & liver up to adipose tissue.

### Supplementation Increases Total Plasma PE(P) Levels in Mammals

Similar to Example 2, total plasma PE(P) levels are increased by the various diets, in particular in AKG and SLO supplementation (FIG. 16). AKG significantly increased total PE(P) levels (estimate=+4479 pmol/mL; p-value<0.05), and so does SLO (estimate=+3322 pmol/mL per 1% of SLO; p-value<0.05) (based on a linear model, adjusted R squared 0.292).

Different Supplementations have Different Compositional Effects

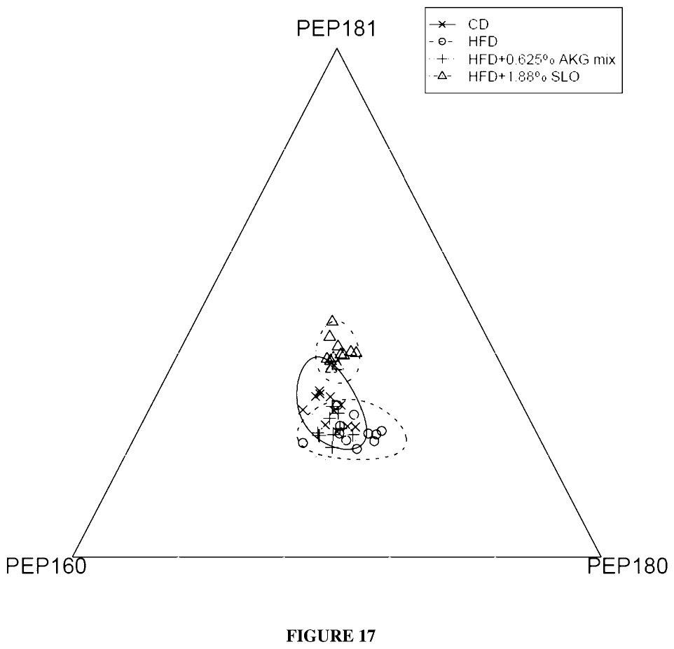

A comparison of the effects of different diets on plasma PE(P) alkenyl composition is illustrated in FIG. 17. In plasma, the HFD skewed the PE(P) alkenyl composition towards lower O-18:1, while SLO (high concentration) skewed it towards high O-18:1. The AKG seemingly stabilised the composition (lower variability than HFD).

### Response to Supplementation is Dose-Dependent

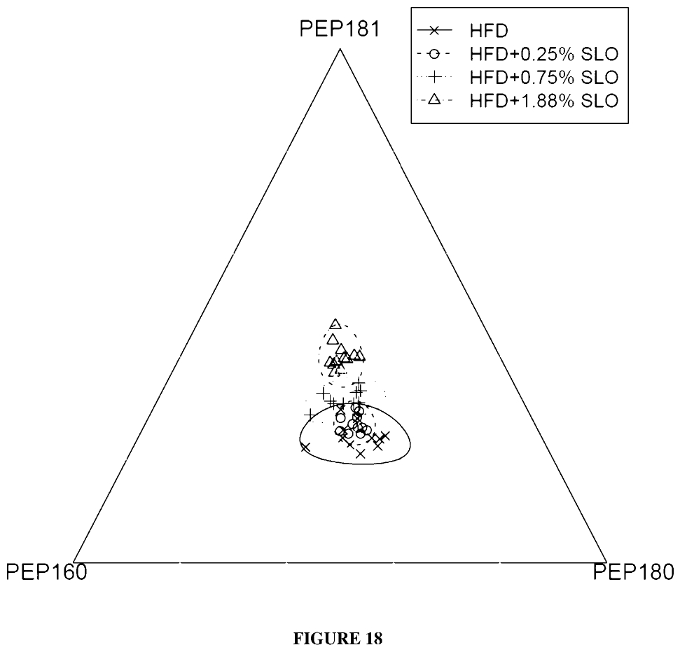

A comparison of the effects of different levels of SLO supplementation is illustrated in FIG. 18. The response is dose-dependent: higher SLO concentrations lead (unsurprisingly) to higher O-18:1 levels. It can be noticed that the mice given mid-range SLO concentration (0.75%) have a plasma composition (33% O-16:0; 35% O-18:0; 32% O-18:1) that is quite close to that of the chow diet (36% O-16:0; 34% O-18:0; 29% O-18:1; see FIG. 17 suggesting that this level of supplementation may counteract the compositional effect of the HFD.

### Response to Supplementation is Organ-Dependent

Different organs have different baseline (chow diet) compositions. They also have different responses to the supplementation (see FIG. 18):

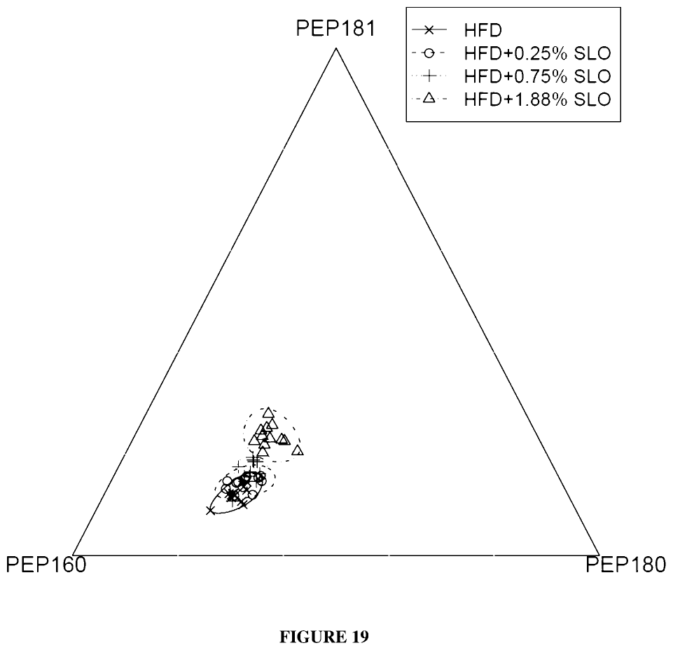

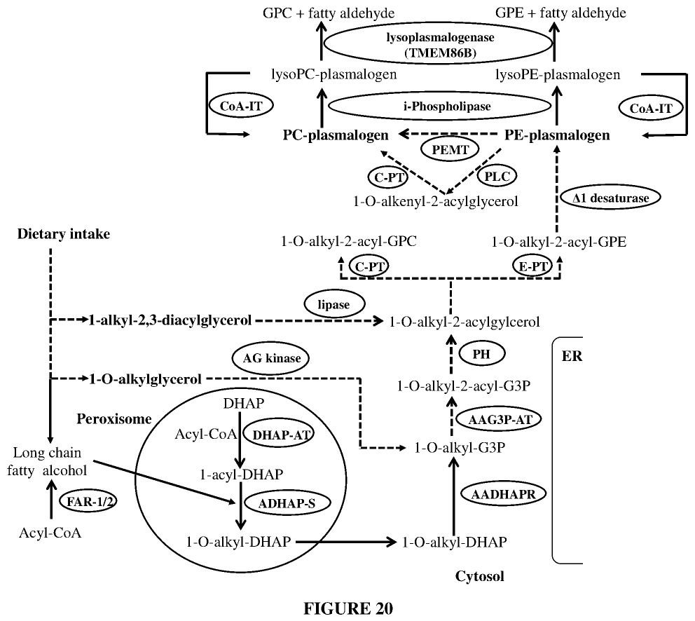

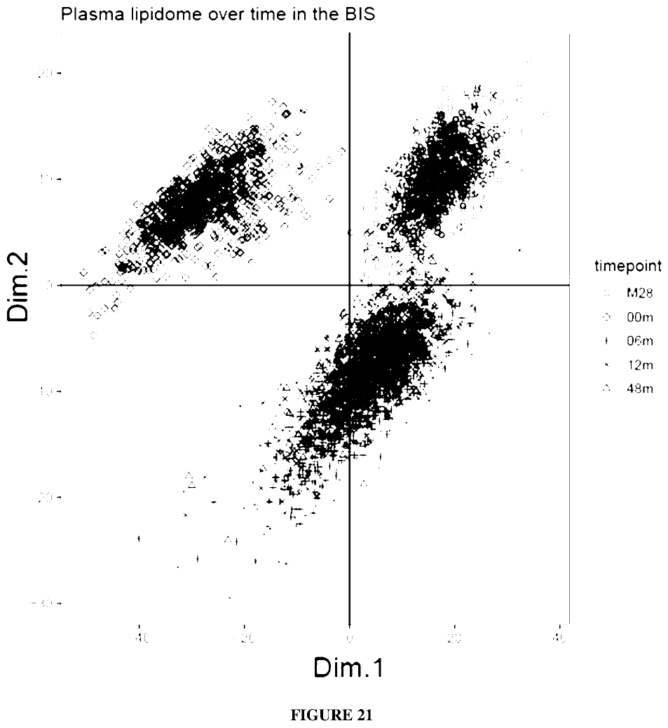

As shown in FIG. 18, increasing levels of SLO supplementation increased the 18:1 part in plasma (roughly 25% to 40%), concomitantly reducing the O-16:0 and O-18:0 parts (35% to 30% and 40% to 30%). FIG. 19, on the other hand, shows that increasing levels of SLO supplementation have a different effect on adipose tissue: O-18:1 is still increased (12% to 23%), however the O-18:0 part is maintained (at about 25-26%) with only O-16:0 being decreased (63% to 51%).

### Conclusion

As determined herein, the mouse was a good model of PE(P) modulation. Indeed, the effects of SLO (and to a certain extend AKG) supplementation pheno-copied those obtained in human SLO supplementation in Example 2: Overall, plasma PE(P) levels were increased following supplementation and alkenyl composition was skewed towards O-18:1.

This study also afforded the opportunity to explore various supplementation schemes and their effects on multiple organs. Overall, it is apparent that in mammals, PE(P) alkenyl composition differs from organ to organ, and that different diets & supplementation compositions can influence the PE(P) composition in different ways (AKG and SLO supplementation had different effects), with mid-concentration SLO being able to revert the HFD composition towards to that of basic chow in plasma. Furthermore, dose-dependent effects vary from organ to organ.

Accordingly, plasmalogen modulation therapy can be crafted to maintain homeostatic plasmalogen compositions.

Within the three examples provided are several important new findings with regard to plasmalogen modulation therapy:
1) Plasmalogen PE(P) alkenyl chain composition is under tight regulatory control.
2) Plasmalogen PE(P) acyl chain composition is similarly under regulatory control but this is less rigid.
3) The composition of plasmalogen alkenyl and acyl chains is tissue specific.
4) Other plasmalogen subclasses such and phosphocholine plasmalogens (PC(P) and other ether lipid subclasses such as PC(O), PE(O) LPC(O) will also be under similar control.
5) High fat diets or metabolic disease can lead to alterations of the plasmalogen composition.
6) Supplementation with ether lipids as described herein such as alkylglycerol or 1-alkyl, 2,3-diacylglycerol can alter the plasmalogen alkenyl chain composition dependant on the formulation of the supplementation.

The tight control of PE(P) alkenyl composition in humans, alongside the celerity (3 weeks) of the washout after a moderate perturbation (SLO supplementation), indicates that there is a biological impetus to the observed ether lipid/plasmalogen homeostasis and ratios. The control of the acyl chains, although less stringent, is under biological control. Dietary supplementation can impact this controlled balance, and though no adverse effects of reasonable supplementation have been reported so far, it is contemplated that supplementation with a lipid mixture composition that maintains or restores “healthy” or “non-disease” ether lipid molecule levels or rations such as plasmalogen levels and alkenyl compositions would provide less of a metabolic challenge to the recipient organism leading to a more efficacious treatment.

### EXAMPLE 4

### Supplementation to Modulate or Maintain Healthy Ether Lipid Molecule Levels and/or Ratios in Breast Milk

The ratio and or levels of ether lipid molecules in human breast milk is determined in a population of healthy mothers following the methods in the description and Example 1. In one embodiment a composition is contemplated as a nutritional supplement for women intending to breast feed or for breast feeding women comprising a mixture of ether lipid molecules for in vivo maintenance of ether lipids at levels and/or ratios associated with a non-disease state, or wherein the composition is for in vivo modification of ether lipids towards levels and/or ratios associated with a non-disease state. In one embodiment, the composition may be the active in a nutritional supplement. In one embodiment, the compositions may be used in therapeutic, prophylactic and maintenance administrations or a period of time and under conditions suitable for maintaining or modifying the ether lipid molecule composition of breast milk in a subject. In one embodiment, there is provided a composition which is a supplement for addition to infant formula milk comprising ether lipids, or a composition which is an infant formula milk composition comprising the ether lipids.

### Introduction

It is estimated that 20% of the world's adult population will be obese by 2030. The incidence of obesity among infants and children has increased by 30% globally in the last two decades. According to the Australian Bureau of Statistics' National Health Survey, in 2017-18, 67% of Australian adults and 24% of children are either overweight or obese [Alshehry et al, 2015]. Childhood adiposity is a well-established risk factor for future obesity and metabolic dysfunction in adulthood [Geserick et al, 2018; Hidayat et al, 2018]. Early childhood is also a period of high plasticity; hence understanding the factors contributing to adiposity and peripheral fat distribution in early life may be useful in early interception of the obesity and type 2 diabetes (T2D) epidemic. Longitudinal data indicate that obese children who normalise their weight before adulthood have metabolic and cardiovascular risks identical to those who are never obese [Juonala et al, 2011].

One in every nine Australians have asthma and wheezing illnesses are the most common cause of hospital admission in preschool aged children. In 2017-18 there were 40,000 hospitalizations with asthma, and 45% of them were children under the age of 14. Asthma is one of the top ten burdensome diseases for children up to 15 years. From 2010-2014, the mortality rate for asthma among Aboriginal and Tones Strait Islanders was twice that of non-Aboriginal Australians [Alshehry et al, 2015]. The high prevalence of childhood asthma could be tackled with improved understanding of the risk factors predisposing individuals to the development of asthma.

Breastfeeding protects infants from developing obesity and asthma. However, the 20^{th} century witnessed an increase in formula feeding [Riordan et al, 1980]. Currently, only 35% of infants are exclusively breastfed in the first six months of their life [Juonala et al, 2011]. In the past 40 years, several studies have reported the link between breastfeeding and lower risk of childhood obesity [Owen et al, 2005; Uwaezuoke et al, 2017]. Breast milk is rich in immunological components that are helpful in the development of innate and adaptive immunity. Breastfeeding is shown to lower the rates of wheezing in the first year of life and also lower the risk of asthma in the first three years of life. In a meta-analyses of 117 studies, Dogaru et al. identified a 22% reduced risk of asthma before two-years of age with prolonged breastfeeding [Dogaru et al, 2014]. Breastfeeding has other advantages such as lowering the risk of respiratory and other infections, sudden infant death syndrome, and T2D in the mother and the infant later in life [Mayer-Davis et al, 2006]. Despite the growing evidence for a relationship between breastfeeding and health outcomes, the underlying mechanism(s) remain poorly defined.

The primary goal of this study was to identify key components in breast milk that protect against obesity and other adverse growth and developmental outcomes. We have established evidence to support the development of nutritional supplements that could be incorporated into infant formula to afford the same protection as breastfeeding, and thereby reduce the levels of childhood obesity and other adverse growth and developmental outcomes.

Dysregulation of lipid metabolism is recognized as a primary driver of obesity and more recently of inflammation and immune regulation [Paul et al, 2019]. Breast milk is composed of about 3% fats (lipids). Roszer et al. recently reported that alkylglycerol-type (AG) ether lipids in breast milk maintain beige adipose tissue (BeAT) in infants and delay the transformation of BeAT into white adipose tissue in mice, thereby protecting against obesity. They further report that breast milk AGs are metabolized by adipose tissue macrophages to platelet-activating factor (PAF), which ultimately activates IL-6/STAT3 signaling in adipocytes and triggers BeAT development in the infant. This study suggests that lack of AG intake in infancy leads to premature loss of beige adipose tissue and increased fat accumulation and points to a role in immune cells in this process [Yu et al, 2019].

Alkylglycerols can also be metabolized into ether phospholipids including plasmalogens. We, and others, have identified a class of lipids (plasmalogens) that are critical for human health and are depleted in metabolic disease. Plasmalogens function in oxidative stress, inflammation, cholesterol metabolism and efflux, and cell signaling [ Paul et al, 2019]. Plasmalogens are decreased in obese individuals [Huynh et al, 2019] and are negatively associated with cardiometabolic disease, including diabetes and heart disease [Paul et al, 2019].

We have found that plasmalogens are amenable to modulation by dietary intervention with naturally occurring precursor compounds called alkyl/alkenyl glycerols.

From our lipidomic analysis of the infant plasma samples of the Barwon Infant Study (BIS), we have observed a dramatic difference in plasma lipids between breast fed and formula fed infants. Of particular note, species of alkyl-/alkenyl- diacylglycerol (the major form of alkyl-/alkenyl- glycerol in breast milk) and plasmalogens were markedly elevated in infants who were breast fed compared to those who were not. These findings provide clear evidence that breastfeeding has a major effect of lipid metabolism in the first year of life.

In this project, we aimed to compare the lipid composition, particularly ether lipids, between breast milk, animal milk and formula and to identify the key lipids that are driving the dramatic difference in plasma lipids between breast fed and formula fed infants.

### EXAMPLE 4A

### Plasma Lipidomic Analysis of 6-Month Old Infants in the Barwon Infant Study

### Methods

The Barwon Infant Study: The Barwon Infant Study (BIS) was designed and funded to investigate how environmental, genetic and epigenetic factors interact to influence the development of allergy and respiratory function, cardiovascular development and atherosclerosis and food allergy, the microbiome and neurodevelopment. Cohort entry was recruited using an unselected antenatal sampling frame. Women were recruited prior to 32 weeks of pregnancy between June 2010 and June 2013 within the Barwon Health region. Exclusion criteria were: (a) severe congenital heart disease; (b) multiple congenital anomalies; (c) any situation where it is felt inappropriate to seek consent in the opinion of the attending nurse or midwife; (d) home delivery; and (e) delivery prior to 35 weeks. 1155 families were enrolled antenatally, providing 1074 eventual eligible live-born infants. The 4-year comprehensive review was completed in 2017.

Comprehensive questionnaire data and clinical measures are available antenatally and at birth, 4 weeks, 3, 6, 9 and 12 months and 2 and 4 years. Data includes parental health and demographic, antenatal and postnatal lifestyle and medical history, maternal antenatal diet, medication and supplementation use, mental and other health history, perinatal characteristics, breast feeding infant illness, diet, food reactions, medications and supplements, health resource utilisation, child lifestyle, including physical activity, sun exposure, time outside and TV and other screen time. Clinical measures include serial growth and adiposity measures, skin type and a range of detailed phenotype indicators of system development and disease that are listed below. Prenatal maternal blood (28 weeks); cord blood, placenta and meconium at birth; infant blood, urine, faeces and hair art regular intervals (blood at 6, 12 and 48 month) were collected. Monocyte pellets were isolated from the whole blood [Vuillermin et al, 2015]. Breast milk was collected from 295 participants when the infants were 1 month old and from 33 participants when the infants were 6 and 12 months of age. Data on asthma incidence has been collected at the preschool review (4.2±0.3 years of age, mean±SD) in 895 children, with 143 (16%) classified with doctor diagnosed asthma [Gray et al, 2019; Gray et al, 2019].

### Lipidomic Analysis:

Lipidomic analysis of the plasma samples from the six month old infants was performed as described above.

### Statistical Analysis:

Lipid-outcome association analyses: Using BIS lipidomics and demographic data, we performed linear regressions modelling the impact of factors such as maternal BMI, gestational age, child gender and more on the infant lipidome at 06 m, 12 m and 48 m of age. The impact of each factor (as corrected by all other factors included in the linear models) could then be reported on for each lipid class/species, giving association strengths (typically percent differences between groups or per unit increase of the factor), 95% confidence intervals thereof, and significance levels (corrected for multiple testing). Such results can be represented in forest plots.

Lipid forest plot: Lipid forest plots in this document represent the strength (with 95% confidence interval) and statistical significance of the associations of individual lipid species (and/or class totals), all ordered by class and species along the y-axis, with a particular outcome (potentially controlling for additional covariates). The x-axis is generally a regression coefficient, percentage difference, fold change or similar that captures the strength of the association.

Principal Components Analysis: PCA is a multivariate analysis technique that seeks to find Principal Components that summarise/explain a maximum amount of variability in the entire dataset. A scree plot is typically used to determine the number of PCs of interest. Individual samples can then be represented on these components using score plots, and distances, groupings, gradients or outliers can be interpreted. PCA can thus be used (amongst other things) to identify the strongest signals affecting a dataset. In this document all PCAs were applied to log-transformed lipid concentration data.

Violin plots: Violin plots, in a way analogous to histograms or boxplots, represent the distribution of a quantitative variable, optionally split out across multiple experimental groups. Each violin plot includes: a point representing the mean (blue), a point representing the median (white), a box in grey representing the central 50% of values, with “whiskers” extending outwards up to 1.5 times the inter-quartile range, as in more traditional boxplots.

Bar plots: in this section, unless otherwise indicated, bar plots show the mean levels (concentrations or proportions) of certain lipid species or sidechains, with whiskers extending outwards for one standard deviation, optionally split out across multiple experimental groups.

Ternary diagrams. A 3-part composition can be represented as a point in a triangular ternary diagram. Each summit of the triangle corresponds to a composition of 100% of one of the parts. The opposing base thus corresponds to a composition of 0% for that part. In all PE-P compositions looked at here (alkenyl or acyl), there are 3 sidechains that are generally more abundant. We can thus represent the compositions for these 3 chains in ternary diagrams.

It should be noted that the sidechain proportions reported on in the bar plots will not align with those shown in the ternary diagrams, as the latter rescale the total to that of the 3 sidechains selected for the diagram. This is particularly visible for PE-P acyls as there are more non-trivial sidechains than for alkenyl side chains.

### Results

### The Plasma Lipidome in 6-Month Old Infants:

As evidenced by the principal components analysis performed on the BIS lipidomics data, shown in FIG. 21, the plasma lipidome evolves over life, with clear differences between maternal, cord and infant plasma samples.

### The Effect of Breast Feeding on the Plasma Lipidome of 6-Month Old Infants:

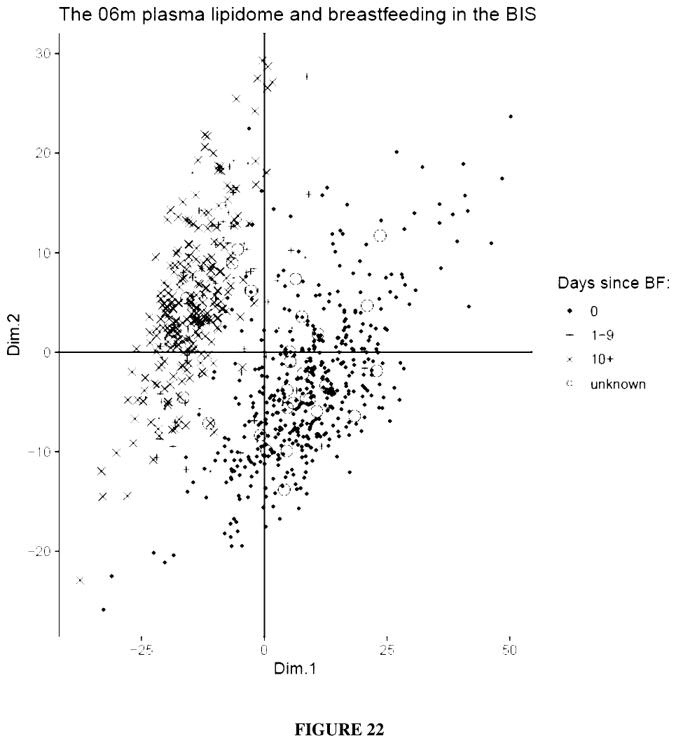

A PCA performed on the 6-month old infant plasma lipidomics samples (FIG. 22) shows the separation of recently versus non-recently breastfed infants across the first principal component, thus indicating that the strongest factor influencing the 6-month-old plasma lipidome is breastfeeding status.

### Breastfeeding and Plasma Lipids:

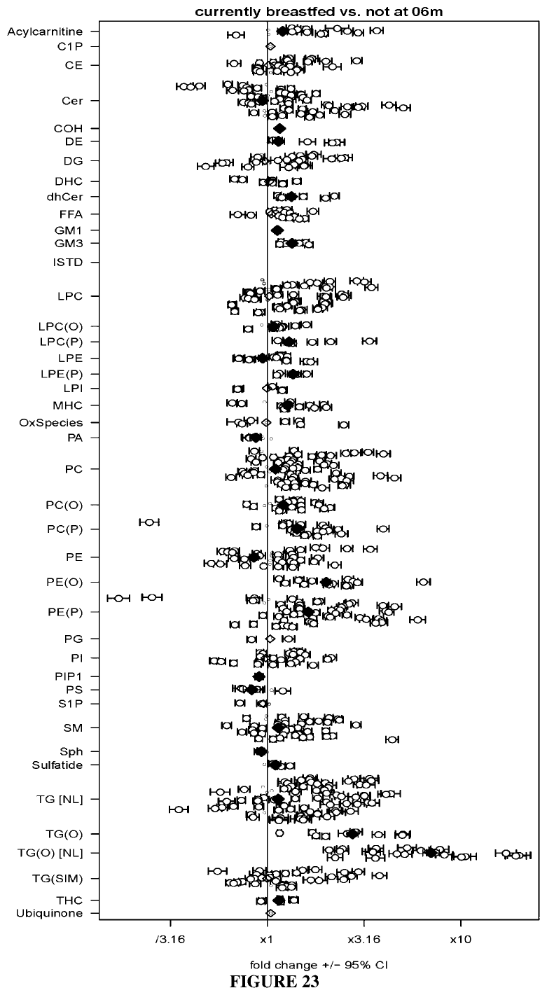

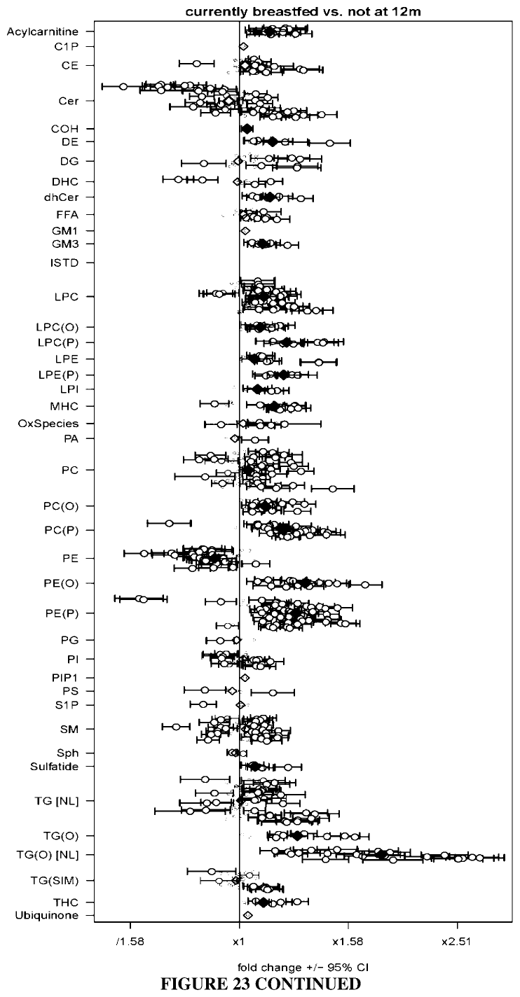

More than 600 lipid species were significantly associated with breastfeeding at both 6 and 12 months of age, after correcting for other factors such as gestational age and child gender, as well as for multiple testing (FIG. 23). Of particular note, species of alkyl-diacylglycerol and plasmalogens were markedly elevated in infants who were breast fed compared to those who were not. At a class level, these elevations were of the order 2-4 fold, while some individual species were elevated more than 17-fold. Overall, breastfeeding has a consistent and far-reaching impact on the concentrations of many of the lipids in the infant plasma lipidome.

### The Effect of Breast Feeding on PE(P) Plasmalogen Alkenyl and Acyl Sidechain Composition in 6-Month Old Infants:

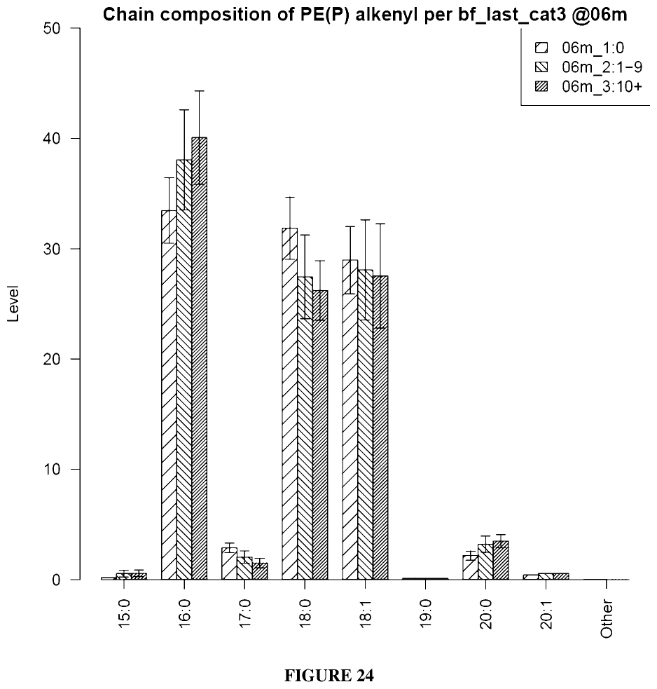

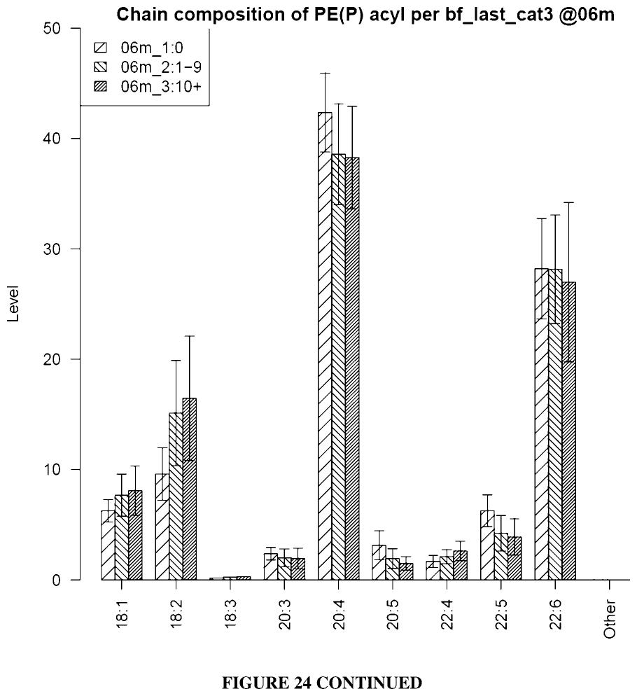

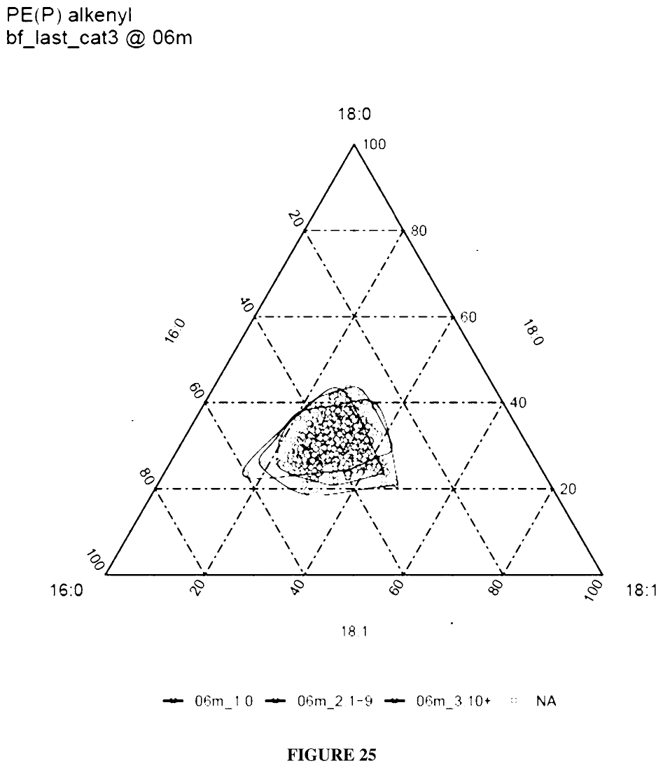

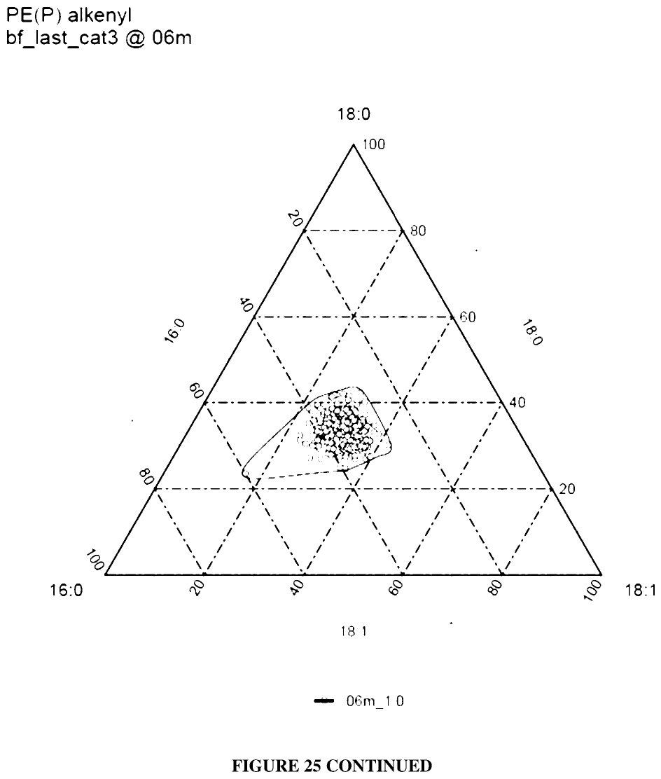

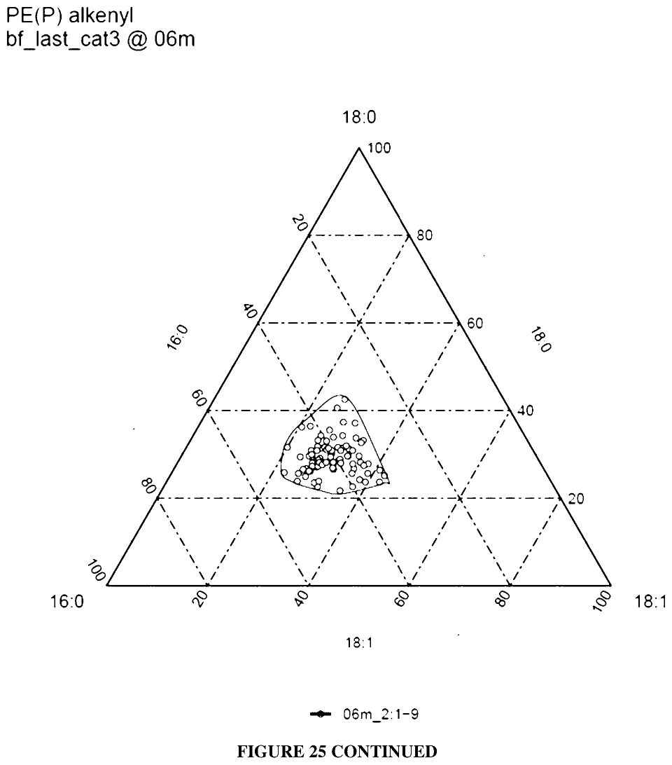

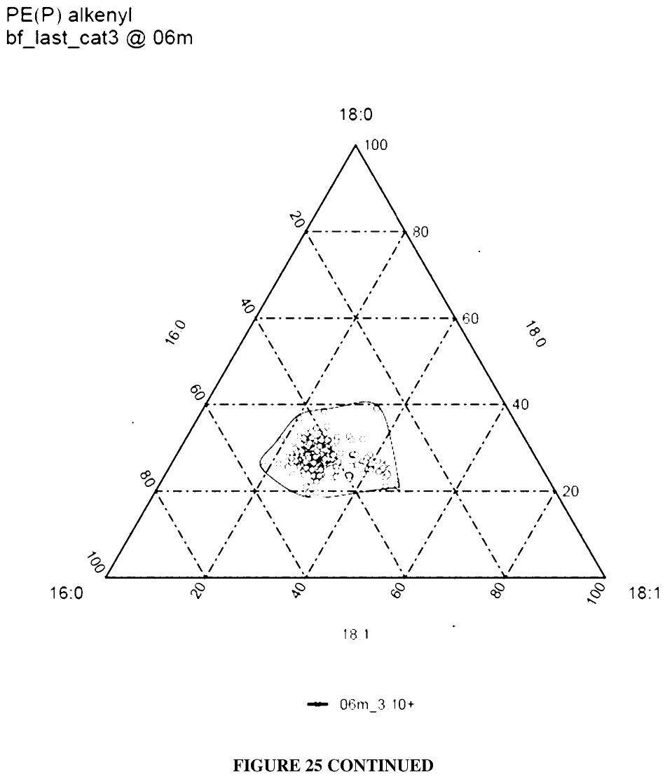

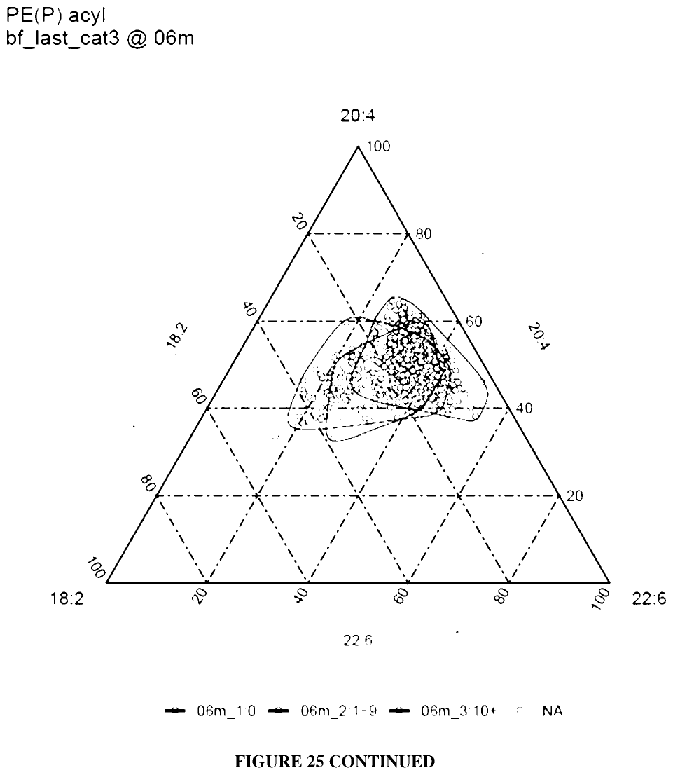

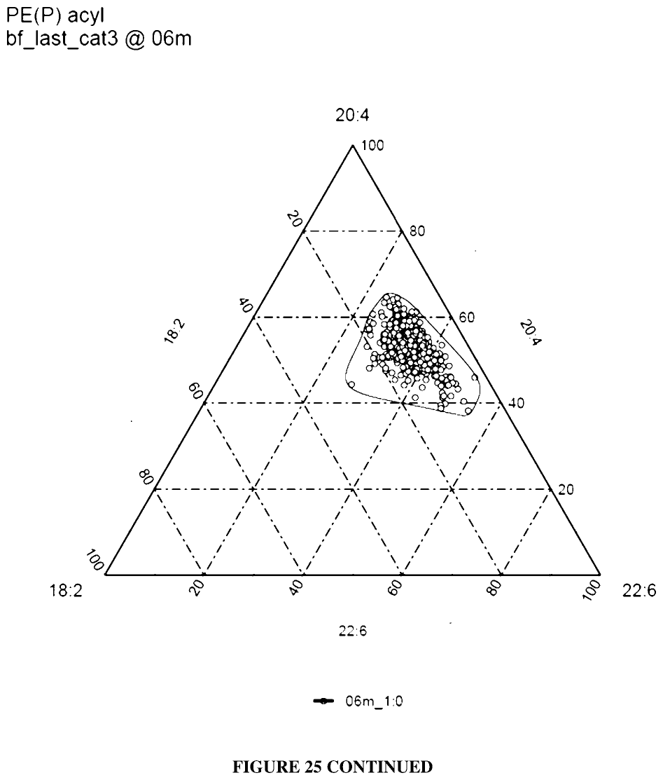

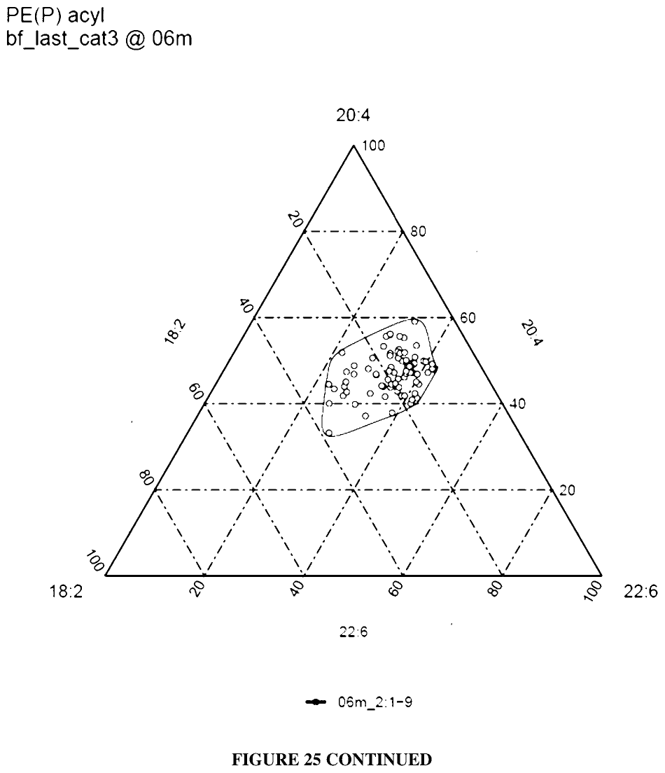

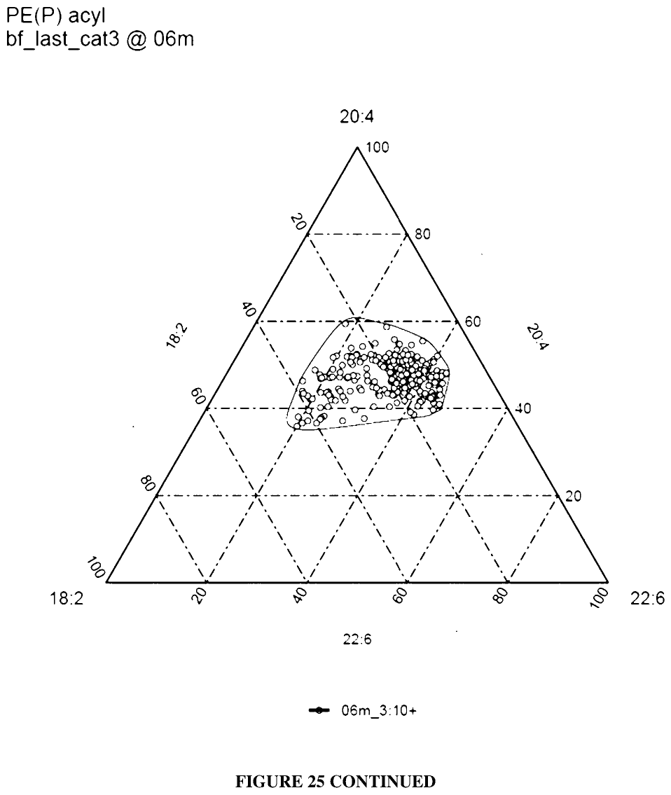

The bar plots in FIG. 24 show that PE(P) sidechain compositions change by breastfeeding status. For the most abundant alkenyl chains, there are lower proportions of 16:0 in recently breastfed (˜32%) and higher proportions in non-recently breastfed (˜40%), with concomitant decreases in the proportions of 18:0 (˜30% to ˜25%). For the most abundant acyl chains, 18:1 and 18:2 increase with time since last breastfeeding, while 20:4 and 22:6 decrease. These changes are highlighted in the ternary diagrams shown in FIG. 25. Breastfeeding thus has a direct impact not just on concentrations as shown above, but also on the sidechain composition of PE-P lipids in the infant lipidome.

### The Effect of Breast Feeding on Alkyl-Diacylglycerol (TG(O)) Composition in 6-Month Old Infants:

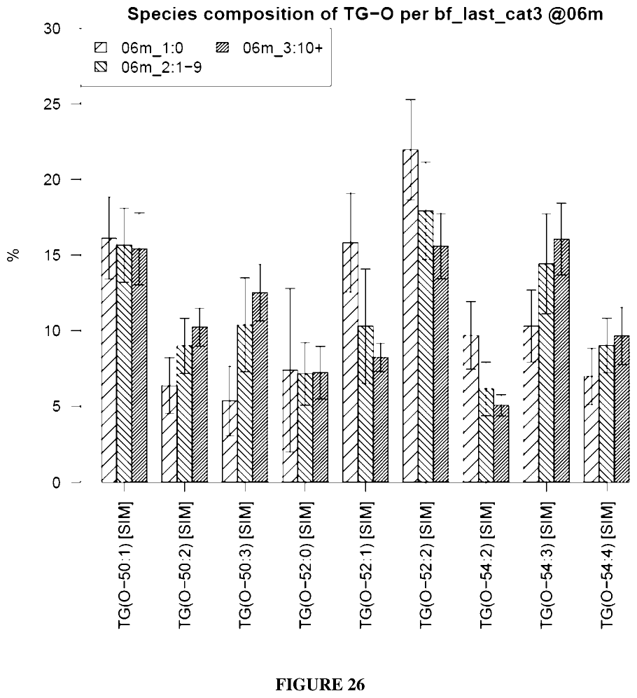

The bar plot in FIG. 26 shows that the average proportions of TG(O) species amongst total TG(O) change with respect to time since last breastfeeding. Indeed, species such as TG(O-50:1), TG(O-52:1), TG(O-52:2), and TG(O-54:2) decrease over time since last breastfeeding, while TG(O-50:2), TG(O-50:3), TG(O-54:3) and TG(O-54:4) increase. Breastfeeding thus has a direct impact on the species composition of TG(O)s on the infant lipidome.

### Summary and Conclusions

Breast feeding has a dramatic effect of the plasma lipidome of 6 month old infants. The effect is similar although somewhat attenuated in 12 month old infants, presumably as a result of the lower proportion that breast milk makes up of the diet. The higher levels of 18:2 FA in formula likely influence the composition of TG(O) in formula fed infants). These results enable development of a supplement to fortify infant formula with AG and or TG(O) species to raise the content of plasmalogens in infant plasma. Towards this end we provide the following composition of PE(P) alkenyl chains (Table 10) useful in the development of a suitable formula composition. This shows that the median % composition for P-16:0, P-18:0 and P-18:1 in the infants is 35.5%, 33.9%, and 30.7% respectively.

Furthermore, 50% of the infants have a composition in the following range P-16:0 (33.5%-37.4%); P-18:0 (31.8%-35.8%); P-18:1 (28.6%-32.5%). Thus a formulation that maintains the PE(P) alkenyl chain composition within these ranges (such that the sum total of P-16:0, P-18:0 and P-18:1 equals 100%) would be suitable for an infant formula supplement.

Finally, 90% of the infants have a composition in the following range P-16:0 (30.9%-40.7%); P-18:0 (28.8%-38.3%); P-18:1 (25.7%-35.8%).

TABLE 10
PE(P) alkenyl chain composition of 6-month infant plasma.

| Percentile | P-16:0 | P-18:0 | P-18:1 |
|---|---|---|---|
| 0% | 27.5 | 23.6 | 16.4 |
| 2.5% | 30.1 | 27.7 | 24.7 |
| 5% | 30.9 | 28.8 | 25.7 |
| 10% | 31.8 | 30.0 | 27.0 |
| 25% | 33.5 | 31.8 | 28.6 |
| 50% | 35.5 | 33.9 | 30.7 |
| 75% | 37.4 | 35.8 | 32.5 |
| 90% | 39.3 | 37.6 | 34.8 |
| 95% | 40.7 | 38.3 | 35.8 |
| 97.5% | 41.7 | 39.6 | 37.4 |
| 100% | 60.0 | 42.2 | 41.9 |

### EXAMPLE 4B

### Lipidomic Analysis of Breast Milk from Mothers in the Barwon Infant Study

### Methods

### The Barwon Infant Study (Breast Milk Samples)

A total of 313 breast milk samples from the mothers recruited into the BIS study, including samples collected from 247 participants when the infants were 1 month old and from 33 participants when the infants were 6 and 12 months of age, were analysed. Several animal milks (2 cow milk and 1 goat milk) and formula (n=10) were also analysed.

### Animal Milk and Infant Formula Milk Samples

Three animal milk products and 10 infant formula products (Table 11) were analysed for comparison to the human breast milk samples.

TABLE 11
Details of animal milk and formulae analysed in this study.

| ID | Description |
|---|---|
| MF 01 | Karicare ® Infant Formula (0-6 months) |
| MF 02 | Aptamil ® Gold Plus Premium Infant Formula (0-6 |
| | months) |
| MF 03 | Nestle ® NAN Comfort 1 Starter Infant Formula |
| MF 04 | S-26 ® Original Newborn Infant Formula (0-6 months) |
| MF 05 | Infacare ® SMA Infant Formula (0-12 months) |
| MF 06 | Bellamy's Organic Infant Formula (0-6 months) |
| MF 07 | Bubs ® Australian Goat Milk Formula (0-6 months) |
| MF 08 | Oli⁶ Goat Milk Infant Formula (0-6 months) |
| MF 09 | Karicare ® Soy Plant Based Formula |
| MF 10 | S-26 Gold ® Soy Infant Formula |
| Cowmilk 01 | Coles ® Full Cream Milk |
| Cowmilk 02 | Devondale ® Full Cream Milk |
| Goatmilk | Caprilac ® Goats Milk |

### Lipidomic Analysis:

Lipidomes of human breast milk, animal milk and milk formulae samples were analysed by the method described above (Lipidomic analysis). Two separate lipid extractions were performed with milk samples. Firstly, lipids were extracted from 10 μl of milk samples for the analysis of all lipid species except triacylglycerols. For the analysis of triacylglycerols, lipids were extracted from 10 μl of a 1 in 100 dilution of the milk samples (diluted with MiliQ water).

### Saponification of Milk Samples.

Lipids were extracted from 10 μl of breast milk, animal milk or formula samples using 100 μl of butanol and methanol (1:1) as described previously [Alshehry et al, 2015]. Following this, a portion of the lipid extract (80 μl) was dried under a constant stream of nitrogen. Then, 100 μl of 0.1 M sodium hydroxide in methanol was added to the dried extract and alkaline hydrolysis was carried out for 2 hours at 80° C. Following saponification, 10 μl of 1M formic acid was added to stop the hydrolysis reaction.

The hydrolysate was then dried under a constant stream of nitrogen and finally reconstituted with 200 μl butanol and methanol (1:1) (with 10 mM ammonium formate) containing a mixture of the internal standards. The extracts were mixed and stored at −80° C. until further analysis.

### Liquid Chromatography and Mass Spectrometry.

Standard lipidomic analysis was performed as described earlier (page 42 of original application; Lipidomic analysis) and analysis of saponified samples was performed as follows.

Analysis of lipid extracts was performed on an Agilent 6490 QQQ mass spectrometer with an Agilent 1290 series HPLC system and a ZORBAX eclipse plus C18 column (2.1×100 mm 1.8 μm, Agilent) with the thermostat set at 45° C. Alkylglycerol analysis was performed in the positive ion mode by applying characteristic multiple reaction monitoring (MRM) transitions while free fatty acid analysis was performed in negative ion mode by selected ion monitoring (SIM).

The solvent system consisted of solvent A) 50% H₂O/30% acetonitrile/20% isopropanol (v/v/v) containing 10 mM ammonium formate and solvent B) 1% H₂O/9% acetonitrile/90% isopropanol (v/v/v) containing 10 mM ammonium formate. The gradient was as follows; starting with a flow rate of 0.4 ml/min at 15% B and increasing to 50% B over 2.5 minutes, then to 57% over 0.1 minute, to 64% over 3.4 minutes, to 91% over 0.1 minute, to 97% over 2 minutes and finally to 100% over 0.1 minute. The solvent was then held at 100% B for 0.8 minutes (total 9 minutes). Equilibration was as follows: solvent was decreased from 100% B to 15% B over 0.1 minute and held for an additional 2 minutes (total cycle time 11.1 minutes).

The following mass spectrometer conditions were used: gas temperature, 150° C., gas flow rate 17 L/min, nebulizer 20psi, sheath gas temperature 200° C., capillary voltage 3500V and sheath gas flow 10 L/min.

For quantification of alkyl/alkenyl glycerol species, a deuterated monoacylglycerol (MG 18:1d7) was used as an internal standard. Response factors for alkyl-/alkenyl-glycerol species against MG 18:1d7 were calculated using serially diluted synthetic alkyl-/alkenyl-glycerol species in a range 1-300 μM and a fixed amount of MG 18:1d7. For quantification of free fatty acid species, deuterated free fatty acids were used as internal standards.

### Statistical Analysis:

PE-P sidechain composition. PE plasmalogens carry two side-chains, differing in their position and bond to the head group: an alkenyl chain and an acyl chain. Each can be of different carbon and desaturation numbers. We can sum up the total concentration of all lipid species carrying each type of chain and divide this by the total PE-P level to get the PE-P side-chain composition.

TG(O) and AG species composition. The composition of lipid species within a class can be expressed as a percentage of the ratio of each species concentration to the total for that class. In this example, we report on species compositions for the TG(O), and AG classes.

### Results

### The Breast Milk Lipidome

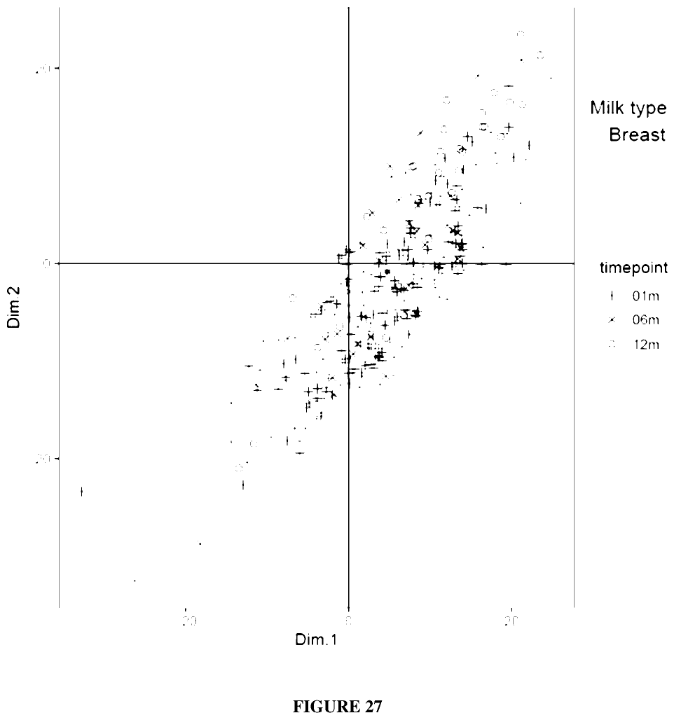

The PCA scores plot (FIG. 27) show the breast milk samples being spread across PCs 1 and 2, indicating a high level of sample-to-sample variability amongst these samples. This is not surprising, as breast milk composition is known to vary over infant age, time of day, breastfeeding schedule, maternal diet, and other factors. The average differences between breast milk taken at infant ages (1/6/12 months) are small, indicating that variations over infant age are less pronounced that those due to other sources of variation. Further examination (not shown) suggest that the later time points have slightly higher overall lipid levels, in particular for classes such as PC, PE, PC(P) and PS. Having established a high-level overview of variability in the breast milk lipidome, we looked to characterise the PE-P, TG(O) and AG composition of the breast milk.

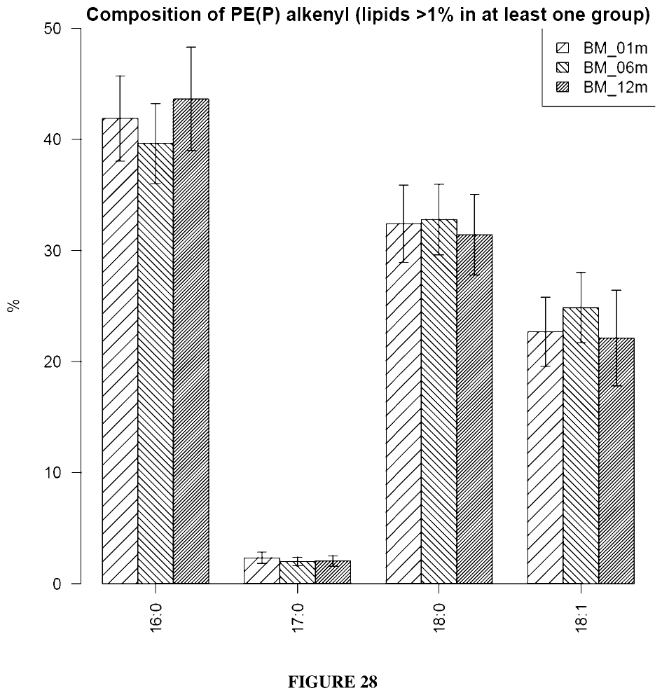

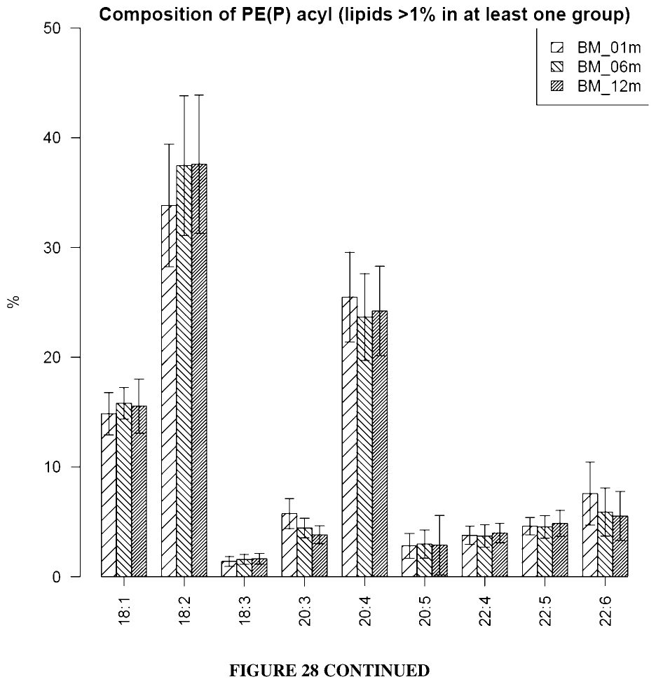

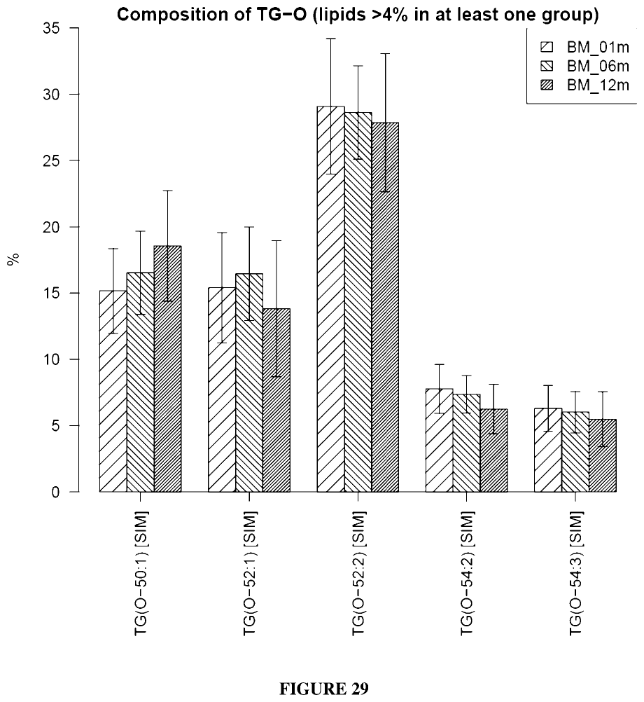

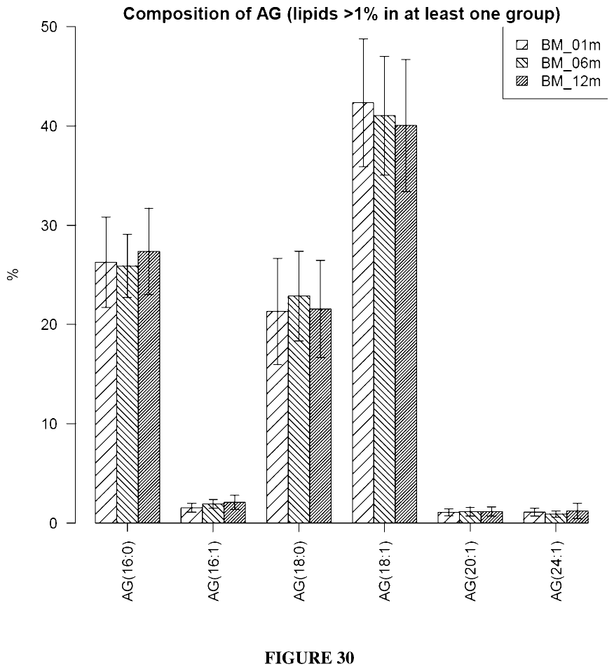

FIG. 28 shows the PE-P alkenyl and acyl chain composition of the breast milk samples, while FIG. 29 shows the TG(O) species composition for the same samples. FIG. 30 shows the alkylglycerol species composition. Differences between sampling ages (1, 6 or 12 months) are minor, similar to what was observed in the overall lipidome concentrations in the PCA above. Thus, the PE-P alkenyl and acyl chain composition and TG(O) and AG species compositions of breast milk are consistent across infant ages.

Comparison of the Breast Milk Lipidome with Animal Milk and Infant Formula Lipidome

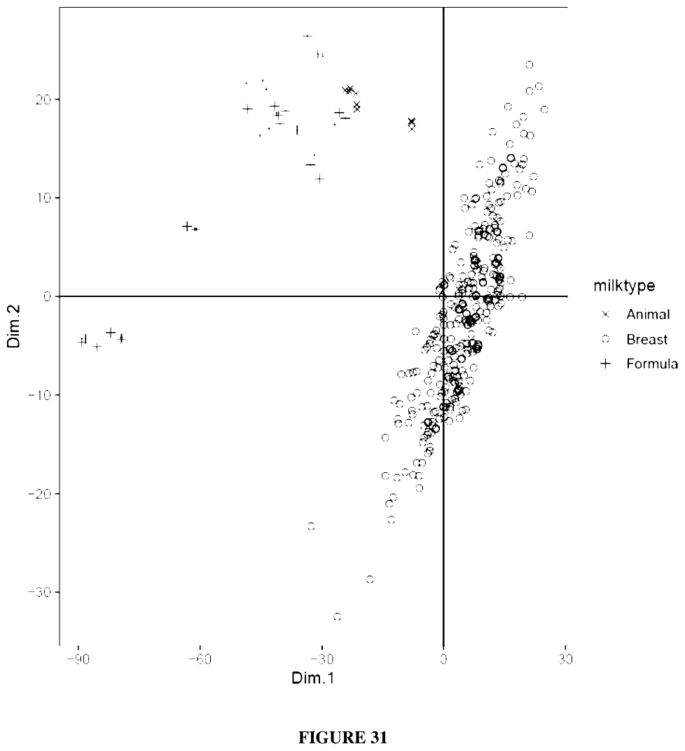

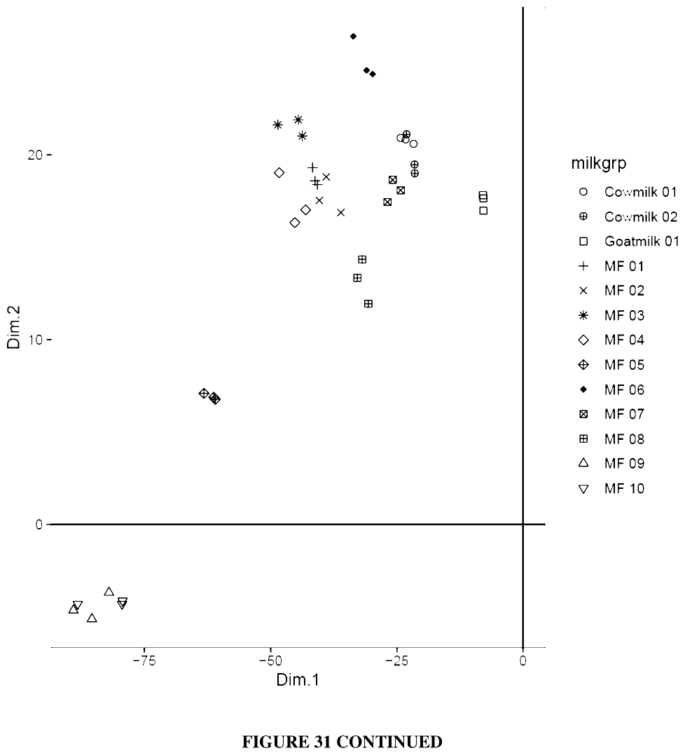

The PCA scores plots in FIG. 31 show how markedly different both animal (cow, goat) and formula lipidomes are from the breast milk lipidome and from each other. This clear distinction in lipidomes across different milk samples led us to compare the content and composition of PE(P), TG(O) and total AG between the milk types.

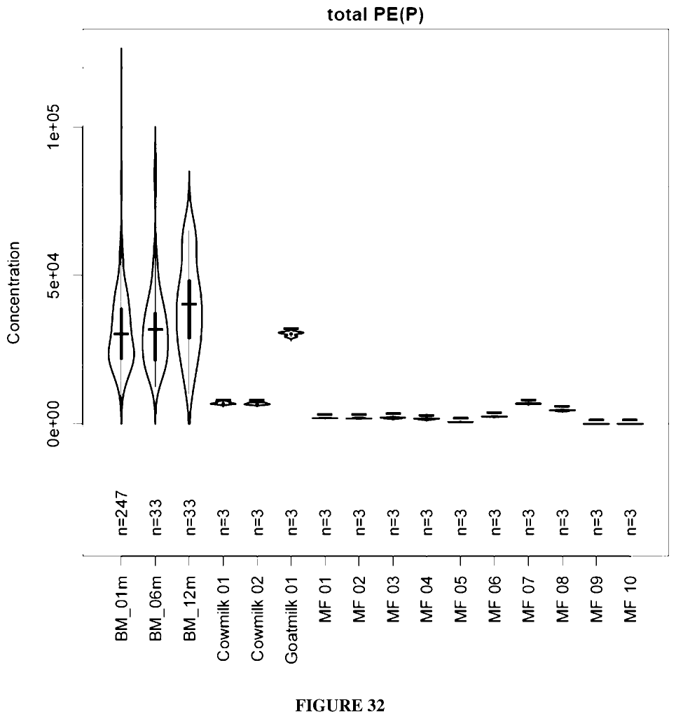

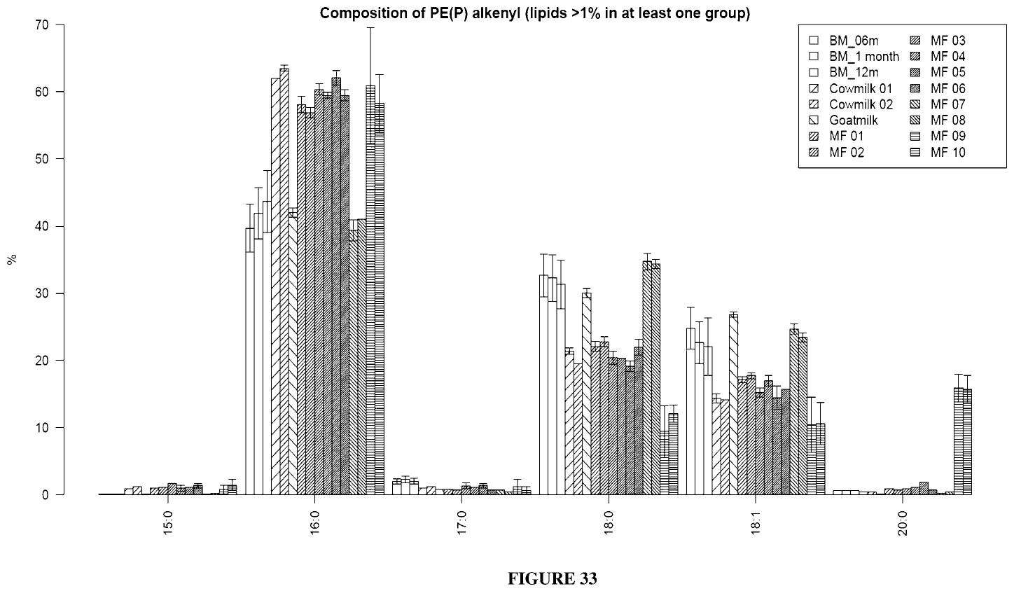

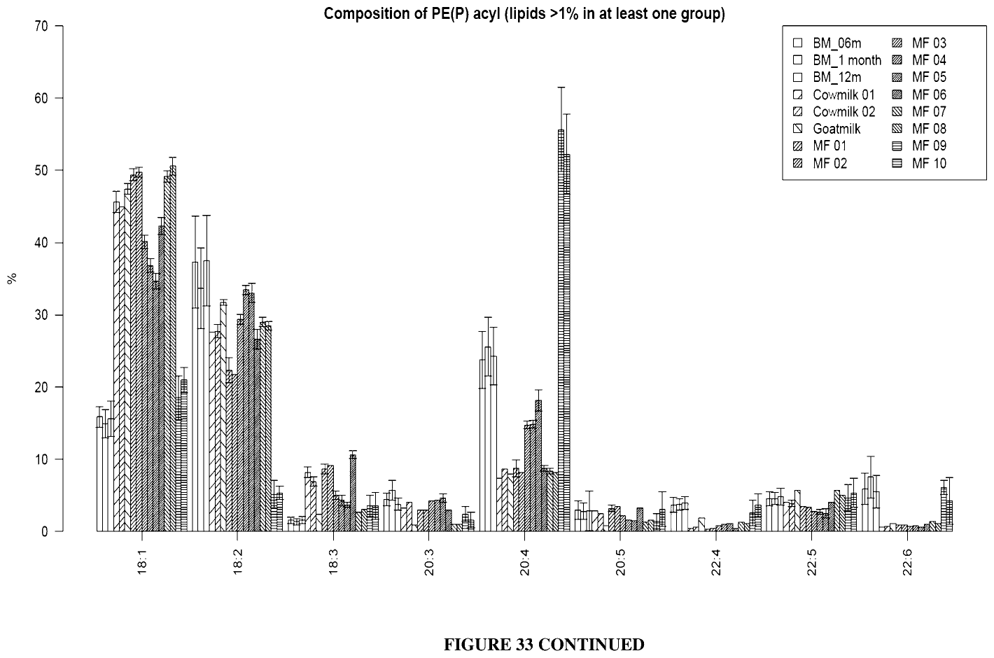

When we looked at the class level, we found that breast milk has much higher PE(P) content compared with animal milk or formula (FIG. 32). Further to that, we compared the alkenyl and acyl chain composition within PE(P) lipids across the milk samples. FIG. 33 shows the PE-P sidechain composition across all milks. For alkenyl side chains, goat milk samples typically are the closest to breast milk samples, while cow milks and cow milk-based formula show higher levels of 16:0 (˜60% versus 40%), lower levels of 18:0 (˜20% versus 30%) and 18:1 (15% versus 20%). Soy-based formula generally resembles cow milk formula, although with higher 20:0 (˜15% versus 0%) at the expense of 18:0 and 18:1. For acyl chains, the milks and formula were much more diverse, with notable differences being the much higher levels of 18:1 in animal milks and formula, and the very high level of 20:4 in soy-based formula.

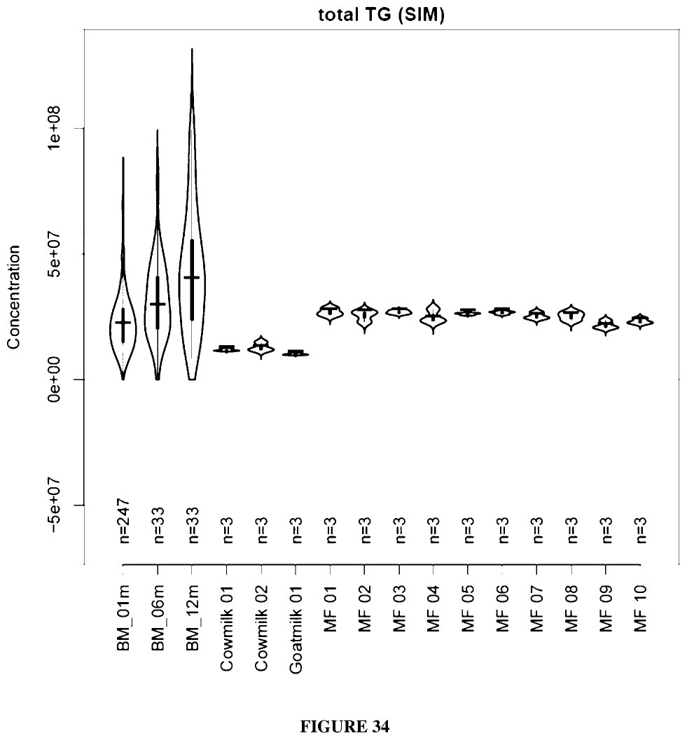

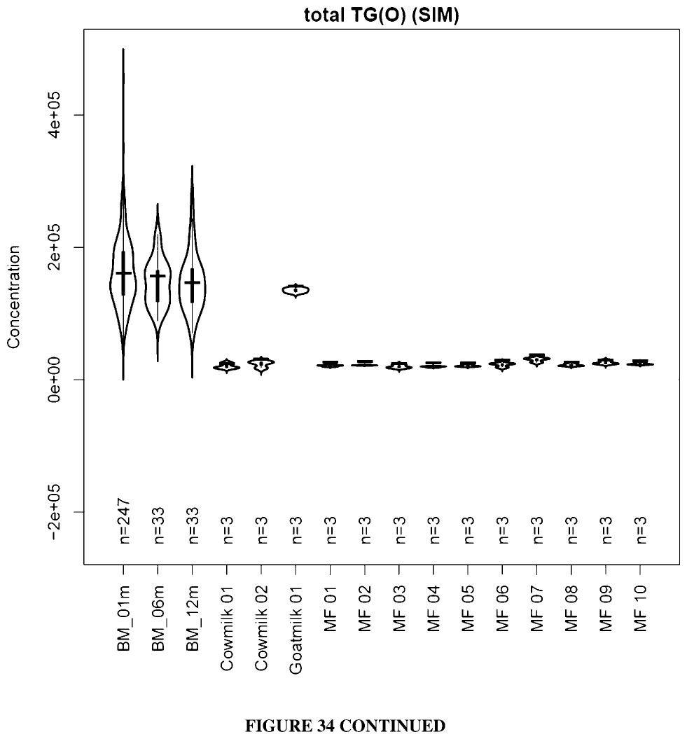

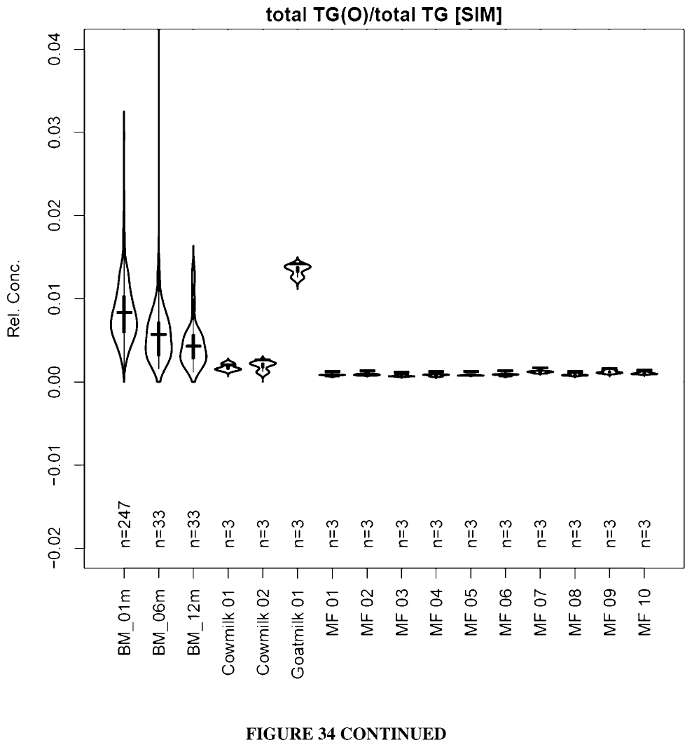

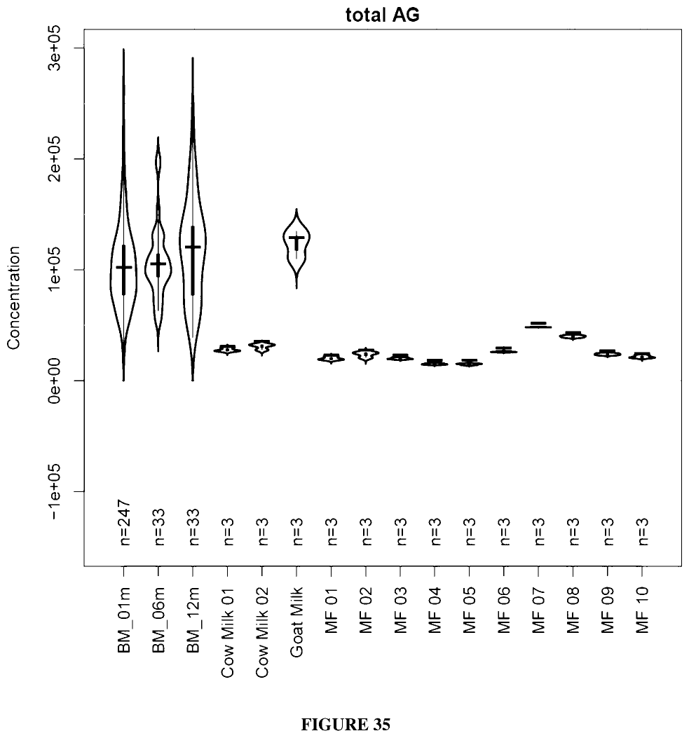

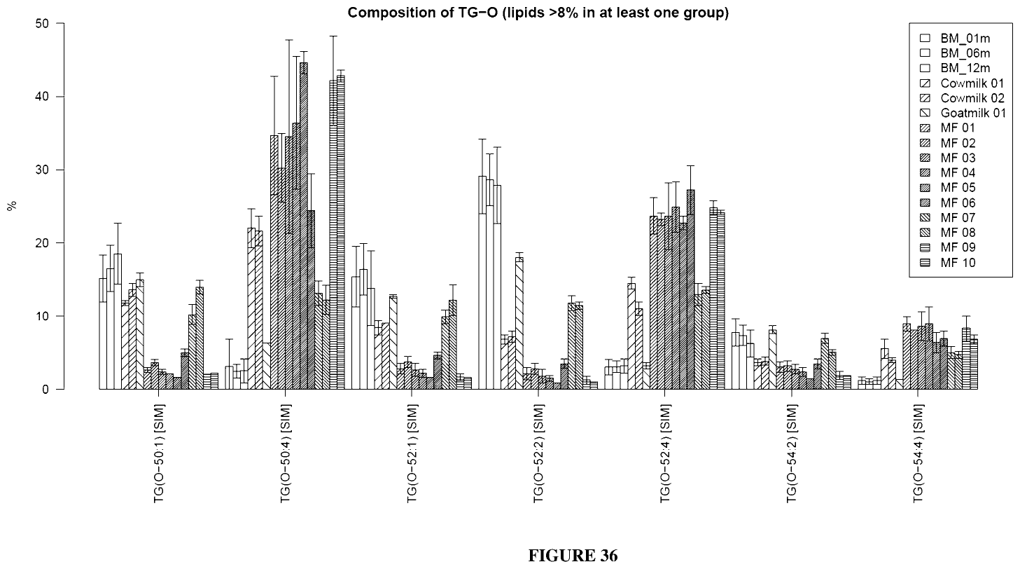

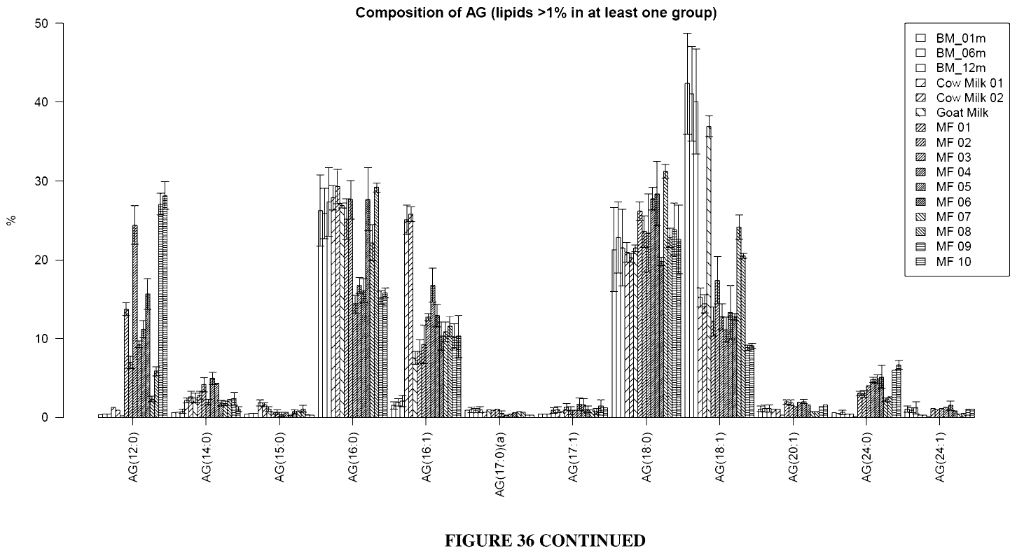

Breast milk also has higher content of TG(O) and total AG either as concentration or their relative proportion in total milk fat (FIGS. 34 & 35). We note that while TG content increases in breast milk with infant age (12 month>6 month>1 month) the TG(O) concentration was relatively stable across these ages. We further analysed the species composition of TG(O) and AG across the milk samples. FIG. 36 shows the TG(O) and AG species composition across all milks. None of the animal or formula milks recapitulate the TG(O) composition of the breast milk. The composition of major AG species in breast milk (26% AG(16:0), 22% AG(18:0) and 41% AG(18:1)) is stable across different time points but distinct from cow milks and formula. In particular, breast milk has higher proportion of AG(18:1) compared to cow milk or formula (41% versus 9-24%). Goat milk had the closest AG composition compared to breast milk.

Overall, it is clear that none of the animal nor formula milks manage to emulate the breast milk lipidome, either in terms of lipid concentrations (FIG. 31) nor compositions (FIGS. 33 & 36).

### Summary and Conclusions

Breast milk has a clearly distinct lipidome compared to animal milk and infant formula. In particular, breast milk has a stable but higher PE(P) and AG content compared to animal milk and formula. The alkenyl and acyl chain compositions of breast milk PE(P) are clearly different from animal milk and formula. Moreover, the AG composition of breast milk is distinct from all but goat milk and goat milk formula.

These results enable development of a supplement to fortify infant formula with AG and/or TG(O) species to raise the content and replicate the composition of human breast milk. Towards this end, we provide the following formulation table (Table 12). This shows that the median % composition for O-16:0, 0-18:0 and O-18:1 is 29:0%, 23.3%, and 47.1% respectively.

Furthermore, 50% of the mothers have a composition in the following range O-16:0 (26.2%-32.2%); O-18:0 (20.7%-25.5%); O-18:1 (43.3%-51.4%). Finally, 90% of the mothers have a composition in the following range O-16:0 (22.0%-37.4%); O-18:0 (18.2%-30.2%); O-18:1 (34.6%-56.9%).

TABLE 12
Alkylglycerol composition of human breast milk.

| percentile | AG(16:0) | AG(18:0) | AG(18:1) |
|---|---|---|---|
| 0% | 11.8 | 15.8 | 17.1 |
| 2.5% | 20.5 | 17.5 | 31.0 |
| 5% | 22.0 | 18.2 | 34.6 |
| 10% | 23.4 | 19.1 | 39.1 |
| 25% | 26.2 | 20.7 | 43.3 |
| 50% | 29.0 | 23.3 | 47.1 |
| 75% | 32.2 | 25.5 | 51.4 |
| 90% | 35.5 | 28.0 | 54.2 |
| 95% | 37.4 | 30.2 | 56.9 |
| 97.5% | 39.4 | 37.3 | 59.2 |
| 100% | 52.4 | 67.0 | 61.5 |

The composition of fatty acids in the TG(O) species that could be used in a supplement could include 16:0, 16:1, 18:0, 18:1, 18:2, 20:0, 20:1 to produce the major species TG(O-50:1), TG(O-52:1), TG(O-52:2), TG(O-54:2), TG(O-54:3) present in breast milk.

The amount of AG or TG(O) species added to such a supplement would be in the range that existing AG species are present in breast milk. Based on our analyses of 247 breast milk samples at one month of age the median concentration is 9904 with an inter-quartile range (25^{th} to 75^{th} centiles) of 78-122 μM and a 5^{th} to 95^{th} centile range of 53-176 μM.

Based on our analyses of 33 breast milk samples at six months of age the median concentration is 102 μM with an inter-quartile range (25^{th} to 75^{th} centiles) of 94-114 μM and a 5^{th} to 95^{th} centile range of 61-170 μM.

Based on our analyses of 33 breast milk samples at 12 months of age the median concentration is 117 μM with an inter-quartile range (25^{th} to 75^{th} centiles) of 77-139 μM and a 5^{th} to 95^{th} centile range of 59-190 μM.

Further ranges of alkenylphosphatidylethanolamine and the combined concentration of alkylglycerol and alkenylphosphatidylethanolamine are shown in Table 4.

TABLE 13
Alkylglycerol (AG) and alkenylphosphatidylethanolamine PE(P)
concentrations in human breast milk.

| | | | | | | | AG + | AG + | AG + |
|---|---|---|---|---|---|---|---|---|---|
| | AG | AG | AG | PE(P) | PE(P) | PE(P) | PE(P) | PE(P) | PE(P) |
| centile | (1M) | (6M) | (12M) | (1M) | (6M) | (12M) | (1M) | (6M) | (12M) |
| 0% | 21 | 54 | 39 | 5 | 12 | 10 | 26 | 69 | 49 |
| 2.5% | 42 | 56 | 55 | 13 | 17 | 14 | 56 | 71 | 71 |
| 5% | 53 | 61 | 59 | 14 | 18 | 17 | 70 | 78 | 78 |
| 10% | 60 | 71 | 61 | 18 | 18 | 19 | 76 | 90 | 83 |
| 25% | 78 | 94 | 77 | 22 | 21 | 29 | 101 | 120 | 105 |
| 50% | 99 | 102 | 117 | 29 | 30 | 39 | 129 | 130 | 162 |
| 75% | 122 | 114 | 139 | 39 | 38 | 48 | 162 | 165 | 185 |
| 90% | 153 | 134 | 178 | 47 | 45 | 62 | 198 | 184 | 225 |
| 95% | 176 | 170 | 190 | 55 | 54 | 64 | 225 | 232 | 254 |
| 97.5% | 200 | 196 | 199 | 61 | 62 | 65 | 274 | 234 | 263 |
| 100% | 269 | 199 | 231 | 115 | 84 | 65 | 374 | 238 | 295 |

Concentrations expressed as μmol/L. Green highlight shows the median values (50^{th} centile);
Yellow highlight shows the interquartile range (25^{th}-75^{th} centiles);
Blue shows the 10^{th} to 90^{th} centile.

### EXAMPLE 4C

### The Effect of Breast Feeding on Infant Growth Trajectories in the Barwon Infant Study

### Methods

### Lipidomic Analysis:

Lipidomic analysis of the plasma samples from the six month old infants was performed as described above.

### Statistical Analysis:

Calculation of growth trajectories. Growth trajectories have been constructed using birth, 1, 6, 12 and 48 month BMI measures, standardized according to the world health organization (WHO) guidelines. A latent class linear mixed model (LCLMM) was fitted to the centered zBMI measures with a shifted log transformed time scale, producing an optimal model with 3 classes.

Association of breast feeding with growth trajectories. Proportion breastfeeding at 6 months has been calculated for each of the growth trajectories. Pairwise p-values were determined using pooled two-proportions z-test, with a null hypothesis that the proportion breastfeeding is equal in both groups tested.

Association of plasma lipids with breast feeding. Linear regression of log transformed 6-month infant plasma concentrations against breastfeeding status at 6 months (breast fed vs formula fed) was performed adjusting for sex and weight. P-values were adjusted using the Benjamini-Hochberg procedure.

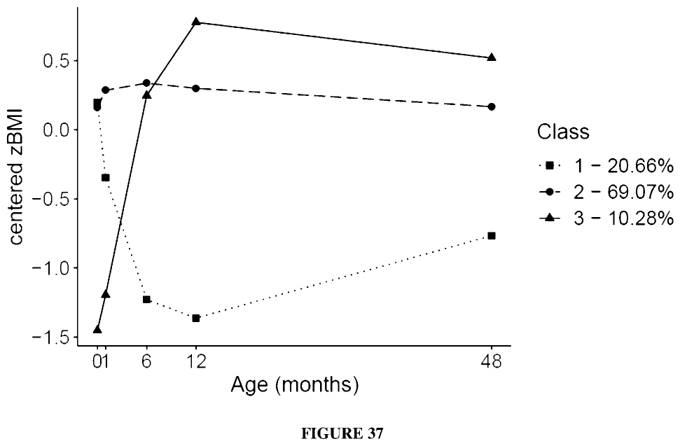

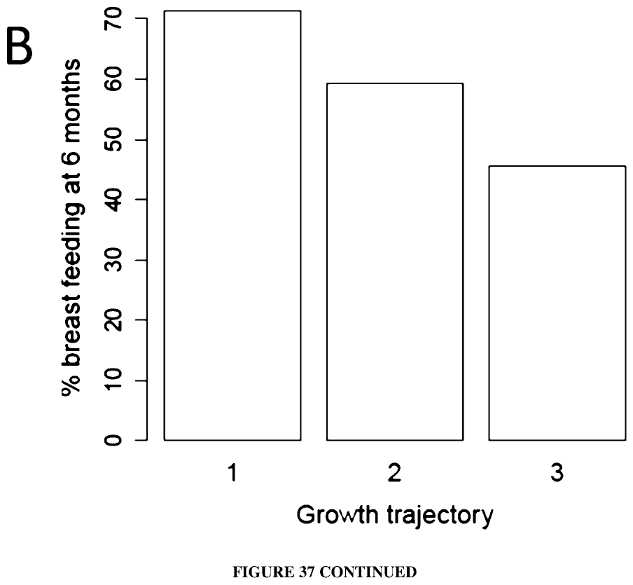

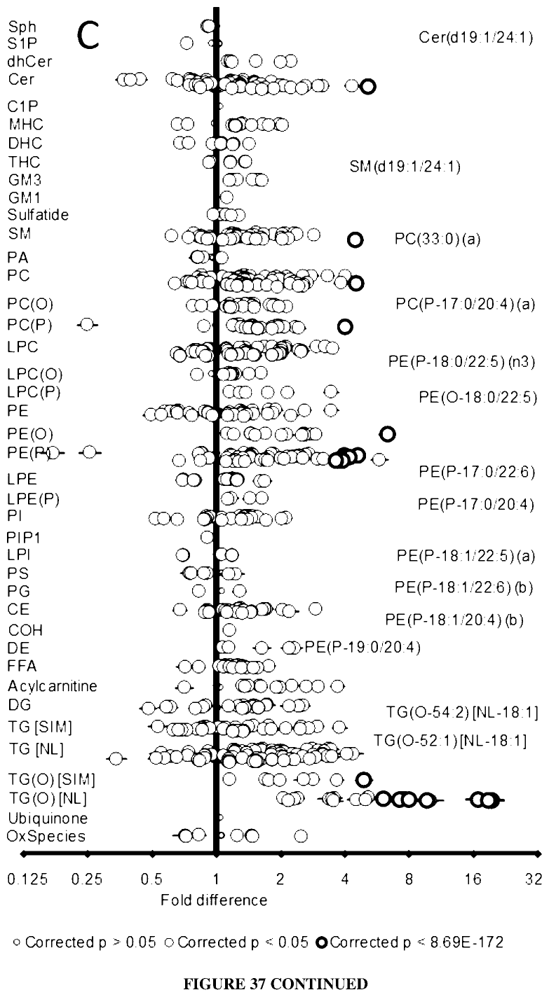

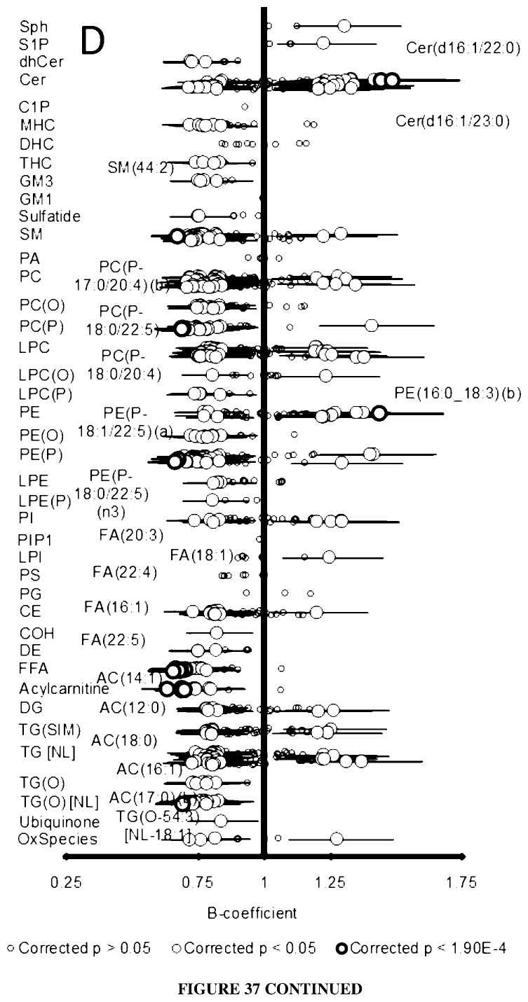

Associations of plasma lipids with growth trajectories. Associations were calculated using ordinal logistic regression. Growth trajectories, ordered as labelled in FIG. 37A were regressed against log transformed lipid concentrations, scaled to a unit variance, adjusting for sex. P-values were adjusted using the Benjamini-Hochberg procedure.

### Results

Growth trajectories and breastfeeding in BIS: Using WHO-standardised BMI measures at birth, 1, 6, 12 and 48 months old, we have calculated growth trajectories and identified three subgroups within the cohort: 1) A low-BMI group, exhibited by 20.7% of the cohort, with normal BMI at birth and a marked reduction in BMI over the first 12 months of life (relative to the cohort mean), increasing at 48 months but still below average; 2) an ‘average’ group containing 69.1% of the cohort, starting with a slightly elevated BMI which largely stays unchanged over 48 months; and 3) a rapid increase (adverse) group, exhibited by 10.3% of the cohort, starting with low BMI and rapidly increasing over 12 months, then stabilizing with an elevated BMI at 48 months (FIG. 37A). Comparison between groups of the proportion of breastfeeding children at 6 months showed all groups to be significantly different, with the proportion decreasing from groups 1 and 2 to the adverse growth group 3 (all pairwise differences p<0.05, FIG. 37B). Ordinal logistic regression analysis of duration of breastfeeding (<1 month, 1-6 month, 6-12 months >12 months) with growth trajectories showed a significant protective effect of breastfeeding against the adverse growth trajectory (odds ratio=0.705, p=3.6×10⁻⁶). This equates to a 65% risk reduction of being in the adverse growth trajectory group (obese) for those who were breast fed >12 months compared to <1 month.

Growth trajectories and plasma lipids: We used ordinal logistic regression to examine the association of plasma lipids at six months of age with the growth trajectories. We observed a strong association of alkyldiacylglycerol (TG(O) and ether phospholipids with growth trajectories. The direction of association suggested that these lipid species may be providing the protective effect against adverse growth trajectories leading to obesity in early childhood.

### Summary and Conclusions

Our results show a clear association between breast feeding and growth trajectories, between breast feeding and plasma lipids, and between plasma lipids and growth trajectories. In particular, the TG(O) species are prominent in both the latter two associations and support a protective role for breast milk alkyldiacylglycerols (TG(O)) against adverse growth trajectories leading to obesity.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety

Those of skill in the art will appreciate that, in light of the instant disclosure, various modifications and changes can be made in the particular embodiments exemplified without departing from the scope of the present disclosure. All such modifications and changes are intended to be included within the scope of the appended claims.

### Bibliography

Alshehry, Z. H., Barlow, C. K., Weir, J. M., Zhou, Y., McConville, M. J., and Meikle, P. J. (2015) An Efficient Single Phase Method for the Extraction of Plasma Lipids. Metabolites 5, 389-403
Alshehry Z H, Mundra P A, Barlow C K, Mellett N A, Wong G, McConville M J, et al. Plasma Lipidomic Profiles Improve on Traditional Risk Factors for the Prediction of Cardiovascular Events in Type 2 Diabetes Mellitus. Circulation. 2016;134(21):1637-50.
Caielli S, Banchereau J, and Pascual V. Neutrophils come of age in chronic inflammation. Curr Opin Immunol. 2012; 24(6):671-7.
Dogaru, C. M., Nyffenegger, D., Pescatore, A. M., Spycher, B. D., and Kuehni, C. E. (2014) Breastfeeding and childhood asthma: systematic review and meta-analysis. Am J Epidermol 179, 1153-1167.
Fahy E, Subramaniam S, Brown H A, Glass C K, Merrill A H, Murphy R C, Jr., Raetz, C R, Russell D W, Seyama Y, Shaw W, Shimizu T, Spener F, Van Meer G, Vannieuwenhza M S, White S H, Witztum J L, and Dennis E A. A comprehensive classification system for lipids. J. Lipid Res. 2005; 46:839-61.
Fahy E, Subramaniam S, Murphy R C, Nishijima M, Raetz C R, Shimizu T, Spener F, Van Meer G, Wakelam M J, and Dennis E A. Update of the LIPID MAPS comprehensive classification systems for lipids. J. Lipid Res. 2009; 50:S9-14.
Geserick, M., Vogel, M., Gausche, R., Lipek, T., Spielau, U., Keller, E., et al. (2018) Acceleration of BMI in Early Childhood and Risk of Sustained Obesity. New England Journal of Medicine 379, 1303-1312
Gray, L. E. K., Ponsonby, A. L., Collier, F., O'Hely, M., Sly, P. D., Ranganathan, S., et al. (2019) Deserters on the atopic march: Risk factors, immune profile, and clinical outcomes of food-sensitized-tolerant infants. Allergy.
Gray, L. E. K., Ponsonby, A. L., Lin, T. X., O'Hely, M., Collier, F., Ranganathan, S., et al. (2019) High incidence of respiratory disease in Australian infants despite low rate of maternal cigarette smoking. J Paediatr Child Health 55, 1437-1444.
Hidayat, K., Du, X., and Shi, B. M. (2018) Body fatness at a young age and risks of eight types of cancer: systematic review and meta-analysis of observational studies. Obes Rev 19, 1385-1394
Huynh K, Barlow C K, Jayawardana K S, Weir J M, Mellett N A, Cinel M, et al. High-throughput plasma lipidomics: Detailed mapping of the associations with cardiometabolic risk factors. Cell Chemical Biology. 2019;26(1):71-84.
Jang, J. E., Park, H. S., Yoo, H. J., Baek, I. J., Yoon, J. E., Ko, M. S., et al. (2017) Protective role of endogenous plasmalogens against hepatic steatosis and steatohepatitis in mice. Hepatology 66, 416-431
Juonala, M., Magnussen, C. G., Berenson, G. S., Venn, A., Burns, T. L., Sabin, M. A., et al. (2011) Childhood adiposity, adult adiposity, and cardiovascular risk factors. The New England journal of medicine 365, 1876-1885
Liebisch G, Vizcaino J A, Kofeler H, Trotzmuller M, Griffiths W J, Schmitz G, Spener F, and Wakelam M J. Shorthand notation for lipid structures derived from mass spectrometry. J. Lipid Res. 2013;54:1523-30.
Magnusson C D, Gudmundsdottir A V, and Haraldsson G G. Chemoenzymatic synthesis of a focused library of enantiopure structured 1-O-alkyl-2,3-diacyl-sn-glycerol type ether lipids. Tetrahedron. 2011;67:1821-36.
Mayer-Davis, E. J., Rifas-Shiman, S. L., Zhou, L., Hu, F. B., Colditz, G. A., and Gillman, M. W. (2006) Breast-feeding and risk for childhood obesity: does maternal diabetes or obesity status matter? Diabetes Care 29, 2231-2237.
Owen, C. G., Martin, R. M., Whincup, P. H., Smith, G. D., and Cook, D. G. (2005) Effect of infant feeding on the risk of obesity across the life course: a quantitative review of published evidence. Pediatrics 115, 1367-1377.
Paoletti R, Gotto A M, Jr., and Hajjar D P. Inflammation in atherosclerosis and implications for therapy. Circulation. 2004;109(23 Suppl 1):III20-6.
Paul, S., Lancaster, G. I., and Meikle, P. J. (2019) Plasmalogens: A potential therapeutic target for neurodegenerative and cardiometabolic disease. Prog Lipid Res 74, 186-195.
Rasmiena AA, Barlow C K, Stefanovic N, Huynh K, Tan R, Sharma A, et al. Plasmalogen modulation attenuates atherosclerosis in ApoE- and ApoE/GPx1-deficient mice. Atherosclerosis. 2015; 243(2):598-608.
Riordan, J., and Countryman, B. A. (1980) Part I: Infant Feeding Patterns Past and Present. JOGN Nursing 9, 207
Shi Y, Dayoub W, Chen G-R, and Lemaire M. Selective synthesis of 1-O-alkyl glycerol and diglycerol ethers by reductive alkylation of alcohols. Green Chemistry. 2010;12(12).
Uwaezuoke, S. N., Eneh, C. I., and Ndu, I. K. (2017) Relationship Between Exclusive Breastfeeding and Lower Risk of Childhood Obesity: A Narrative Review of Published Evidence. Clin Med Insights Pediatr 11, 1179556517690196.
Vuillermin, P., Saffery, R., Allen, K. J., Carlin, J. B., Tang, M. L., Ranganathan, S., et al. (2015) Cohort Profile: The Barwon Infant Study. International journal of epidemiology 44, 1148-1160.

Weir J M, Wong G, Barlow C K, Greeve M A, Kowalczyk A, Almasy L, et al. Plasma lipid profiling in a large population-based cohort. J Lipid Res. 2013;54(10):2898-908.

Yusuf S, Hawken S, Ounpuu S, Dans T, Avezum A, Lanas F, et al. Effect of potentially modifiable risk factors associated with myocardial infarction in 52 countries (the INTERHEART study): case-control study. Lancet. 2004;364(9438):937-52.

Yu, H., Dilbaz, S., CoBmann, J., Hoang, A. C., Diedrich, V., Herwig, A., et al. (2019) Breast milk alkylglycerols sustain beige adipocytes through adipose tissue macrophages. The Journal of Clinical Investigation 129, 2485-2499.

1. A composition comprising a mixture of ether lipid molecules of Formula (I): 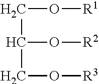
(I)
wherein
R¹ is an alkyl or alkenyl group;
R² is hydrogen or 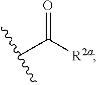
and
R³ is hydrogen, 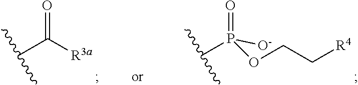
wherein
R^{2a} and R^{3a} are each an alkyl or alkenyl group;
R⁴ is —N(Me)₃⁺ or —NH₃⁺; and
wherein the composition is for in vivo maintenance of ether lipids at levels and/or ratios associated with a non-disease state, or wherein the composition is for in vivo modification of ether lipids towards levels and/or ratios associated with a non-disease state.

2. The composition according to claim 1, wherein the composition is for in vivo maintenance or in vivo modification of plasmanyl- and/or plasmenyl-phospholipid levels and/or ratios.

3. The composition according to claim 1 or claim 2, wherein the composition comprises ether lipid molecules having an 18:0 alkyl R¹ group, and ether lipid molecules having an 18:1 alkenyl R¹ group.

4. The composition according to any one of claims 1 to 3, wherein the composition is for in vivo maintenance of ether lipids at or in vivo modification of ether lipids towards an in vivo plasmalogen ether lipid profile in which the ether lipids have a molar ratio of 18:0 ether groups to 18:1 ether groups of from 1.2:1 to 2.5:1.

5. The composition according to any one of claims 1 to 4, wherein the composition is for in vivo maintenance of ether lipids at or in vivo modification of ether lipids towards an in vivo plasmalogen ether lipid profile in which the ether lipids have a molar percent of 18:0 ether groups in the range of from 32.6% to 45.8%, and a molar percent of 18:1 ether groups in the range of from 18.6% to 27.9%.

6. The composition according to any one of claims 1 to 5, wherein the composition comprises ether lipids having a molar ratio of 18:0 alkyl R¹ groups to 18:1 alkenyl R¹ groups in the range of from 1.2:1 to 2.5:1.

7. The composition according to any one of claims 1 to 6, wherein the composition comprises ether lipid molecules having an 18:1 alkenyl R¹ group, and ether lipid molecules having a 16:0 alkyl R¹ group.

8. The composition according to any one of claims 1 to 7, wherein the composition is for in vivo maintenance of ether lipids at or in vivo modification of ether lipids towards an in vivo plasmalogen ether lipid profile in which the ether lipids have a molar ratio of 18:1 ether groups to 16:0 ether groups in the range of from 0.5:1 to 1:1.

9. The composition according to any one of claims 1 to 8, wherein the composition is for in vivo maintenance of ether lipids at or in vivo modification of ether lipids towards an in vivo plasmalogen ether lipid profile in which the ether lipids have a molar percent of 18:lether groups in the range of from 18.6% to 27.9%, and a molar percent of 16:0 ether groups in the range of from 26.8% to 37.4%.

10. The composition according to any one of claims 1 to 9, wherein the composition comprises ether lipids having a molar ratio of 18:1 alkenyl R¹ groups to 16:0 alkyl R¹ groups in the range of from 0.5:1 to 1:1.

11. The composition according to any one of claims 1 to 10, wherein the composition comprises ether lipid molecules having an 18:0 alkyl R¹ group, and ether lipid molecules having a 16:0 alkyl R¹ group.

12. The composition according to any one of claims 1 to 11, wherein the composition is for in vivo maintenance of ether lipids at or in vivo modification of ether lipids towards an in vivo plasmalogen ether lipid profile in which the ether lipids have a molar ratio of 18:0 ether groups to 16:0 ether groups in the range of from 0.9:1 to 1.7:1.

13. The composition according to any one of claims 1 to 12, wherein the composition is for in vivo maintenance of ether lipids at or in vivo modification of ether lipids towards an in vivo plasmalogen ether lipid profile in which the ether lipids have a molar percent of 18:0 ether groups in the range of from 32.6% to 45.8%, and a molar percent of 16:0 ether groups in the range of from 26.8% to 37.4%.

14. The composition according to any one of claims 1 to 13, wherein the composition comprises ether lipids having a molar ratio of 18:0 alkyl R¹ groups to 16:0 alkyl R¹ groups in the range of from 0.9:1 to 1.7:1.

15. The composition according to any one of claims 1 to 14, wherein the composition comprises ether lipid molecules having an 18:1 alkenyl R¹ group, ether lipid molecules having a 18:0 alkyl R¹ group, and ether lipid molecules having a 16:0 alkyl R¹ group.

16. The composition according to any one of claims 1 to 15, wherein the composition is for in vivo maintenance of ether lipids at or in vivo modification of ether lipids towards an in vivo plasmalogen ether lipid profile in which the ether lipids have a molar ratio of 18:1 ether groups to 18:0 ether groups to 16:0 ether groups of about 1:1.7:1.4.

17. The composition according to any one of claims 1 to 16, wherein the composition is for in vivo maintenance of ether lipids at or in vivo modification of ether lipids towards an in vivo plasmalogen ether lipid profile in which the ether lipids have a molar percent of 18:1 ether groups in the range of from 18.6% to 27.9%, a molar percent of 18:0 ether groups in the range of from 32.6% to 45.8%, and a molar percent of 16:0 ether groups in the range of from 26.8% to 37.4%.

18. The composition according to any one of claims 1 to 17, wherein the composition comprises ether lipids having a molar ratio of 18:1 alkenyl R¹ groups to 18:0 alkyl R¹ groups to 16:0 alkyl R¹ groups of about 1:1.7:1.4.

19. The composition according to claim 19, wherein ether lipids having an 18:1 alkenyl R¹ group, ether lipids having an 18:0 alkyl R¹ group, and ether lipids having a 16:0 alkyl R¹ group together comprise at least 50% of the ether lipids in the composition.

20. The composition according to any one of claims 1 to 19, wherein the composition additionally comprises ether lipids having R¹ groups selected from the group consisting of 15:0 alkyl, 17:0 alkyl, 19:0 alkyl, 20:0 alkyl, and 20:1 alkenyl.

21. The composition according to any one of claims 1 to 20, wherein the composition comprises ether lipids wherein R² and R³ is hydrogen.

22. The composition according to any one of claims 1 to 20, wherein the composition comprises ether lipids in which R² is hydrogen and R³ is 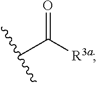
and
R^{3a} is selected from the group consisting of a saturated alkyl hydrocarbon, a monounsaturated alkenyl hydrocarbon and a polyunsaturated alkenyl hydrocarbon.

23. The composition according to any one of claims 1 to 22, wherein the composition comprises ether lipids in which R³ is hydrogen and R² is 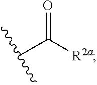
and
R^{2a} is selected from the group consisting of a saturated alkyl hydrocarbon, a monounsaturated alkenyl hydrocarbon and a polyunsaturated alkenyl hydrocarbon.

24. The composition according to any one of claims 1 to 23, wherein the composition comprises ether lipids in which
R² is: 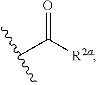
R³ is 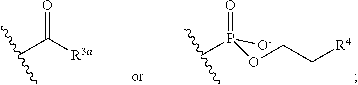
wherein
R^{2a} is selected from the group consisting of a saturated alkyl hydrocarbon, a monounsaturated alkenyl hydrocarbon and a polyunsaturated alkenyl hydrocarbon;
R^{3a} is selected from the group consisting of a saturated alkyl hydrocarbon, a monounsaturated alkenyl hydrocarbon and a polyunsaturated alkenyl hydrocarbon;
and
R⁴ is —N(Me)3⁺ or NH₃⁺.

25. The composition according to any one of claims 1 to 20 and 22 to 24, wherein the composition comprises ether lipid molecules having a 20:4 acyl alkenyl R² and/or R³ group, ether lipids having a 22:6 acyl alkenyl R² and/or R³ group, and ether lipids having an 18:2 acyl alkenyl R² and/or R³ group.

26. The composition according to any one of claims 1 to 20 and 22 to 25, wherein the composition is for in vivo maintenance of ether lipids at or in vivo modification of ether lipids towards an in vivo plasmalogen ether lipid profile in which the ether lipids have a molar ratio of 20:4 acyl alkenyl groups to 22:6 acyl alkenyl groups to 18:2 acyl alkenyl groups of about 3:1.2:1.

27. The composition according to any of claims 1 to 20 and 22 to 26, wherein the composition is for in vivo maintenance of ether lipids at or in vivo modification of ether lipids towards an in vivo plasmalogen ether lipid profile in which the ether lipids have acyl alkenyl groups in which the molar percent of 20:4 acyl alkenyl groups is in the range of from 31.3% to 52.5%, the molar percent of 22:6 acyl alkenyl groups is in the range of from 9.3% to 23.9%, and the molar percent of 18:2 acyl alkenyl groups is in the range of from 7.6% to 19.9%.

28. The composition according to any one of claims 1 to 20 and 22 to 27, wherein the composition comprises ether lipids having a molar ratio of 20:4 acyl alkenyl groups to 22:6 acyl alkenyl groups to 18:2 acyl alkenyl groups of about 3:1.2:1.

29. The composition according to any one of claims 1 to 28, wherein the composition comprises free fatty acids.

30. The composition according to any one of claims 1 to 29, wherein the composition comprises omega-3 or omega-6 fatty acids.

31. The composition according to any one of claims 1 to 30, wherein the composition is an ether lipid-containing composition according to the Examples.

32. A composition as claimed in any of claims 1 to 31, wherein the composition is in the form of a composition for addition to a food or beverage.

33. A composition as claimed in any of claims 1 to 31, wherein the composition is in the form of a product which is a dietary supplement, capsule, syrup, liquid, food or beverage.

34. A composition comprising a mixture of ether lipid molecules of Formula (I): 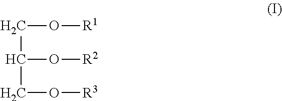
wherein
R¹ is an alkyl or alkenyl group;
R² is hydrogen or 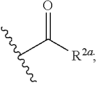
and
R³ is hydrogen, 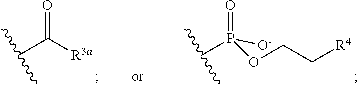
wherein
R^{2a} and R^{3a} are each an alkyl or alkenyl group; and
R⁴ is —N(Me)₃⁺ or —NH₃⁺, and
wherein the composition is present in the form of a product which is a liquid infant formula milk, an infant formula milk powder, a supplement for addition to infant formula milk, a supplement for addition to infant food, or an infant dietary supplement.

35. The composition according to claim 34, wherein the composition comprises ether lipid molecules having an 18:0 R¹ group, and ether lipid molecules having an 18:1 R¹ group.

36. The composition according to claim 34 or claim 35, wherein the composition is for in vivo maintenance of ether lipids at or in vivo modification of ether lipids towards an in vivo plasmalogen ether lipid profile in which the ether lipids have a molar ratio of 18:0 ether groups to 18:1 ether groups of from 0.74:1 to 1.60:1.

37. The composition according to any of claims 34 to 36, wherein the composition is for in vivo maintenance of ether lipids at or in vivo modification of ether lipids towards an in vivo plasmalogen ether lipid profile in which the ether lipids have a molar percent of 18:0 ether groups in the range of from 27.7% to 39.6%, and a molar percent of 18:1 ether groups in the range of from 24.7% to 37.4%.

38. The composition according to any one of claims 34 to 37, wherein the composition comprises ether lipids having a molar ratio of 18:0 R¹ groups to 18:1 R¹ groups in the range of from 0.30:1 to 1.20:1.

39. The composition according to any one of claims 34 to 38, wherein the composition comprises ether lipid molecules having an 18:1 R¹ group, and ether lipid molecules having a 16:0 R¹ group.

40. The composition according to any one of claims 34 to 39, wherein the composition is for in vivo maintenance of ether lipids at or in vivo modification of ether lipids towards an in vivo plasmalogen ether lipid profile in which the ether lipids have a molar ratio of 16:0 ether groups to 18:1 ether groups in the range of from 1.24:1 to 0.59:1.

41. The composition according to any one of claims 34 to 40, wherein the composition is for in vivo maintenance of ether lipids at or in vivo modification of ether lipids towards an in vivo plasmalogen ether lipid profile in which the ether lipids have a molar percent of 18:1 ether groups in the range of from 24.7% to 37.4%, and a molar percent of 16:0 ether groups in the range of from 30.1% to 41.7%.

42. The composition according to any one of claims 34 to 41, wherein the composition comprises ether lipids having a molar ratio of 18:1 R¹ groups to 16:0 R¹ groups in the range of from 1:0.55 to 1:2.3.

43. The composition according to any one of claims 34 to 42, wherein the composition comprises ether lipid molecules having an 18:0 R¹ group, and ether lipid molecules having a 16:0 R¹ group.

44. The composition according to any one of claims 34 to 43, wherein the composition is for in vivo maintenance of ether lipids at or in vivo modification of ether lipids towards an in vivo plasmalogen ether lipid profile in which the ether lipids have a molar ratio of 18:0 ether groups to 16:0 ether groups in the range of from 0.66:1 to 1.3:1.

45. The composition according to any one of claims 34 to 44, wherein the composition is for in vivo maintenance of ether lipids at or in vivo modification of ether lipids towards an in vivo plasmalogen ether lipid profile in which the ether lipids have a molar percent of 18:0 ether groups in the range of from 27.7% to 39.6%, and a molar percent of 16:0 ether groups in the range of from 30.1% to 41.7%.

46. The composition according to any one of claims 34 to 45, wherein the composition comprises ether lipids having a molar ratio of 18:0 R¹ groups to 16:0 R¹ groups in the range of from 0.44:1 to 1.82:1.

47. The composition according to any one of claims 34 to 46, wherein the composition comprises ether lipid molecules having an 18:1 R¹ group, ether lipid molecules having a 18:0 R¹ group, and ether lipid molecules having a 16:0 R¹ group.

48. The composition according to any one of claims 34 to 47, wherein the composition is for in vivo maintenance of ether lipids at or in vivo modification of ether lipids towards an in vivo plasmalogen ether lipid profile in which the ether lipids have a molar ratio of 18:1 ether groups to 18:0 ether groups to 16:0 alkyl ether groups of about 0.9:1.0:1.05.

49. The composition according to any one of claims 34 to 48, wherein the composition is for in vivo maintenance of ether lipids at or in vivo modification of ether lipids towards an in vivo plasmalogen ether lipid profile in which the ether lipids have a molar percent of 18:1 ether groups in the range of from 24.7% to 37.4%, a molar percent of 18:0 ether groups in the range of from 27.7% to 39.6%, and a molar percent of 16:0 ether groups in the range of from 30.1% to 41.7%.

50. The composition according to any one of claims 34 to 49, wherein the composition comprises ether lipids having a molar ratio of 18:0 R¹ groups to 16:0 R¹ groups to 18:1 R¹ groups is in the range of from 0.5:1:3 to 2:1:1.

51. The composition according to any of claims 34 to 50, wherein ether lipids having an 18:1 R¹ group, ether lipids having an 18:0 R¹ group, and ether lipids having a 16:0 R¹ group together comprise at least 50% of the ether lipids in the composition.

52. The composition according to any one of claims 34 to 51, wherein the composition additionally comprises ether lipids having R¹ groups selected from the group consisting of 16:0, 18:2, 20:0 and 20:1.

53. The composition according to any one of claims 34 to 52, wherein the composition comprises ether lipids wherein R² and R³ is hydrogen.

54. The composition according to any one of claims 34 to 53, wherein the composition comprises ether lipids in which R² is hydrogen and R³ is 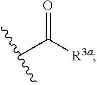
and
R^{3a} is selected from the group consisting of a saturated alkyl hydrocarbon, a monounsaturated alkenyl hydrocarbon and a polyunsaturated alkenyl hydrocarbon.

55. The composition according to any one of claims 34 to 54, wherein the composition comprises ether lipids in which R³ is hydrogen and R² is 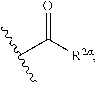
and
R^{2a} is selected from the group consisting of a saturated alkyl hydrocarbon, a monounsaturated alkenyl hydrocarbon and a polyunsaturated alkenyl hydrocarbon.

56. The composition according to any one of claims 34 to 55, wherein the composition comprises ether lipids in which
R² is: 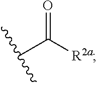
R³ is 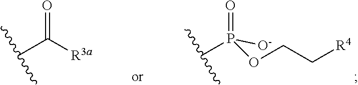
wherein
R^{2a} is selected from the group consisting of a saturated alkyl hydrocarbon, a monounsaturated alkenyl hydrocarbon and a polyunsaturated alkenyl hydrocarbon;
R^{3a} is selected from the group consisting of a saturated alkyl hydrocarbon, a monounsaturated alkenyl hydrocarbon and a polyunsaturated alkenyl hydrocarbon; and
R⁴ is —N(Me)₃⁺ or NH₃⁺.

57. The composition according to any one of claims 34 to 56, wherein the composition comprises free fatty acids.

58. The composition according to any one of claims 34 to 57, wherein the composition comprises omega-3 or omega-6 fatty acids.

59. A composition as claimed in any of claims 34 to 58, wherein the composition comprises ether lipid molecules of Formula (I) in an amount such that, when present in liquid infant formula milk, the concentration of total ether lipid molecules of Formula (I) is in the range of from 75 to 140 μM.

60. The composition according to any one of claims 1 to 59, wherein the composition is prepared by mixing a plurality of ether lipids, in ratios and/or levels corresponding with ratios and/or levels associated with a non-disease state in vivo.

61. A method of maintaining ether lipids in a subject at levels and/or ratios associated with a non-disease state, or of modifying ether lipids in a subject towards levels and/or ratios associated with a non-disease state, comprising administering an effective amount of a composition of any of claims 1 to 60 to the subject.

62. A method as claimed in claim 61, wherein the method is for maintenance or modification of plasmanyl- and/or plasmenyl-phospholipid levels and/or ratios in a subject.

63. A method of assessing a subject for or with a metabolic disease, diabetes, cardiovascular disease, obesity, overweight, fatty liver disease, an inflammatory condition or dyslipidemia in a tissue or a risk of developing same, the method comprising measuring the relative abundance of one or more ether lipid side chains in a biological sample from a subject to obtain a subject ether lipid side chain profile, and (ii) determining the similarity or difference between the ether lipid side chain profile obtained in (i) and a reference ether lipid side chain profile.

64. A method of treating or preventing metabolic disease, diabetes, cardiovascular disease, obesity, overweight, fatty liver disease, an inflammatory condition or dyslipidemia in a subject, the method comprising (i) determining the relative abundance of one or more ether lipid side chains in a biological sample from a subject to obtain a subject ether lipid side chain profile, and (ii) administering a composition of any one of claims 1 to 60 contingent upon the similarity or difference between the ether lipid side chain profile obtained in (i) and a reference ether lipid side chain profile.

65. The method of claim 63 or 64, wherein the reference ether lipid side chain profile is the profile characteristic of a healthy individual and comprises:
ether lipids having a molar ratio of 18:1 alkenyl ether to 18:0 alkyl ether to 16:0 alkyl ether groups of about 1:1.7:1.4;
and/or
ether lipids having a molar percent of 18:1 alkenyl ether groups in the range of from 18.6% to 27.9%, a molar percent of 18:0 alkyl ether groups in the range of from 32.6% to 45.8%, and a molar percent of 16:0 alkyl ether groups in the range of from 26.8% to 37.4%.

66. A method of treating or preventing metabolic disease, diabetes, cardiovascular disease, obesity, overweight, fatty liver disease, an inflammatory condition or dyslipidemia in a subject, the method comprising administering an effective amount of a composition of any one of claims 1 to 60 to the subject.

67. A method of preventing asthma, an inflammatory condition, obesity or overweight in an infant subject, the method comprising administering an effective amount of a composition of any of claims 1 to 60 to the infant subject.

68. A composition of any of claims 1 to 60 for use in therapy.

69. A composition of any of claims 1 to 60 for use in treating or preventing metabolic disease, diabetes, cardiovascular disease, obesity, overweight, fatty liver disease, an inflammatory condition or dyslipidemia in a subject.

70. A composition of any of claims 1 to 60 for use in preventing asthma, inflammatory condition, obesity or overweight in an infant subject.

71. Use of a composition of any of claims 1 to 60 for the manufacture of a medicament for the treatment or prevention of metabolic disease, diabetes, cardiovascular disease, obesity, overweight, fatty liver disease, an inflammatory condition or dyslipidemia in a subject.

72. Use of a composition of any of claims 1 to 60 for the manufacture of a medicament for the prevention of asthma, an inflammatory condition, obesity or overweight in an infant subject.
